(19) United States
(12) Patent Application Publication
(10) Pub. No.: US 2014/0024076 A1
(43) Pub. Date: Jan. 23, 2014

(54) Expression Of Soluble Viral Fusion Glycoproteins In Mammalian Cells

(71) Applicants: Roderick Tang, San Mateo, CA (US); Gregory M. Hayes, San Francisco, CA (US); Heather Lawlor, Gaithersburg, MD (US); Peifeng Chen, Boyds, MD (US); Yi Liu, Cupertino, CA (US)

(72) Inventors: Roderick Tang, San Mateo, CA (US); Gregory M. Hayes, San Francisco, CA (US); Heather Lawlor, Gaithersburg, MD (US); Peifeng Chen, Boyds, MD (US); Yi Liu, Cupertino, CA (US)

(73) Assignee: MEDIMMUNE, LLC., Gaithersburg, MD (US)

(21) Appl. No.: 13/980,712

(22) PCT Filed: Jan. 27, 2012
(86) PCT No.: PCT/US2012/022997

§ 371 Date: Oct. 4, 2013

Related U.S. Application Data
(60) Provisional application No. 61/437,531, filed on Jan. 28, 2011.

(51) Int. Cl. C07K 14/005

(52) U.S. Cl. CPC C07K 14/005
USPC 435/69.1; 536/23.72; 435/320.1; 435/358

(57) ABSTRACT

The technology relates in part to production (i.e., expression) of recombinant viral fusion glycoproteins and nucleic acids that encode such viral fusion glycoproteins. In some embodiments, human respiratory syncytial virus fusion protein (RSV-F) and human parainfluenza virus 3 fusion protein (hPIV3-F) are expressed.

### CLAIM OF PRIORITY

This application claims the benefit of prior U.S. Provisional Application No. 61/437,531, filed on Jan. 28, 2011, which is incorporated by reference in its entirety.

### FIELD

The technology relates in part to production (i.e., expression) of recombinant viral fusion glycoproteins and nucleic acids that encode such viral fusion glycoproteins. In some embodiments, methods for producing recombinant human respiratory syncytial virus fusion protein (RSV-F) and human parainfluenza virus 3 fusion protein (hPIV3-F), and nucleic acids encoding such proteins, are provided.

### BACKGROUND

During viral infection, viral fusion glycoproteins mediate entry of a virus into a host cell. A viral fusion protein projects from the viral envelope surface as a trimer, and mediates cell entry by inducing fusion between the viral envelope and the cell membrane. There are two classes of viral fusion proteins: type I and type II. Type I viral fusion proteins include, but are not limited to, fusion proteins of paramyxoviruses, retroviruses, coronaviruses, orthomyxoviruses, and filoviruses. Type II viral fusion proteins include, but are not limited to, the fusion proteins of alphaviruses and flaviviruses. Both type I and type II viral fusion proteins are arranged as trimers at fusion. Type I viral fusion proteins are synthesized as monomers and trimerize after cotranslational insertion into the membrane of the endoplasmic reticulum, glycosylation, and folding. Following trimerization, type I viral fusion precursor (F₀) proteins are cleaved and activated by host proteases. In paramyxoviruses, for example, activated type I viral fusion proteins are composed of a membrane-anchored subunit and a membrane-distal subunit, which are named F1 and F2, respectively. The membrane-anchored subunit contains a transmembrane domain and a new hydrophobic amino terminus, known as the fusion peptide.

Paramyxoviral fusion proteins can include fusion proteins from viruses such as, for example, respiratory syncytial virus (RSV) and human parainfluenza viruses (hPIV). RSV infection gives rise to serious lower respiratory tract illness, particularly in premature infants. Prophylaxis with palivizumab, a monoclonal antibody that neutralizes RSV, can prevent lower respiratory tract infection caused by RSV in premature infants, however there are no vaccines approved for RSV. hPIV also is a common cause of lower respiratory tract infection in young children. There are four serotypes of hPIV which include hPIV-1 (most common cause of croup and other upper and lower respiratory tract illnesses); hPIV-2 (causes croup and other upper and lower respiratory tract illnesses); hPIV-3 (associated with bronchiolitis and pneumonia); and hPIV-4. Like RSV, there are no vaccines approved for hPIV.

### SUMMARY

Provided in some embodiments is an isolated nucleic acid comprising a nucleotide sequence having a GC content of about 51% or greater that encodes a soluble viral fusion protein comprising an amino acid sequence 90% or more identical to SEQ ID NO: 7.

In some embodiments, the nucleotide sequence encodes a protein comprising an amino acid sequence 91% or more identical to SEQ ID NO: 7. In some embodiments, the nucleotide sequence encodes a protein comprising an amino acid sequence 92% or more identical to SEQ ID NO: 7. In some embodiments, the nucleotide sequence encodes a protein comprising an amino acid sequence 93% or more identical to SEQ ID NO: 7. In some embodiments, the nucleotide sequence encodes a protein comprising an amino acid sequence 94% or more identical to SEQ ID NO: 7. In some embodiments, the nucleotide sequence encodes a protein comprising an amino acid sequence 95% or more identical to SEQ ID NO: 7. In some embodiments, the nucleotide sequence encodes a protein comprising an amino acid sequence 96% or more identical to SEQ ID NO: 7. In some embodiments, the nucleotide sequence encodes a protein comprising an amino acid sequence 97% or more identical to SEQ ID NO: 7. In some embodiments, the nucleotide sequence encodes a protein comprising an amino acid sequence 98% or more identical to SEQ ID NO: 7. In some embodiments, the nucleotide sequence encodes a protein comprising an amino acid sequence 99% or more identical to SEQ ID NO: 7. In some embodiments, the nucleotide sequence encodes a protein comprising an amino acid sequence of SEQ ID NO: 7.

In some embodiments, the soluble viral fusion protein lacks a functional membrane association region. At times, the soluble viral fusion protein lacks the C-terminal transmembrane region amino acids corresponding to amino acids 525 to 574 of SEQ ID NO: 2.

In some embodiments, the GC content of the nucleotide sequence is about 45% or greater. In some embodiments, the GC content of the nucleotide sequence is about 46% or greater. In some embodiments, the GC content of the nucleotide sequence is about 47% or greater. In some embodiments, the GC content of the nucleotide sequence is about 48% or greater. In some embodiments, the GC content of the nucleotide sequence is about 49% or greater. In some embodiments, the GC content of the nucleotide sequence is about 50% or greater. In some embodiments, the GC content of the nucleotide sequence is about 51% or greater. In some embodiments, the GC content of the nucleotide sequence is about 52% or greater. In some embodiments, the GC content of the nucleotide sequence is about 53% or greater. In some embodiments, the GC content of the nucleotide sequence is about 54% or greater. In some embodiments, the GC content of the nucleotide sequence is about 55% or greater. In some embodiments, the GC content of the nucleotide sequence is about 56% or greater. In some embodiments, the GC content of the nucleotide sequence is about 57% or greater. In some embodiments, the GC content of the nucleotide sequence is about 58% or greater. In some embodiments, the GC content of the nucleotide sequence is about 59% or greater. In some embodiments, the GC content of the nucleotide sequence is about 60% or greater. In some embodiments, the GC content of the nucleotide sequence is about 61% or greater. In some embodiments, the GC content of the nucleotide sequence is about 62% or greater. In some embodiments, the GC content of the nucleotide sequence is about 63% or greater. In some embodiments, the GC content of the nucleotide sequence is about 64% or greater. In some embodiments, the GC content of the nucleotide sequence is about 65% or greater. In some embodiments, the GC content of the nucleotide sequence is about 70% or greater. In some embodiments, the GC content of the nucleotide sequence is about 75% or greater. In some embodiments, the GC content is about 80% or greater. In some embodiments, the GC content is about 85% or greater. In some embodiments, the GC content is about 90% or greater. In some embodiments, the GC content is about 95% or greater. In some embodiments, the GC content is about 99% or greater.

In some embodiments, the GC3 content of the nucleotide sequence is about 70% or greater. In some embodiments, the GC3 content of the nucleotide sequence is about 75% or greater. In some embodiments, the GC3 content of the nucleotide sequence is about 76% or greater. In some embodiments, the GC3 content of the nucleotide sequence is about 77% or greater. In some embodiments, the GC3 content of the nucleotide sequence is about 78% or greater. In some embodiments, the GC3 content of the nucleotide sequence is about 79% or greater. In some embodiments, the GC3 content of the nucleotide sequence is about 80% or greater. In some embodiments, the GC3 content of the nucleotide sequence is about 85% or greater. In some embodiments, the GC3 content of the nucleotide sequence is about 90% or greater. In some embodiments, the GC3 content of the nucleotide sequence is about 95% or greater. In some embodiments, the GC3 content of the nucleotide sequence is about 96% or greater. In some embodiments, the GC3 content of the nucleotide sequence is about 97% or greater. In some embodiments, the GC3 content of the nucleotide sequence is about 98% or greater. In some embodiments, the GC3 content of the nucleotide sequence is about 99% or greater. In some embodiments, the GC3 content of the nucleotide sequence is about 100%. Certain embodiments are directed to a nucleic acid comprising the nucleotide sequence of SEQ ID NO: 6. Certain embodiments are directed to a nucleic acid comprising the nucleotide sequence of SEQ ID NO: 5.

In some embodiments, the nucleotide sequence is 60% or more identical to SEQ ID NO: 3. In some embodiments, the nucleotide sequence is 61% or more identical to SEQ ID NO: 3. In some embodiments, the nucleotide sequence is 62% or more identical to SEQ ID NO: 3. In some embodiments, the nucleotide sequence is 63% or more identical to SEQ ID NO: 3. In some embodiments, the nucleotide sequence is 64% or more identical to SEQ ID NO: 3. In some embodiments, the nucleotide sequence is 65% or more identical to SEQ ID NO: 3. In some embodiments, the nucleotide sequence is 66% or more identical to SEQ ID NO: 3. In some embodiments, the nucleotide sequence is 67% or more identical to SEQ ID NO: 3. In some embodiments, the nucleotide sequence is 68% or more identical to SEQ ID NO: 3. In some embodiments, the nucleotide sequence is 69% or more identical to SEQ ID NO: 3. In some embodiments, the nucleotide sequence is 70% or more identical to SEQ ID NO: 3. In some embodiments, the nucleotide sequence is 71% or more identical to SEQ ID NO: 3. In some embodiments, the nucleotide sequence is 72% or more identical to SEQ ID NO: 3. In some embodiments, the nucleotide sequence is 73% or more identical to SEQ ID NO: 3. In some embodiments, the nucleotide sequence is 74% or more identical to SEQ ID NO: 3. In some embodiments, the nucleotide sequence is 75% or more identical to SEQ ID NO: 3. In some embodiments, the nucleotide sequence is 76% or more identical to SEQ ID NO: 3. In some embodiments, the nucleotide sequence is 77% or more identical to SEQ ID NO: 3. In some embodiments, the nucleotide sequence is 78% or more identical to SEQ ID NO: 3. In some embodiments, the nucleotide sequence is 79% or more identical to SEQ ID NO: 3. In some embodiments, the nucleotide sequence is 80% or more identical to SEQ ID NO: 3. In some embodiments, the nucleotide sequence is 85% or more identical to SEQ ID NO: 3. In some embodiments, the nucleotide sequence is 90% or more identical to SEQ ID NO: 3. In some embodiments, the nucleotide sequence is 95% or more identical to SEQ ID NO: 3. In some embodiments, the nucleotide sequence is 99% or more identical to SEQ ID NO: 3.

In some embodiments, the nucleic acid further comprises a cis-regulatory element in functional association with the nucleotide sequence. Sometimes the cis-regulatory element comprises a post transcriptional processing element. At times, the post transcriptional regulatory element is from woodchuck hepatitis virus. In some embodiments the nucleic acid is in an expression vector. In some cases, the nucleic acid encodes a protein comprising a tag.

In some embodiments, the nucleic acid is codon optimized and comprises a nucleotide sequence that (i) has a GC content of about 46% or greater, and (ii) encodes a soluble viral fusion protein that is about 90% or more identical to SEQ ID NO: 7.

In some embodiments, the nucleic acid has the nucleotide sequence of SEQ ID NO: 4.

Also provided in certain embodiments is an isolated nucleic acid comprising a nucleotide sequence (i) having a GC content of about 51% or greater, (ii) that is 73% or more identical to SEQ ID NO: 1, and (iii) that encodes a viral fusion protein comprising an amino acid sequence 90% or more identical to SEQ ID NO: 2.

In some embodiments, the nucleotide sequence encodes a protein comprising an amino acid sequence 91% or more identical to SEQ ID NO: 2. In some embodiments, the nucleotide sequence encodes a protein comprising an amino acid sequence 92% or more identical to SEQ ID NO: 2. In some embodiments, the nucleotide sequence encodes a protein comprising an amino acid sequence 93% or more identical to SEQ ID NO: 2. In some embodiments, the nucleotide sequence encodes a protein comprising an amino acid sequence 94% or more identical to SEQ ID NO: 2. In some embodiments, the nucleotide sequence encodes a protein comprising an amino acid sequence 95% or more identical to SEQ ID NO: 2. In some embodiments, the nucleotide sequence encodes a protein comprising an amino acid sequence 96% or more identical to SEQ ID NO: 2. In some embodiments, the nucleotide sequence encodes a protein comprising an amino acid sequence 97% or more identical to SEQ ID NO: 2. In some embodiments, the nucleotide sequence encodes a protein comprising an amino acid sequence 98% or more identical to SEQ ID NO: 2. In some embodiments, the nucleotide sequence encodes a protein comprising an amino acid sequence 99% or more identical to SEQ ID NO: 2. In some embodiments, the nucleotide sequence encodes a protein comprising an amino acid sequence of SEQ ID NO: 2.

In some embodiments, the GC content of the nucleotide sequence is about 45% or greater. In some embodiments, the GC content of the nucleotide sequence is about 46% or greater. In some embodiments, the GC content of the nucleotide sequence is about 47% or greater. In some embodiments, the GC content of the nucleotide sequence is about 48% or greater. In some embodiments, the GC content of the nucleotide sequence is about 49% or greater. In some embodiments, the GC content of the nucleotide sequence is about 50% or greater. In some embodiments, the GC content of the nucleotide sequence is about 51% or greater. In some embodiments, the GC content of the nucleotide sequence is about 52% or greater. In some embodiments, the GC content of the nucleotide sequence is about 53% or greater. In some embodiments, the GC content of the nucleotide sequence is about 54% or greater. In some embodiments, the GC content of the nucleotide sequence is about 55% or greater. In some embodiments, the GC content of the nucleotide sequence is about 56% or greater. In some embodiments, the GC content of the nucleotide sequence is about 57% or greater. In some embodiments, the GC content of the nucleotide sequence is about 58% or greater. In some embodiments, the GC content of the nucleotide sequence is about 59% or greater. In some embodiments, the GC content of the nucleotide sequence is about 60% or greater. In some embodiments, the GC content of the nucleotide sequence is about 61% or greater. In some embodiments, the GC content of the nucleotide sequence is about 62% or greater. In some embodiments, the GC content of the nucleotide sequence is about 63% or greater. In some embodiments, the GC content of the nucleotide sequence is about 64% or greater. In some embodiments, the GC content of the nucleotide sequence is about 65% or greater. In some embodiments, the GC content of the nucleotide sequence is about 70% or greater. In some embodiments, the GC content of the nucleotide sequence is about 75% or greater. In some embodiments, the GC content is about 80% or greater. In some embodiments, the GC content is about 85% or greater. In some embodiments, the GC content is about 90% or greater. In some embodiments, the GC content is about 95% or greater. In some embodiments, the GC content is about 99% or greater.

In some embodiments, the GC3 content of the nucleotide sequence is about 70% or greater. In some embodiments, the GC3 content of the nucleotide sequence is about 75% or greater. In some embodiments, the GC3 content of the nucleotide sequence is about 76% or greater. In some embodiments, the GC3 content of the nucleotide sequence is about 77% or greater. In some embodiments, the GC3 content of the nucleotide sequence is about 78% or greater. In some embodiments, the GC3 content of the nucleotide sequence is about 79% or greater. In some embodiments, the GC3 content of the nucleotide sequence is about 80% or greater. In some embodiments, the GC3 content of the nucleotide sequence is about 85% or greater. In some embodiments, the GC3 content of the nucleotide sequence is about 90% or greater. In some embodiments, the GC3 content of the nucleotide sequence is about 95% or greater. In some embodiments, the GC3 content of the nucleotide sequence is about 96% or greater. In some embodiments, the GC3 content of the nucleotide sequence is about 97% or greater. In some embodiments, the GC3 content of the nucleotide sequence is about 98% or greater. In some embodiments, the GC3 content of the nucleotide sequence is about 99% or greater. In some embodiments, the GC3 content of the nucleotide sequence is about 100%. Certain embodiments are directed to a nucleic acid comprising the nucleotide sequence of SEQ ID NO: 17.

In some embodiments, the nucleotide sequence is 60% or more identical to SEQ ID NO: 1. In some embodiments, the nucleotide sequence is 61% or more identical to SEQ ID NO: 1. In some embodiments, the nucleotide sequence is 62% or more identical to SEQ ID NO: 1. embodiments, the nucleotide sequence is 63% or more identical to SEQ ID NO: 1. In some embodiments, the nucleotide sequence is 64% or more identical to SEQ ID NO: 1. In some embodiments, the nucleotide sequence is 65% or more identical to SEQ ID NO: 1. In some embodiments, the nucleotide sequence is 66% or more identical to SEQ ID NO: 1. In some embodiments, the nucleotide sequence is 67% or more identical to SEQ ID NO: 1. In some embodiments, the nucleotide sequence is 68% or more identical to SEQ ID NO: 1. In some embodiments, the nucleotide sequence is 69% or more identical to SEQ ID NO: 1. In some embodiments, the nucleotide sequence is 70% or more identical to SEQ ID NO: 1. In some embodiments, the nucleotide sequence is 71% or more identical to SEQ ID NO: 1. In some embodiments, the nucleotide sequence is 72% or more identical to SEQ ID NO: 1. In some embodiments, the nucleotide sequence is 73% or more identical to SEQ ID NO: 1. In some embodiments, the nucleotide sequence is 74% or more identical to SEQ ID NO: 1. In some embodiments, the nucleotide sequence is 75% or more identical to SEQ ID NO: 1. In some embodiments, the nucleotide sequence is 76% or more identical to SEQ ID NO: 1. In some embodiments, the nucleotide sequence is 77% or more identical to SEQ ID NO: 1. In some embodiments, the nucleotide sequence is 78% or more identical to SEQ ID NO: 1. In some embodiments, the nucleotide sequence is 79% or more identical to SEQ ID NO: 1. In some embodiments, the nucleotide sequence is 80% or more identical to SEQ ID NO: 1. In some embodiments, the nucleotide sequence is 85% or more identical to SEQ ID NO: 1. In some embodiments, the nucleotide sequence is 90% or more identical to SEQ ID NO: 1. In some embodiments, the nucleotide sequence is 95% or more identical to SEQ ID NO: 1. In some embodiments, the nucleotide sequence is 99% or more identical to SEQ ID NO: 1.

In some embodiments, the nucleic acid further comprises a cis-regulatory element in functional association with the nucleotide sequence. Sometimes the cis-regulatory element comprises a post transcriptional processing element. At times, the post transcriptional regulatory element is from woodchuck hepatitis virus. In some embodiments the nucleic acid is in an expression vector. In some cases, the nucleic acid encodes a protein comprising a tag.

Also provided is an isolated nucleic acid comprising a nucleotide sequence having a GC content of about 51% or greater that encodes a viral fusion protein comprising an amino acid sequence 90% or more identical to SEQ ID NO: 12.

In some embodiments, the nucleotide sequence encodes a protein comprising an amino acid sequence 91% or more identical to SEQ ID NO: 12. In some embodiments, the nucleotide sequence encodes a protein comprising an amino acid sequence 92% or more identical to SEQ ID NO: 12. In some embodiments, the nucleotide sequence encodes a protein comprising an amino acid sequence 93% or more identical to SEQ ID NO: 12. In some embodiments, the nucleotide sequence encodes a protein comprising an amino acid sequence 94% or more identical to SEQ ID NO: 12. In some embodiments, the nucleotide sequence encodes a protein comprising an amino acid sequence 95% or more identical to SEQ ID NO: 12. In some embodiments, the nucleotide sequence encodes a protein comprising an amino acid sequence 96% or more identical to SEQ ID NO: 12. In some embodiments, the nucleotide sequence encodes a protein comprising an amino acid sequence 97% or more identical to SEQ ID NO: 12. In some embodiments, the nucleotide sequence encodes a protein comprising an amino acid sequence 98% or more identical to SEQ ID NO: 12. In some embodiments, the nucleotide sequence encodes a protein comprising an amino acid sequence 99% or more identical to SEQ ID NO: 12. In some embodiments, the nucleotide sequence encodes a protein comprising an amino acid sequence of SEQ ID NO: 12.

In some embodiments, the soluble viral fusion protein lacks a functional membrane association region. At times, the soluble viral fusion protein lacks the C-terminal transmembrane region amino acids corresponding to amino acids 489 to 539 of SEQ ID NO: 9.

In some embodiments, the GC content of the nucleotide sequence is about 45% or greater. In some embodiments, the GC content of the nucleotide sequence is about 46% or greater. In some embodiments, the GC content of the nucleotide sequence is about 47% or greater. In some embodiments, the GC content of the nucleotide sequence is about 48% or greater. In some embodiments, the GC content of the nucleotide sequence is about 49% or greater. In some embodiments, the GC content of the nucleotide sequence is about 50% or greater. In some embodiments, the GC content of the nucleotide sequence is about 51% or greater. In some embodiments, the GC content of the nucleotide sequence is about 52% or greater. In some embodiments, the GC content of the nucleotide sequence is about 53% or greater. In some embodiments, the GC content of the nucleotide sequence is about 54% or greater. In some embodiments, the GC content of the nucleotide sequence is about 55% or greater. In some embodiments, the GC content of the nucleotide sequence is about 56% or greater. In some embodiments, the GC content of the nucleotide sequence is about 57% or greater. In some embodiments, the GC content of the nucleotide sequence is about 58% or greater. In some embodiments, the GC content of the nucleotide sequence is about 59% or greater. In some embodiments, the GC content of the nucleotide sequence is about 60% or greater. In some embodiments, the GC content of the nucleotide sequence is about 65% or greater. In some embodiments, the GC content of the nucleotide sequence is about 70% or greater. In some embodiments, the GC content of the nucleotide sequence is about 75% or greater. In some embodiments, the GC content is about 80% or greater. In some embodiments, the GC content is about 85% or greater. In some embodiments, the GC content is about 90% or greater. In some embodiments, the GC content is about 95% or greater. In some embodiments, the GC content is about 99% or greater.

In some embodiments, the GC3 content of the nucleotide sequence is about 70% or greater. In some embodiments, the GC3 content of the nucleotide sequence is about 75% or greater. In some embodiments, the GC3 content of the nucleotide sequence is about 76% or greater. In some embodiments, the GC3 content of the nucleotide sequence is about 77% or greater. In some embodiments, the GC3 content of the nucleotide sequence is about 78% or greater. In some embodiments, the GC3 content of the nucleotide sequence is about 79% or greater. In some embodiments, the GC3 content of the nucleotide sequence is about 80% or greater. In some embodiments, the GC3 content of the nucleotide sequence is about 85% or greater. In some embodiments, the GC3 content of the nucleotide sequence is about 90% or greater. In some embodiments, the GC3 content of the nucleotide sequence is about 95% or greater. In some embodiments, the GC3 content of the nucleotide sequence is about 96% or greater. In some embodiments, the GC3 content of the nucleotide sequence is about 97% or greater. In some embodiments, the GC3 content of the nucleotide sequence is about 98% or greater. In some embodiments, the GC3 content of the nucleotide sequence is about 99% or greater. In some embodiments, the GC3 content of the nucleotide sequence is about 100%. Certain embodiments are directed to a nucleic acid comprising the nucleotide sequence of SEQ ID NO: 11.

In some embodiments, the nucleotide sequence is 60% or more identical to SEQ ID NO: 10. In some embodiments, the nucleotide sequence is 61% or more identical to SEQ ID NO: 10. In some embodiments, the nucleotide sequence is 62% or more identical to SEQ ID NO: 10. In some embodiments, the nucleotide sequence is 63% or more identical to SEQ ID NO: 10. In some embodiments, the nucleotide sequence is 64% or more identical to SEQ ID NO: 10. In some embodiments, the nucleotide sequence is 65% or more identical to SEQ ID NO: 10. In some embodiments, the nucleotide sequence is 66% or more identical to SEQ ID NO: 10. In some embodiments, the nucleotide sequence is 67% or more identical to SEQ ID NO: 10. In some embodiments, the nucleotide sequence is 68% or more identical to SEQ ID NO: 10. In some embodiments, the nucleotide sequence is 69% or more identical to SEQ ID NO: 10. In some embodiments, the nucleotide sequence is 70% or more identical to SEQ ID NO: 10. In some embodiments, the nucleotide sequence is 71% or more identical to SEQ ID NO: 10. In some embodiments, the nucleotide sequence is 72% or more identical to SEQ ID NO: 10. In some embodiments, the nucleotide sequence is 73% or more identical to SEQ ID NO: 10. In some embodiments, the nucleotide sequence is 74% or more identical to SEQ ID NO: 10. In some embodiments, the nucleotide sequence is 75% or more identical to SEQ ID NO: 10. In some embodiments, the nucleotide sequence is 76% or more identical to SEQ ID NO: 10. In some embodiments, the nucleotide sequence is 77% or more identical to SEQ ID NO: 10. In some embodiments, the nucleotide sequence is 78% or more identical to SEQ ID NO: 10. In some embodiments, the nucleotide sequence is 79% or more identical to SEQ ID NO: 10. In some embodiments, the nucleotide sequence is 80% or more identical to SEQ ID NO: 10. In some embodiments, the nucleotide sequence is 85% or more identical to SEQ ID NO: 10. In some embodiments, the nucleotide sequence is 90% or more identical to SEQ ID NO: 10. In some embodiments, the nucleotide sequence is 95% or more identical to SEQ ID NO: 10. In some embodiments, the nucleotide sequence is 99% or more identical to SEQ ID NO: 10.

In some embodiments, the nucleic acid further comprises a cis-regulatory element in functional association with the nucleotide sequence. Sometimes the cis-regulatory element comprises a post transcriptional processing element. At times, the post transcriptional regulatory element is from woodchuck hepatitis virus. In some embodiments the nucleic acid is in an expression vector. In some cases, the nucleic acid encodes a protein comprising a tag.

Also provided is a cell comprising any of the above nucleic acids comprising any of the corresponding embodiments. Sometimes the cell comprises the nucleotide sequence integrated into cellular DNA. Sometimes the cell secretes the soluble viral fusion protein. Sometimes the viral fusion protein is retained in the cell. Sometimes the viral fusion protein is retained in the cell membrane. In some cases, the cell expresses at least 1 microgram of the protein per milliliter of cells. In some cases, the cell expresses at least 2 micrograms of the protein per milliliter of cells. In some cases, the cell expresses at least 3 micrograms of the protein per milliliter of cells. In some cases, the cell expresses at least 4 micrograms of the protein per milliliter of cells. In some cases, the cell expresses at least 5 micrograms of the protein per milliliter of cells. In some cases, the cell expresses at least 6 micrograms of the protein per milliliter of cells. In some cases, the cell expresses at least 7 micrograms of the protein per milliliter of cells. In some cases, the cell expresses at least 8 micrograms of the protein per milliliter of cells. In some cases, the cell expresses at least 9 micrograms of the protein per milliliter of cells. In some cases, the cell expresses at least 10 micrograms of the protein per milliliter of cells. In some cases, the cell expresses at least 100 micrograms of the protein per milliliter of cells. In some cases, the cell expresses at least 200 micrograms of the protein per milliliter of cells. In some cases, the cell expresses at least 300 micrograms of the protein per milliliter of cells. In some cases, the cell expresses at least 400 micrograms of the protein per milliliter of cells. In some cases, the cell expresses at least 500 micrograms of the protein per milliliter of cells. In some cases, the cell expresses at least 600 micrograms of the protein per milliliter of cells. In some cases, the cell expresses at least 700 micrograms of the protein per milliliter of cells. In some cases, the cell expresses at least 800 micrograms of the protein per milliliter of cells. In some cases, the cell expresses at least 900 micrograms of the protein per milliliter of cells. In some cases, the cell expresses at least 1 milligram of the protein per milliliter of cells. In some cases, the cell expresses about 1.3 milligrams or more of the protein per milliliter of cells. In some cases, the cell expresses about 1.6 milligrams or more of the protein per milliliter of cells. In some cases, the cell expresses at least 2 milligrams of the protein per milliliter of cells.

In some cases, the cell is a mammalian cell. At times, the cell is a non-adherent cell. Sometimes the cell is a CHO cell or CHO-derived cell. Sometimes the cell is a CAT-S cell. Sometimes the cell is a CHO-S cell. In some cases, the cell is a Vero cell. In some cases, the cell is a MRC-5 cell. In some cases, the cell is a BSR-T7 cell.

In some cases, the cell synthesizes nucleic acid encoding the viral fusion protein in the nucleus.

Also provided in certain embodiments is a method for expressing a soluble viral fusion protein, comprising contacting a plurality of cells comprising any of the nucleotide sequences and their corresponding embodiments provided above to conditions under which the protein is produced. In some embodiments of the method, the nucleotide sequence is in an expression vector in the cells. Sometimes the nucleotide sequence is in cellular DNA of the cells.

In some embodiments of the method, the cell is a mammalian cell. The cell in certain embodiments is a non-adherent cell. Sometimes the cell is a CHO cell or CHO-derived cell. Sometimes the cell is a CAT-S cell. Sometimes the cell is a CHO-S cell. In some cases, the cell is a Vero cell. In some cases, the cell is a MRC-5 cell. In some cases, the cell is a BSR-T7 cell.

In some embodiments of the method, the cells secrete the protein. In some embodiments of the method, the protein is retained in the cell. In some embodiments of the method, the protein is retained in the cell membrane. In some cases, the cell expresses at least 1 microgram of the protein per milliliter of cells. In some cases, the cell expresses at least 2 micrograms of the protein per milliliter of cells. In some cases, the cell expresses at least 3 micrograms of the protein per milliliter of cells. In some cases, the cell expresses at least 4 micrograms of the protein per milliliter of cells. In some cases, the cell expresses at least 5 micrograms of the protein per milliliter of cells. In some cases, the cell expresses at least 6 micrograms of the protein per milliliter of cells. In some cases, the cell expresses at least 7 micrograms of the protein per milliliter of cells. In some cases, the cell expresses at least 8 micrograms of the protein per milliliter of cells. In some cases, the cell expresses at least 9 micrograms of the protein per milliliter of cells. In some cases, the cell expresses at least 10 micrograms of the protein per milliliter of cells. In some cases, the cell expresses at least 100 micrograms of the protein per milliliter of cells. In some cases, the cell expresses at least 200 micrograms of the protein per milliliter of cells. In some cases, the cell expresses at least 300 micrograms of the protein per milliliter of cells. In some cases, the cell expresses at least 400 micrograms of the protein per milliliter of cells. In some cases, the cell expresses at least 500 micrograms of the protein per milliliter of cells. In some cases, the cell expresses at least 600 micrograms of the protein per milliliter of cells. In some cases, the cell expresses at least 700 micrograms of the protein per milliliter of cells. In some cases, the cell expresses at least 800 micrograms of the protein per milliliter of cells. In some cases, the cell expresses at least 900 micrograms of the protein per milliliter of cells. In some cases, the cell expresses at least 1 milligram of the protein per milliliter of cells. In some cases, the cell expresses about 1.3 milligrams or more of the protein per milliliter of cells. In some cases, the cell expresses about 1.6 milligrams or more of the protein per milliliter of cells. In some cases, the cell expresses about 2 milligrams or more of the protein per milliliter of cells.

In some embodiments of the method, the protein is produced for 5 or more days. In some embodiments of the method, the protein is produced for 6 or more days. In some embodiments of the method, the protein is produced for 7 or more days. In some embodiments of the method, the protein is produced for 8 or more days. In some embodiments of the method, the protein is produced for 10 or more days.

In some embodiments of the method, the cells are cultured under animal product-free culture conditions. The method sometimes further comprises determining the amount of protein produced by the cells. In some cases, the method further comprises isolating the protein.

In some embodiments of the method, the cell synthesizes the nucleic acid encoding the viral fusion protein in the nucleus. The nucleic acid is sometimes introduced into the cell nucleus. In certain embodiments, the nucleic acid is introduced into the cell nucleus by nucleotransfection.

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1A illustrates a schematic of recombinant RSV-F protein expression cassettes. FIG. 1B provides a comparison of RSV-F protein GC content for constructs F_{A2}, F_{OPT} and F_{GC}.

FIG. 2 provides an illustration of the pCLD550v4 synthetic MCS-SV40pA expression vector.

FIG. 3 provides an illustration of the pCLD550v4 synthetic MCS-SV40pA expression vector with the soluble GC3 construct cloned into the Fsel and Sbfl restriction sites.

FIG. 4 shows recombinant RSV-F protein expression is improved by increased GC abundance. Western blots are presented for triplicate RSV-F protein of lysates from BSR-T7, MRC-5, and Vero cell lines at 36 h post-transfection with pCMVscript RSV-F protein expression vectors. The F_{A2} (wild-type), Fₒₚₜ (codon optimized), and F_{GC} (GC-enriched) constructs were tested for each cell type. Protein loading was normalized to beta-actin.

FIG. 5 shows expression of RSV-F across cell lines transfected with wild-type (F), codon optimized (Fₒₚₜ), and GC-enriched (F_{xgc}) sequences. Three nucleotide versions encoding identical RSV-F protein were constructed in the pCMVscript expression vector and transfected into BSRT7, MRC-5, and Vero cells. While levels of RSV-F (48 kDa) were not detected in any of the cells transfected with the wild-type sequence, the protein was expressed at low to moderate levels in MRC-5 and BSRT7 cells, respectively, for the codon optimized sequence. RSV-F protein levels were maximal in all cell lines when the GC-enriched sequence was transfected.

FIG. 6 shows recombinant RSV-F protein expression is improved by increased GC abundance and enhanced by the presence of WPRE. A Western blot is presented for RSV-F protein of lysates from 293F cells transfected with pEBNA RSV-F protein expression vectors. The lysates were first diluted to normalize for protein loading based on beta-actin. These normalized lysates were then further diluted, where the lysates that had greater levels of RSV-F protein (Fₒₚₜ, F_{opt-WPRE}, F_{GC}, and F_{GC-WPRE}) were diluted 10-fold more than those that had less RSV-F protein (F_{Long}, F_{Long-WPRE}, F_{A2}, and F_{A2-WPRE}) prior to loading for ease of comparison, as noted at the top of the blot. A Western blot carried out in the same manner of lysates from infected HEp-2 cells is included for comparison. An asterisk is used to mark the faint bands observed at 48 kDa. The Western blot is representative of at least three replicates of each experiment.

FIG. 7A and FIG. 7B show increased GC abundance does not improve recombinant RSV-F protein expression from a recombinant b/hPIV3 cytoplasmic expression vector. FIG. 7A provides a schematic of the b/hPIV3+RSV-F expression vector. FIG. 7B shows a Western blot for RSV-F protein of lysates from Vero cells infected with b/hPIV3-RSV-F recombinant viruses infected at an MOI of 0.1 and harvested at 48 h post infection. Protein loading was normalized to PIV3 HN gene expression. Western blot is representative of at least three replicates of each experiment.

FIG. 8A and FIG. 8B show premature polyadenylation occurs during recombinant RSV-F protein expression. FIG. 8A provides a summary of polyadenylation signal sequences found in each RSV-F protein sequence. FIG. 8B shows 1% Agarose gel analysis of 3′ RACE RT-PCR performed on RNA purified from 293F cells transfected with the pEBNA RSV-F protein expression vectors. For ease of comparison, 2 microliters of wild-type cDNA was used in the PCR, whereas 0.5 microliters of Fₒₚₜ and F_{GC} cDNA was used in the PCR. Minus-RT step controls are included in the bottom half of the gel. Full-length transcripts should be approximately 1,700 nucleotides in length as indicated.

FIG. 9 shows increased RSV-F protein expression correlates with increased syncytium formation upon transfection. Microscopic images of 293Ad cells 24 h post-transfection with pEBNA RSV-F protein expression vectors. The pEBNA vector encoding the green fluorescent protein (gfp) was included to approximate transfection efficiency. Images are representative of at least three replicates of each experiment.

FIG. 10 shows a CAT-S test of sRSV-F constructs. Western blots of CAT-S transfectant supernatants are presented. Supernatants were normalized to 2×10⁶ viable cells per mL for each cell population. Protein was visualized with either Motavizumab or goat anti-RSV as indicated. The following samples were loaded in each numbered well: 1. wild-type; 2. codon optimized; 3. GC rich; 4. HL2 (medium GC3); 5. GH5; 6. gfp.

FIG. 11 shows a CAT-S test of sRSV-F constructs. 12% reducing SDS-PAGE Western blots with Motavizumab, at a dilution of 1/10,000, and anti-humanHRP, at a dilution of 1/1000, or Goat anti-RSV are presented. Supernatants were normalized to 2×10⁶ viable cells per mL for each cell population. Protein was visualized with either Motavizumab or goat anti-RSV as indicated. The following samples were loaded in each numbered well: 1. MAGIC MARK; 2. SEEBLUE PLUS; 3. wild-type; 4. codon optimized; 5. GC rich; 6. HL2 (medium GC3); 7. GH5; 8. gfp. Three different exposures of the blot are presented.

FIG. 12 shows a CHO-S test of sRSV-F constructs. Western blots of CHO-S transfectants are presented. Supernatants were normalized to 2×10⁶ viable cells per mL for each cell population. Protein was visualized with either Motavizumab or goat anti-RSV as indicated. The following samples were loaded in each numbered well: 1. wild-type; 2. codon optimized; 3. GC rich; 4. HL2 (medium GC3); 5. GH5; 6. gfp; 7. sRSV-F clone 10 (CAT-S produced sRSV-F protein used as a control).

FIG. 13 shows a CHO-S test of sRSV-F constructs. Western blots of CHO-S transfectants are presented. Protein was visualized with Motavizumab. The following samples were loaded in each numbered well: 1. MAGIC MARK; 2. SEEBLUE PLUS 2; 3. wild-type; 4. codon optimized; 5. GC rich; 6. HL2 (medium GC3); 7. GH5; 8. gfp; 9. sRSV-F clone 10 (CAT-S produced sRSV-F protein used as a control). Exposures of the blot are presented at 2 minutes, 5 minutes and 10 minutes.

FIG. 14 shows sRSV-F production levels from CAT-S and CHO-S cells by ELISA. ELISA titers of sRSV-F protein are indicated for each transfectant supernatant. Supernatants were normalized to 2×10⁶ viable cells per mL for each cell population and analyzed by quantitative ELISA for sRSV-F quantitation.

FIG. 15 shows FACS analysis of sRSV-F expression in CAT-S (left) and CHO-S (right) cells. The number of cells exhibiting various levels of sRSV-F protein is presented for each transfectant.

FIG. 16A presents FACS analysis of sRSV-F expression in CAT-S cells. Mean fluorescence intensity is provided for each transfectant. The scale for mean fluorescence intensity for CAT-S cells is 150 to 750. FIG. 16B presents FACS analysis of sRSV-F expression in CHO-S cells. Mean fluorescence intensity is provided for each transfectant. The scale for mean fluorescence intensity for CHO-S cells is 0 to 300.

FIG. 17 presents FACS analysis of sRSV-F expression in CAT-S and CHO-S cells. The data in the graphs presented in FIG. 16A and FIG. 16B has been merged in FIG. 17 in order to provide a side-by-side comparison of fluorescence intensities for each cell type.

FIG. 18 shows determination of sRSV-F protein levels in CAT-S parental clones by quantitative ELISA. Culture media from initial 96-well plate CAT-S colonies transfected with variable forms of sRSV-F nucleotide sequence were screened for recombinant protein expression using a quantitative sandwich ELISA. Initial screening indicated highest levels of sRSV-F were produced by cells containing the GC-enriched coding sequence that was greater than 10-fold better than that exhibited by either codon optimized or wild-type sequence.

FIG. 19 shows production of sRSV-F by the uppermost CAT-S (GC-enriched) parental clones. Culture media from initial shake flask overgrowth cultures of the top four CAT-S parental clones were screened for recombinant sRSV-F protein expression using a quantitative sandwich ELISA. Results indicate peak production up to 90 micrograms/mL sRSV-F can be generated by these cells, which is at least six fold higher than previously achieved.

FIG. 20 shows Anti-RSV-F Western blot analysis of the uppermost CAT-S (GC-enriched) parental clones. Culture media from initial shake flask overgrowth cultures of the top four CAT-S parental clones were run under non-reduced and reduced conditions via SDS-PAGE and detected with goat anti-RSV polyclonal antibody. Levels of sRSV-F increased dramatically over the course of shake flask culture and high molecular weight aggregates of the protein were apparent under non-reducing conditions.

FIG. 21 shows a CAT-S test of hPIV3-F constructs. Western blots of CAT-S transfectant lysates are presented. Lysates were generated from three independent wells inoculated with cells from identical transfection mixtures. Protein was visualized with polyclonal anti-PIV3, anti-HIS, or anti-C-terminal HIS, as indicated.

FIG. 22 presents a table of the genetic code. Table is modified from http address www.mun.ca/biology/scarr/MGA2_{—}03-20.html, incorporated by reference herein.

FIG. 23 shows sRSV-F protein levels in CAT-S parental clones as determined by quantitative ELISA. Culture media from CAT-S clones transfected with variable forms of sRSV-F nucleotide sequence were screened by quantitative ELISA during the scale-up process. Clones highlighted in grey were not selected for further growth. Iterative screening indicated highest levels of sRSV-F were produced by cells containing the GC-enriched coding sequence, which was generally greater than 10 fold better than that exhibited by codon-optimized clones.

### DETAILED DESCRIPTION

Provided herein are recombinantly expressed viral fusion proteins, nucleic acid that encodes them, cells that contain the nucleotide sequences of such nucleic acid, and methods for producing the fusion proteins from such cells. Expressed recombinant viral fusion proteins can be used for structural studies of viral fusion proteins and studies of their membrane fusion activity. Since viral fusion proteins are glycoproteins often found on the surface of certain viruses and thus easily accessible to immunosurveillance, these proteins can serve as targets for neutralizing antibodies. Viral fusion proteins often are less variable than other glycoproteins found on the viral surface, and expression of viral fusion proteins can be useful for the development of vaccines against certain viruses that express these glycoproteins. The forgoing applications can depend on an adequate expression level of a particular viral fusion protein, which is provided by the compositions and methods described herein.

### Viral Fusion Glycoproteins

Viral fusion glycoproteins mediate entry of a virus into a host cell during viral infection via membrane fusion induction. Provided herein are recombinantly expressed viral fusion proteins. As used herein, “viral fusion protein” refers to any viral fusion protein, including but not limited to, a native viral fusion protein, a recombinant viral fusion protein, a synthetically produced viral fusion protein, and a viral fusion protein extracted from cells. As used herein, “native viral fusion protein” refers to a viral fusion protein encoded by a naturally occurring viral gene or viral RNA that is present in nature. As used herein, the term “recombinant viral fusion protein” refers to a viral fusion protein derived from an engineered nucleotide sequence and produced in an in vitro and/or in vivo expression system. Alternative names that are used interchangeably for viral fusion protein include “viral fusion glycoprotein” and “F protein.” Viral fusion proteins include related proteins from different viruses and viral strains including, but not limited to viral strains of human and non-human categorization. Viral fusion proteins can be related by amino acid sequence, protein structure, and/or function. Viral fusion proteins include members assigned to all classes of viral fusion proteins, including, but not limited to, members assigned to type I and type II viral fusion proteins.

Viral fusion proteins include precursor (F₀) proteins, with or without a signal peptide, and activated and/or mature fragments, including F1 and F2 subunits. As used herein, the terms “mature” and “activated” refer to viral fusion proteins that have been converted from a precursor protein to the mature fusion protein by host proteases. Typically, activated viral fusion proteins are composed of a membrane-anchored and a membrane-distal subunit, which are named F1 and F2, respectively, in certain types of viruses. The active F1 and F2 subunits are often linked together via a disulfide bond. Viral fusion proteins can be of any tertiary structure such as, for example, monomers, dimers, trimers or hexamers.

Recombinant viral fusion proteins can be of any length and can include any modification, fusion, mutation, replacement, amino acid change, deletion, insertion or addition, provided that the viral fusion protein retains at least one functional and/or antigenic characteristic typical of a native, full-length, mature viral fusion protein counterpart. For example, a recombinant viral fusion protein can have 1, 2, 3, 4, 5, 6, 7, 8, 9, 10, 11, 12, 13, 14, 15, 16, 17, 18, 19, 20 or more amino acid modifications provided that the viral fusion protein retains at least one functional and/or antigenic characteristic typical of a native, full-length, mature viral fusion protein counterpart. A functional characteristic of a viral fusion protein, for example, is the ability to induce membrane fusion. The functional characteristic need not be at the same level or activity as exhibited by the native, full-length, mature viral fusion protein counterpart. In some embodiments, a recombinant viral fusion protein retains at least about 20% of the functional characteristic activity of the native, full-length, mature viral fusion protein counterpart (e.g., about 25%, 30%, 35%, 40%, 45%, 50%, 55%, 60%, 65%, 70%, 75%, 80%, 85%, 90%, 95% of the functional characteristic activity of the counterpart).

Recombinant viral fusion protein activity can be assessed by any of the assays for fusion protein function known in the art. For example, cells expressing a viral fusion protein can be monitored via microscopy for cell-to-cell fusion and/or syncytium formation, such as, for example, the cell fusion assay described herein in Example 2. Other assays for membrane fusion include, for example, assays that involve fluorescence/quenching systems or assays that employ synergistic fluorescent components present in separate vesicles, whereupon membrane fusion gives rise to a detectable signal. Such assays are commercially available and include for example, Fluorescence Quenching Assays with ANTS/DPX (Invitrogen) and Fluorescence Enhancement Assays with Tb3+/DPA (Invitrogen).

The ANTS/DPX fluorescence quenching assay can be used to determine membrane fusion activity by viral fusion proteins. This assay is based on the collisional quenching of the polyanionic fluorophore ANTS by the cationic quencher DPX. Separate vesicle populations, one or both expressing a viral fusion protein, are each loaded with ANTS or DPX. Vesicle fusion results in quenching of ANTS fluorescence. Other fluorescence/quencher pairs that can be employed in this type of assay include, but are not limited to, HPTS/DPX, pyrenetetrasulfonic acid/DPX, ANTS/Thallium (Tl+), ANTS/cesium (Cs+), pyranine (HPTS, H348)/Thallium (Tl+), pyranine (HPTS, H348)/cesium (Cs+), pyrenetetrasulfonic acid (P349)/Thallium (Tl+) and pyrenetetrasulfonic acid (P349)/cesium (Cs+).

The Tb3+/dipicolinic acid (DPA) assay also can be used to determine membrane fusion activity by viral fusion proteins. In the Tb3+/DPA assay, separate vesicle populations expressing viral fusion proteins are loaded with TbCl₃ or DPA. Vesicle fusion results in formation of Tb3+/DPA chelates that are approximately 10,000 times more fluorescent than free Tb3+.

Recombinant viral fusion proteins can be further modified, such as by chemical modification, or post-translational modification. Such modifications include, but are not limited to, pegylation, albumination, glycosylation, farnysylation, carboxylation, hydroxylation, hasylation, carbamylation, sulfation, phosphorylation, and other polypeptide modifications known in the art. The viral fusion proteins provided herein can be further modified by modification of the primary amino acid sequence, by deletion, addition, or substitution of one or more amino acids. Recombinant viral fusion proteins can be modified, for example, by post-translational glycosylation. A recombinant viral fusion protein can be fully glycosylated, partially glycosylated, deglycosylated, or non-glycosylated. In some embodiments, a recombinant viral fusion protein (e.g., RSV-F fusion protein, soluble RSV-F fusion protein, soluble hPIV-3 fusion protein) can have a glycosylation profile similar to, substantially identical to, or identical to the glycosylation profile of the native counterpart protein (e.g., Rixon et al., 2002 J. Gen. Virol. 83: 61-66). As used herein the term “glycosylation profile” refers to the amino acid sites on a protein that are glycosylated and the types of glycosylation moiety or moieties at each site. As used herein, a “glycosylation site” refers to an amino position in a polypeptide to which a carbohydrate moiety can be attached. Typically, a glycosylated protein contains one or more amino acid residues, such as asparagine or serine, that can be attached to one or more carbohydrate moieties. As used herein, a “native glycosylation site” refers to an amino acid position, which is attached to a carbohydrate moiety, in a native polypeptide when the native polypeptide is produced in nature. As used herein, a “fully glycosylated” recombinant viral fusion protein is a polypeptide that is glycosylated at all native glycosylation sites in the polypeptide. As used herein, a “deglycosylated” recombinant viral fusion protein has reduced glycosylation compared to the native glycosylated viral fusion protein because it has fewer carbohydrate moieties attached to the polypeptide, such as by virtue of fewer up to all glycosylation sites removed by mutation. Deglycosylated viral fusion proteins also include polypeptides that have one or more carbohydrate moieties removed or partially removed by chemical or enzymatic cleavage. As used herein, a “non-glycosylated” recombinant viral fusion protein is a polypeptide that has no glycosylation (i.e., does not contain carbohydrate moieties attached to glycosylation sites in the protein). A non-glycosylated polypeptide can be produced by a host that does not glycosylate the polypeptide (e.g., prokaryotic host), by elimination of all glycosylation sites (e.g., mutation of glycosylation site amino acids), or elimination of glycosylation moieties from a glycosylated protein.

Recombinant viral fusion glycoproteins can include any of the multiple glycosidic linkages known in the art, including but not limited to N-glycosidic linkages (e.g., GlcNAc-beta-Asn, Glc-beta-Asn, Rha-Asn and Glc-beta-Arg linkages); O-glycosidic linkages (e.g., linkages to Ser, Thr, Tyr, Hyp [hydroxyproline], and Hyl [hydroxylysine]; GalNAc-Ser/Thr, GalNAc-beta-Ser/Thr, Gal-Ser/Thr, Man-Ser/Thr, Fuc-Ser/Thr, Glc-beta-Ser, Pse-Ser/Thr, DiActrideoxyhexose-Ser/Thr, FucNAc-beta-Ser/Thr, Xyl-beta-Ser, Glc-Thr, GlcNAc-Thr, Gal-beta-Hyl, Gal-Hyp, Gal-beta-Hyp, Ara-Hyp Ara-beta-Hyp, GlcNAc-Hyp, Glc-Tyr and Glc-beta-Tyr linkages); C-mannosyl linkages (e.g., mannosyl linkage to C-2 of the Trp through a C—C bond); phosphoglycosyl linkages (e.g., attachment of sugar (e.g., GlcNAc, Man, Xyl, and Fuc) to protein via a phosphodiester bond; GlcNAc-1-P-Ser, Man-1-P-Ser, Xyl-1-P-Ser, Fuc-beta-1-P-Ser linkages); and glypiated linkages (e.g., Man is linked to phosphoethanolamine, which in turn is attached to the terminal carboxyl group of a protein). Extent of glycosylation can be assessed using methods known in the art (e.g., Spiro, Glycobiology 12: 43R-56R (2002)).

Recombinant viral fusion proteins include proteins from any virus or viral strain thereof that expresses a fusion protein. Type I viral fusion proteins are expressed by viruses including, but not limited to, paramyxoviruses, retroviruses, coronaviruses, orthomyxoviruses, and filoviruses. Type II viral fusion proteins are expressed by viruses including, but limited to, alphaviruses and flaviviruses. Type I viral fusion proteins include, for example, paramyxovirus fusion proteins. Paramyxoviruses are negative-sense single-stranded RNA viruses that can cause several human and animal diseases. The paramyxovirus fusion protein projects from the viral envelope surface as a trimer, mediates cell entry by inducing fusion between the viral envelope and the cell membrane, and often requires a neutral pH for fusogenic activity. Viruses of the paramyxovirus family include, but are not limited to, Newcastle disease virus, Hendravirus, Nipahvirus, Measles virus, Mumps virus, Rinderpest virus, Canine distemper virus, phocine distemper virus, Peste des Petits Ruminants virus (PPR), Sendai virus, Human parainfluenza viruses 1, 2, 3 and 4, common cold viruses, Simian parainfluenza virus 5, Menangle virus, Tioman virus, Tuhokovirus 1, 2 and 3, Human respiratory syncytial virus (RSV), Bovine respiratory syncytial virus, Avian pneumovirus, Human metapneumovirus, Fer-de-Lance virus, Nariva virus, Tupaia paramyxovirus, Salem virus, J virus, Mossman virus and Beilong virus.

A recombinant viral fusion protein can be, for example, a respiratory syncytial virus fusion protein (RSV-F). RSV-F proteins can be from any RSV strain or isolate known in the art, including, for example, Human strains such as A2, Long, ATCC VR-26, 19, 6265, E49, E65, B65, RSB89-6256, RSB89-5857, RSB89-6190, and RSB89-6614; or Bovine strains such as ATue51908, 375, and A2Gelfi; or Ovine strains. An RSV-F amino acid sequence can be any RSV-F amino acid sequence provided herein or any sequence with up to 10% variation of the RSV-F amino acid sequences provided herein (e.g., the variant amino acid sequence can be about 90%, 91%, 92%, 93%, 94%, 95%, 96%, 97%, 98% or 99% identical to an amino acid sequence provided herein, or can include 1, 2, 3, 4, 5, 6, 7, 8, 9, 10, 11, 12, 13, 14, 15, 16, 17, 18, 19 or 20 amino acid modifications with respect to an amino acid sequence provided herein). The amino acid sequence of the wild-type RSV-F Human strain A2, for example, is set forth in SEQ ID NO: 2.

A recombinant viral fusion protein can also include, for example, the hPIV3 fusion protein (hPIV3-F). hPIV3-F proteins provided herein can be from any hPIV3 strain or isolate known in the art, including, for example, strains such as 14702, ZHYMgz01, LZ22, Texas/12084/1983, and Wash/47885/57. An hPIV3-F amino acid sequence can be any hPIV3-F amino acid sequence provided herein or any sequence with up to 10% variation of the hPIV3-F amino acid sequences provided herein (e.g., the variant amino acid sequence can be about 90%, 91%, 92%, 93%, 94%, 95%, 96%, 97%, 98% or 99% identical to an amino acid sequence provided herein, or can include 1, 2, 3, 4, 5, 6, 7, 8, 9, 10, 11, 12, 13, 14, 15, 16, 17, 18, 19 or 20 amino acid modifications with respect to an amino acid sequence provided herein). The amino acid sequence of wild-type hPIV3-F strain Texas/12084/1983 for example, is set forth in SEQ ID NO: 9.

### Soluble Viral Fusion Proteins

Also provided herein are recombinant soluble viral fusion proteins. Native, full-length viral fusion proteins typically include a membrane association region, and recombinant soluble viral function proteins generally lack a functional membrane association region, which often is located in the C-terminal region of the native protein. Recombinant soluble viral fusion proteins can be generated by deletion, mutation, or any mode of disruption known in the art, of the functional membrane associated region of a viral fusion protein. For example, any part or all of the membrane association region can be removed or modified provided that the membrane association region is not detectably functional (e.g. region no longer reside in the membrane), and (ii) a certain percent of the membrane association region remains (e.g., about 50% or less remains), is removed (e.g., about 50% or more removed) or is modified (e.g., about 50% or more modified). The extent to which the disrupted membrane associated region no longer confers association of the protein to the plasma membrane can be determined by any technique known in the art that can assess membrane association of proteins. For example, co-immunostaining of the viral fusion protein and a known membrane associated protein can be performed to visualize protein retained in the membrane. Examples of soluble viral fusion proteins are provided herein and include without limitation soluble RSV-F and soluble hPIV3-F. Soluble RSV-F can be generated, for example, by deletion of the 50 amino acid C-terminal transmembrane domain of the RSV-F protein, corresponding to amino acid 525-574 of SEQ ID NO: 2. The amino acid sequence for this example of a soluble RSV-F is set forth in SEQ ID NO: 7. Soluble hPIV3-F can be generated, for example, by the deletion of the C-terminal 51 amino acids, corresponding to amino acids 489-539 of SEQ ID NO: 9. The amino acid sequence for this example of a soluble hPIV3-F is set forth in SEQ ID NO: 12.

Recombinant soluble viral fusion proteins can be generated in any cellular component. Soluble viral fusion proteins can be generated, for example, in the cytoplasm. Recombinant soluble viral fusion proteins also can be expressed in conjunction with a cellular secretory pathway and can be expressed, for example, in the endoplasmic reticulum, Golgi apparatus, plasma membrane and extracellular media. Recombinant soluble viral fusion proteins can accordingly be isolated from various cellular and extra cellular components including the cytoplasm, intracellular vesicles, and/or plasma membrane. Recombinant soluble viral fusion proteins also can be secreted to the extracellular media, and a secreted viral fusion protein can be completely secreted or partially secreted with a remainder of the protein retained in the cell.

### Nucleic Acids

A nucleic acid can be from any source or composition, and can be a deoxyribonucleic acid (DNA), complementary DNA (cDNA), genomic DNA (gDNA), ribonucleic acid (RNA), inhibitory RNA (RNAi), short inhibitory RNA (siRNA), transfer RNA (tRNA) or messenger RNA (mRNA), for example. A nucleic acid can be in any suitable form, including, without limitation, linear, circular, supercoiled, single-stranded, double-stranded, and the like. It is understood that the term “nucleic acid” does not in itself refer to or infer a specific length of the polynucleotide chain, thus polynucleotides and oligonucleotides are also included in the definition. Deoxyribonucleotides include deoxyadenosine, deoxycytidine, deoxyguanosine and deoxythymidine. For RNA, the uracil base is uridine.

A nucleic acid sometimes is a plasmid, phage, autonomously replicating sequence (ARS), centromere, artificial chromosome, yeast artificial chromosome (e.g., YAC) or other nucleic acid able to replicate or be replicated in a host cell. A nucleic acid in some embodiments is from a single chromosome (e.g., a nucleic acid sample may be from one chromosome of a sample obtained from a diploid organism). In certain embodiments a nucleic acid can be from a library or can be obtained from enzymatically digested, sheared or sonicated genomic DNA (e.g., fragmented) from an organism of interest.

In some embodiments, a nucleic acid may be about 5 to about 500 nucleotides or base pairs in length, for example (e.g., about 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230 250, 300, 350, 400, 450, or up to about 500 nucleotides or base pairs in length). In certain embodiments, a nucleic acid can be about 5 to about 300 nucleotides or base pairs in size, or about 5 to about 200 nucleotides or base pairs in size. In certain embodiments, a nucleic acid can be greater than about 200 nucleotides or base pairs in length, and sometimes is about 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 9000, 10000, 11000, 12000, 13000, 14000, 15000, 16000, 17000, 18000, 19000 or 20000 nucleotides or base pairs in length. The term “nucleotides”, as used herein, in reference to the length of nucleic acid chain, refers to a single stranded nucleic acid chain. The term “base pairs”, as used herein, in reference to the length of nucleic acid chain, refers to a double stranded nucleic acid chain.

A nucleic acid can comprise DNA or RNA analogs or modifications (e.g., containing base analogs, modified bases, sugar analogs and/or a non-native backbone and the like). By “modified bases” is meant nucleotide bases other than adenine, guanine, cytosine and uracil at a 1′ position, or their equivalents. A nucleic acid may contain one or more types of modified bases, in some embodiments, and non-limiting examples of base modifications that can be independently introduced into a nucleic acid include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others.

### Vector Nucleic Acids

A nucleic acid often contains a translatable nucleotide sequence, such as a sequence that encodes a viral fusion protein (e.g., soluble viral fusion protein) for example. The translatable nucleotide sequence often is located between a start codon (AUG in ribonucleic acids and ATG in deoxyribonucleic acids) and a stop codon (e.g., UAA (ochre), UAG (amber) or UGA (opal) in ribonucleic acids and TAA, TAG or TGA in deoxyribonucleic acids), and sometimes is referred to herein as an “open reading frame” (ORF). A vector nucleic acid sometimes comprises one or more ORFs. An ORF may be from any suitable source, sometimes from genomic DNA, mRNA, reverse transcribed RNA or complementary DNA (cDNA) or a nucleic acid library comprising one or more of the foregoing, and is from any organism species, such as virus, prokaryote, yeast, fungus, human, insect, nematode, bovine, equine, canine, feline, rat or mouse, for example.

An ORF encoding a viral fusion protein may be inserted or cloned into a vector for replication of the vector, transcription of a portion of the vector (e.g., transcription of the ORF) and/or expression of the protein in a cell. A vector often includes elements that facilitate one or more of cloning an ORF or other nucleic acid element, replication, transcription, translation and selection, for example. Thus, a vector nucleic acid can include one or more or all of the following non-limiting nucleotide elements: one or more promoter elements, one or more 5′ untranslated regions (5′UTRs), one or more regions into which a target nucleotide sequence may be inserted (an “insertion element”), one or more ORFs, one or more 3′ untranslated regions (3′UTRs), and a selection element.

In some embodiments, a vector nucleic acid includes one or more elements that permit insertion of an ORF or other element. Any convenient cloning strategy known in the art may be utilized to incorporate an element, such as an ORF, into a vector nucleic acid. Known methods can be utilized to insert an element into the vector independent of an insertion element, such as (1) cleaving the vector at one or more existing restriction enzyme sites and ligating an element of interest and (2) adding restriction enzyme sites to the vector by hybridizing oligonucleotide primers that include one or more suitable restriction enzyme sites and amplifying by polymerase chain reaction (described in greater detail herein). Other cloning strategies take advantage of one or more insertion sites present or inserted into the vector nucleic acid, such as an oligonucleotide primer hybridization site for PCR, for example, and others described hereafter.

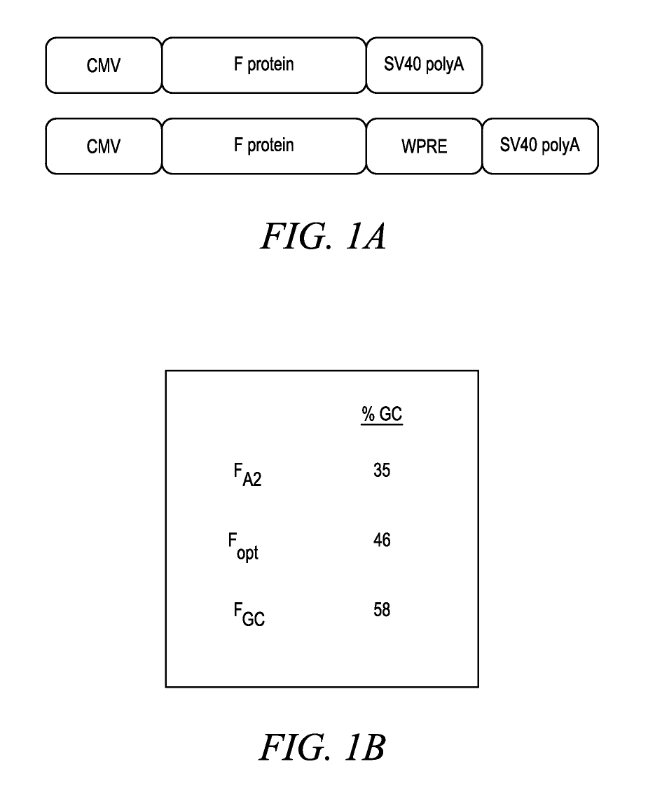

In some embodiments, the vector nucleic acid includes one or more recombinase insertion sites. A recombinase insertion site is a recognition sequence on a nucleic acid molecule that participates in an integration/recombination reaction by recombination proteins. For example, the recombination site for Cre recombinase is loxP, which is a 34 base pair sequence comprised of two 13 base pair inverted repeats (serving as the recombinase binding sites) flanking an 8 base pair core sequence (e.g., FIG. 1 of Sauer, B., Curr. Opin. Biotech. 5:521-527 (1994)). Other examples of recombination sites include attB, attP, attL, and attR sequences, and mutants, fragments, variants and derivatives thereof, which are recognized by the recombination protein λ Int and by the auxiliary proteins integration host factor (IHF), FIS and excisionase (Xis) (e.g., U.S. Pat. Nos. 5,888,732; 6,143,557; 6,171,861; 6,270,969; 6,277,608; and 6,720,140; U.S. patent publication no. 2002-0007051-A1; Landy, Curr. Opin. Biotech. 3:699-707 (1993). All references are incorporated by reference herein.). Examples of recombinase cloning nucleic acids are in Gateway® systems (Invitrogen, California), which include at least one recombination site for cloning a desired nucleic acid molecules in vivo or in vitro. In some embodiments, the system utilizes vectors that contain at least two different site-specific recombination sites, often based on the bacteriophage lambda system (e.g., att1 and att2), and are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway® system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

In certain embodiments, the vector nucleic acid includes one or more topoisomerase insertion sites. A topoisomerase insertion site is a defined nucleotide sequence recognized and bound by a site-specific topoisomerase. For example, the nucleotide sequence 5′-(C/T)CCTT-3′ is a topoisomerase recognition site bound specifically by most poxvirus topoisomerases, including vaccinia virus DNA topoisomerase I. After binding to the recognition sequence, the topoisomerase cleaves the strand at the 3′-most thymidine of the recognition site to produce a nucleotide sequence comprising 5′-(C/T)CCTT-PO₄-TOPO, a complex of the topoisomerase covalently bound to the 3′ phosphate via a tyrosine in the topoisomerase (e.g., U.S. Pat. No. 5,766,891; PCT/US95/16099; and PCT/US98/12372). In comparison, the nucleotide sequence 5′-GCAACTT-3′ is a topoisomerase recognition site for type IA E. coli topoisomerase III. An element to be inserted often is combined with topoisomerase-reacted vector and thereby incorporated into the vector nucleic acid (e.g., http address www.invitrogen.com/downloads/F-13512_Topo_Flyer.pdf; http address at www.invitrogen.com/content/sfs/brochures/710_{—}021849%20_B TOPOCloning_bro.pdf; TOPO TA Cloning® Kit and Zero Blunt® TOPO® Cloning Kit product information).

A vector nucleic acid sometimes contains one or more origin of replication (ORI) elements. In some embodiments, a vector comprises two or more ORIs, where one functions efficiently in one organism (e.g., a bacterium) and another functions efficiently in another organism (e.g., a eukaryote). In some embodiments, an ORI may function efficiently in bacterial cells and another ORI may function efficiently in mammalian cells. A vector nucleic acid also sometimes includes one or more transcription regulation sites.

A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, bird, insect or mammal). The artisan may select appropriate elements for the 5′ UTR based upon the transcription and/or translation system being utilized. A 5′ UTR sometimes comprises one or more of the following elements: translational enhancer sequence, transcription initiation site, transcription factor binding site, translation regulation site, translation initiation site, translation factor binding site, ribosome binding site, replicon, enhancer element, internal ribosome entry site (IRES), and silencer element.

A 3′ UTR may comprise one or more elements endogenous to the nucleotide sequence from which it originates and sometimes includes one or more exogenous elements. A 3′ UTR may originate from any suitable nucleic acid, such as genomic DNA, plasmid DNA, RNA or mRNA, for example, from any suitable organism (e.g., a virus, bacterium, yeast, fungi, plant, insect or mammal). The artisan can select appropriate elements for the 3′ UTR based upon the transcription and/or translation system being utilized. A 3′ UTR sometimes comprises one or more of the following elements: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR often includes a polyadenosine tail and sometimes does not, and if a polyadenosine tail is present, one or more adenosine moieties may be added or deleted from it (e.g., about 5, about 10, about 15, about 20, about 25, about 30, about 35, about 40, about 45 or about 50 adenosine moieties may be added or subtracted).

A vector nucleic acid can include a promoter element that can be placed in functional association with one or more ORFs. A promoter element typically is required for DNA synthesis and/or RNA synthesis. A promoter often interacts with a RNA polymerase. A polymerase is an enzyme that catalyzes synthesis of nucleic acids using a preexisting nucleic acid template. When the template is a DNA template, an RNA molecule is transcribed before protein is synthesized. Enzymes having suitable polymerase activity include any polymerase that is active in the chosen system with the chosen vector to synthesize protein. Non-limiting examples of polymerases include RNA polymerase II, SP6 RNA polymerase, T3 RNA polymerase, T7 RNA polymerase, RNA polymerase III and phage derived RNA polymerases. These and other polymerases are known and nucleic acid sequences with which they interact are known. Such sequences are readily accessed by the artisan, such as by searching one or more public or private databases, for example, and the sequences are readily adapted to vector nucleic acids described herein. Non-limiting examples of promoters are inducible, repressible, non-inducible, constitutive, strong and weak promoters, and can be obtained from any suitable organism (e.g., virus, prokaryote, yeast, fungus, mammal). A promoter element sometimes is placed directly adjacent to an ORF, and sometimes is spaced from the ORF by one or more nucleotides in the vector nucleic acid, provided that the promoter can functionally drive the production of transcript RNA from the ORF.

Any suitable promoter can be used in a nucleic acid vector described herein, so long as the promoter provides levels of transcript production suitable for high levels of protein production in transfected cell lines. Non-limiting examples of promoters suitable for use with nucleic acid vectors described herein include, human CMV major intermediate early gene (hCMV-MIE) promoter, SV40 promoter, CaMV promoter, MMTV promoter, Pol I promoters, Pol II promoters, Pol III promoters, and the like.

A vector nucleic acid often includes one or more selection elements. Selection elements often are utilized using known processes to determine whether a vector nucleic acid is included in a cell. In some embodiments, a vector nucleic acid includes two or more selection elements, where one functions efficiently in cells of one organism (e.g., prokaryote) and another functions efficiently in cells of another organism (e.g., mammal). Examples of selection elements include, but are not limited to, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic (e.g., Diphtheria toxin) or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like).

A stop codon at the end of an ORF sometimes is modified to another stop codon, such as an amber stop codon described above. In some embodiments, a stop codon is introduced within an ORF, sometimes by insertion or mutation of an existing codon. An ORF comprising a modified terminal stop codon and/or internal stop codon often is translated in a system comprising a suppressor tRNA that recognizes the stop codon. An ORF comprising a stop codon sometimes is translated in a system comprising a suppressor tRNA that incorporates an unnatural amino acid during translation of the target protein or target peptide. Methods for incorporating unnatural amino acids into a target protein or peptide are known, which include, for example, processes utilizing a heterologous tRNA/synthetase pair, where the tRNA recognizes an amber stop codon and is loaded with an unnatural amino acid (e.g., http address www.iupac.org/news/prize/2003/wang.pdf). Unnatural amino acids include but are not limited to D-isomer amino acids, ornithine, diaminobutyric acid, norleucine, pyrylalanine, thienylalanine, naphthylalanine and phenylglycine, alpha and alpha-disubstituted amino acids, N-alkyl amino acids, lactic acid, halide derivatives of natural amino acids such as trifluorotyrosine, p-Cl-phenylalanine, p-Br-phenylalanine, p-I-phenylalanine, L-allyl-glycine, beta-alanine, L-alpha-amino butyric acid, L-gamma-amino butyric acid, L-alpha-amino isobutyric acid, L-epsilon-amino caproic acid, 7-amino heptanoic acid, L-methionine sulfone, L-norleucine, L-norvaline, p-nitro-L-phenylalanine, L-hydroxyproline, L-thioproline, methyl derivatives of phenylalanine (Phe) such as 4-methyl-Phe, pentamethyl-Phe, L-Phe (4-amino), L-Tyr (methyl), L-Phe (4-isopropyl), L-Tic (1,2,3,4-tetrahydroisoquinoline-3-carboxyl acid), L-diaminopropionic acid, L-Phe (4-benzyl), 2,4-diaminobutyric acid, 4-aminobutyric acid (gamma-Abu), 2-amino butyric acid (alpha-Abu), 6-amino hexanoic acid (epsilon-Ahx), 2-amino isobutyric acid (Aib), 3-amino propionic acid, ornithine, norleucine, norvaline, hydroxyproline, sarcosine, citrulline, homocitrulline, cysteic acid, t-butylglycine, t-butylalanine, an amino acid derivitized with a heavy atom or heavy isotope (e.g., Au, deuterium, 15N; useful for synthesizing protein applicable to X-ray crystallographic structural analysis or nuclear magnetic resonance analysis), phenylglycine, cyclohexylalanine, fluoroamino acids, designer amino acids such as beta-methyl amino acids, Ca-methyl amino acids, Na-methyl amino acids, naphthyl alanine, and the like.

### Tags

A nucleic acid (e.g., vector) sometimes comprises a nucleotide sequence adjacent to an ORF (e.g., directly or substantially adjacent) that is translated in conjunction with the ORF and encodes an amino acid tag. The tag-encoding nucleotide sequence can be located 3′ and/or 5′ of an ORF in the nucleic acid, thereby encoding a tag at the C-terminus or N-terminus of the protein or peptide encoded by the ORF. Any tag that does not abrogate transcription and/or translation may be utilized and may be appropriately selected.

A tag sometimes specifically binds a molecule or moiety of a solid phase or a detectable label, for example, thereby having utility for isolating, purifying and/or detecting a protein or peptide encoded by the ORF. In some embodiments, a tag comprises one or more of the following elements: FLAG (e.g., DYKDDDDKG), V5 (e.g., GKPIPNPLLGLDST), c-myc (e.g., EQKLISEEDL), HSV (e.g., QPELAPEDPED), influenza hemagglutinin, HA (e.g., YPYDVPDYA), VSV-G (e.g., YTDIEMNRLGK), bacterial glutathione-S-transferase, maltose binding protein, a streptavidin- or avidin-binding tag (e.g., pcDNA™6 BioEase™ Gateway® Biotinylation System (Invitrogen)), thioredoxin, β-galactosidase, VSV-glycoprotein, a fluorescent protein (e.g., green fluorescent protein and its many color variants), a polylysine or polyarginine sequence, a polyhistidine sequence (e.g., His6) or other sequence that chelates a metal (e.g., cobalt, zinc, nickel, copper), and/or a cysteine-rich sequence that binds to an arsenic-containing molecule. In certain embodiments, a cysteine-rich tag comprises the amino acid sequence CC-Xn-CC, wherein X is any amino acid and n is 1 to 3, and the cysteine-rich sequence sometimes is CCPGCC. In certain embodiments, the tag comprises a cysteine-rich element and a polyhistidine element (e.g., CCPGCC and His6).

A tag often conveniently binds to a binding partner. For example, some tags bind to an antibody (e.g., FLAG) and sometimes specifically bind to a small molecule. For example, a polyhistidine tag specifically chelates a bivalent metal, such as copper, zinc, nickel, and cobalt; a polylysine or polyarginine tag specifically binds to a zinc finger; a glutathione S-transferase tag binds to glutathione; and a cysteine-rich tag specifically binds to an arsenic-containing molecule. Arsenic-containing molecules include LUMIO™ agents (Invitrogen, California), such as FlAsH™ ([4′,5′-bis(1,3,2-dithioarsolan-2-yl)fluorescein-(1,2-ethanedithiol)2]) and ReAsH reagents (e.g., U.S. Pat. No. 5,932,474 to Tsien et al., entitled “Target Sequences for Synthetic Molecules;” U.S. Pat. No. 6,054,271 to Tsien et al., entitled “Methods of Using Synthetic Molecules and Target Sequences;” U.S. Pat. Nos. 6,451,569 and 6,008,378; published U.S. Patent Application 2003/0083373, and published PCT Patent Application WO 99/21013, all to Tsien et al. and all entitled “Synthetic Molecules that Specifically React with Target Sequences”, all incorporated by reference for all disclosure of arsenic-containing dyes, tetracys sequence tags, and protein detection). Such antibodies and small molecules sometimes are linked to a solid phase for convenient isolation of the recombinant polypeptide, as described in greater detail hereafter. A tag sometimes is a polypeptide different than the viral fusion protein, such as a polypeptide that facilitates purification of the viral fusion protein. Non-limiting examples of such polypeptides include glutathione binding protein and maltose binding protein.

A tag sometimes comprises a sequence that localizes a translated protein or peptide to a component in a transcription and/or translation system, which is referred to as a “signal sequence” or “localization signal sequence” herein. A signal sequence often is incorporated at the N-terminus of a target protein or target peptide, and sometimes is incorporated at the C-terminus. Examples of signal sequences are known in the art, are readily incorporated into a nucleic acid, and often are selected according to the cells from a viral fusion protein is prepared. A signal sequence in some embodiments localizes a translated protein or peptide to a cell membrane. Examples of signal sequences include, but are not limited to, a nucleus targeting signal (e.g., steroid receptor sequence and N-terminal sequence of SV40 virus large T antigen); mitochondia targeting signal (e.g., amino acid sequence that forms an amphipathic helix); peroxisome targeting signal (e.g., C-terminal sequence in YFG from S. cerevisiae); and a secretion signal (e.g., N-terminal sequences from invertase, mating factor alpha, PHO5 and SUC2 in S. cerevisiae; multiple N-terminal sequences of B. subtilis proteins (e.g., Tjalsma et al., Microbiol. Molec. Biol. Rev. 64: 515-547 (2000)); alpha amylase signal sequence (e.g., U.S. Pat. No. 6,288,302); pectate lyase signal sequence (e.g., U.S. Pat. No. 5,846,818); precollagen signal sequence (e.g., U.S. Pat. No. 5,712,114); OmpA signal sequence (e.g., U.S. Pat. No. 5,470,719); Iam beta signal sequence (e.g., U.S. Pat. No. 5,389,529); B. brevis signal sequence (e.g., U.S. Pat. No. 5,232,841); and P. pastoris signal sequence (e.g., U.S. Pat. No. 5,268,273)).

A tag sometimes is directly adjacent to the amino acid sequence encoded by an ORF (i.e., there is no intervening sequence) and sometimes a tag is substantially adjacent to a the ORF encoded amino acid sequence (e.g., an intervening sequence is present) An intervening sequence sometimes includes a recognition site for a protease, which is useful for cleaving a tag from a target protein or peptide. In some embodiments, the intervening sequence is cleaved by Factor Xa (e.g., recognition site I(E/D)GR), thrombin (e.g., recognition site LVPRGS), enterokinase (e.g., recognition site DDDDK), TEV protease (e.g., recognition site ENLYFQG) or PreScission™ protease (e.g., recognition site LEVLFQGP), for example.

An intervening sequence sometimes is referred to herein as a “linker sequence,” and may be of any suitable length. A linker sequence sometimes is about 1 to about 20 amino acids in length, and sometimes about 5 to about 10 amino acids in length. The artisan may select the linker length to substantially preserve target protein or peptide function (e.g., a tag may reduce target protein or peptide function unless separated by a linker), to enhance disassociation of a tag from a target protein or peptide when a protease cleavage site is present (e.g., cleavage may be enhanced when a linker is present), and to enhance interaction of a tag/target protein product with a solid phase. A linker can be of any suitable amino acid content, and often comprises a higher proportion of amino acids having relatively short side chains (e.g., glycine, alanine, serine and threonine).

A nucleic acid sometimes includes a stop codon between a tag element and an insertion element or ORF, which can be useful for translating an ORF with or without the tag. Mutant tRNA molecules that recognize stop codons (described above) suppress translation termination and thereby are designated “suppressor tRNAs.” Suppressor tRNAs can result in the insertion of amino acids and continuation of translation past stop codons (e.g., U.S. Patent Application No. 60/587,583, filed Jul. 14, 2004, entitled “Production of Fusion Proteins by Cell-Free Protein Synthesis,”; Eggertsson, et al., (1988) Microbiological Review 52(3):354-374, and Engleerg-Kukla, et al. (1996) in Escherichia coli and Salmonella Cellular and Molecular Biology, Chapter 60, pps 909-921, Neidhardt, et al. eds., ASM Press, Washington, D.C.). A number of suppressor tRNAs are known, including, but not limited to, supE, supP, supD, supF and supZ suppressors, which suppress the termination of translation of the amber stop codon; supB, gIT, supL, supN, supC and supM suppressors, which suppress the function of the ochre stop codon and glyT, trpT and Su-9 suppressors, which suppress the function of the opal stop codon. In general, suppressor tRNAs contain one or more mutations in the anti-codon loop of the tRNA that allows the tRNA to base pair with a codon that ordinarily functions as a stop codon. The mutant tRNA is charged with its cognate amino acid residue and the cognate amino acid residue is inserted into the translating polypeptide when the stop codon is encountered. Mutations that enhance the efficiency of termination suppressors (i.e., increase stop codon read-through) have been identified. These include, but are not limited to, mutations in the uar gene (also known as the prfA gene), mutations in the ups gene, mutations in the sueA, sueB and sueC genes, mutations in the rpsD (ramA) and rpsE (spcA) genes and mutations in the rpIL gene.

Thus, a nucleic acid comprising a stop codon located between an ORF and a tag can yield a translated ORF alone when no suppressor tRNA is present in the translation system, and can yield a translated ORF-tag fusion when a suppressor tRNA is present in the system. In some embodiments, the stop codon is located 3′ of an insertion element or ORF and 5′ of a tag, and the stop codon sometimes is an amber codon. Suppressor tRNA sometimes are within a cell-free extract (e.g., the cell-free extract is prepared from cells that produce the suppressor tRNA), sometimes are added to the cell-free extract as isolated molecules, and sometimes are added to a cell-free extract as part of another extract. A provided suppressor tRNA sometimes is loaded with one of the twenty naturally occurring amino acids or an unnatural amino acid (described herein). Suppressor tRNA can be generated in cells transfected with a nucleic acid encoding the tRNA (e.g., a replication incompetent adenovirus containing the human tRNA-Ser suppressor gene can be transfected into cells). Vectors for synthesizing suppressor tRNA and for translating ORFs with or without a tag are available to the artisan (e.g., Tag-On-Demand™ kit (Invitrogen Corporation, California); Tag-On-Demand™ Suppressor Supernatant Instruction Manual, Version B, 6 Jun. 2003, at http address www.invitrogen.com/content/sfs/manuals/tagondemand_supernatant_man.pdf; Tag-On-Demand™ Gateway® Vector Instruction Manual, Version B, 20 Jun., 2003 at http address www.invitrogen.com/content/sfs/manuals/tagondemand_vectors_man.pdf; and Capone et al., Amber, ochre and opal suppressor tRNA genes derived from a human serine tRNA gene. EMBO J. 4:213, 1985).

### Nucleotide and Amino Acid Sequence Comparisons

The term “identical” as used herein refers to two or more nucleotide sequences having substantially the same nucleotide sequence when compared to each other. The term “identical” as used herein also refers to two or more amino acid sequences having substantially the same amino acid sequence when compared to each other. One test for determining whether two nucleotide sequences or amino acids sequences are substantially identical is to determine the percent of identical nucleotide sequences or amino acid sequences shared.

Calculations of sequence identity can be performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, or 100% of the length of the reference sequence. The nucleotides or amino acids at corresponding nucleotide or polypeptide positions, respectively, are then compared among the two aligned sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address www.gcg.com), using either a Blossum 62 matrix or a PAM250 matrix. A set of parameters often used with a Blossum 62 scoring matrix includes a gap open penalty of 12, a gap extend penalty of 4, and a frameshift gap penalty of 5. Percent identity between two nucleotide sequences can be determined using the GAP program in the GCG software package (available at http address www.gcg.com), using a NWSgapdna.CMP matrix and a gap weight of 60 and a length weight of 4.

Another manner for determining whether two nucleic acids are substantially identical is to assess whether a polynucleotide homologous to one nucleic acid will hybridize to the other nucleic acid under stringent conditions. As used herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

### Codon Modification and GC Content

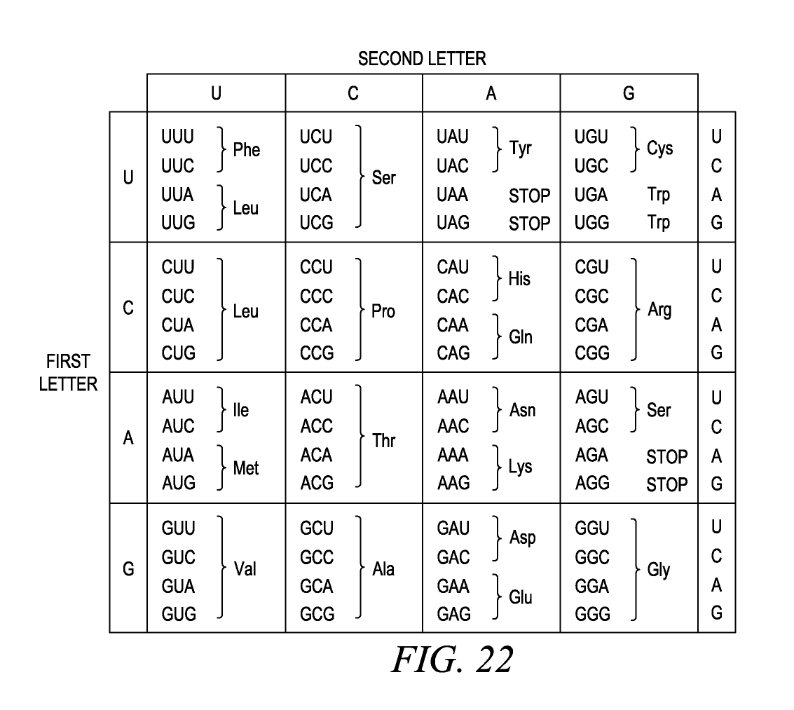

A nucleic acids encoding a viral fusion protein provided herein can be modified by changing one or more nucleotide bases within one or more codons throughout the nucleotide sequence. As used herein, “nucleotide base” refers to any of the four deoxyribonucleic acid bases, adenine (A), guanine (G), cytosine (C), and thymine (T) or any of the four ribonucleic acid bases, adenine (A), guanine (G), cytosine (C), and uracil (U). As used herein, “codon” refers to a series of three nucleotide bases that code for a particular amino acid. The genetic code is presented in FIG. 22, where substantially all possibilities of three nucleotide base combinations are assembled and, in most cases, assigned to a particular amino acid. Generally, each amino acid can be encoded by one or more codons. Table 1 below presents substantially all codon possibilities for each amino acid.

TABLE 1
DNA Codon Table

| Amino | |
|---|---|
| Acid | DNA Codons |
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| START | ATG |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |
| STOP | TAA, TGA, TAG |

Nucleotide sequences provided herein can be modified by changing one or more nucleotide bases within one or more codons such that the amino acid sequence of the encoded viral fusion protein is similar to the amino acid sequence of the protein encoded by the unmodified nucleotide sequence. The encoded viral fusion protein can be 90%, 91%, 92%, 93%, 94%, 95%, 96%, 97%, 98%, 99%, or 100% identical to the protein encoded by the unmodified sequence. In some embodiments, the amino acid sequence encoded by the modified nucleotide sequence is identical to the amino acid sequence encoded by the unmodified nucleotide sequence. As indicated in Table 1, a subset of amino acids and the STOP codon can be encoded by at least two codon possibilities. For example, glutamate can be encoded by GAA or GAG. If a codon for glutamate exists within a nucleic acid sequence as GAA, a nucleotide base change at the third position from an A to a G will lead to a modified codon that still encodes for glutamate. Thus, a particular change in one or more nucleotide bases within a codon can still lead to encoding the same amino acid. This process, in some cases, is referred to herein as codon optimization. Provided herein are examples of nucleotide sequences for RSV-F (set forth in SEQ ID NOs: 16 and 17) that have been modified by changing one or more nucleotide bases within one or more codons whereby the RSV-F amino acid sequence is identical to the amino acid sequence encoded by the unmodified nucleotide sequence (set forth in SEQ ID NO: 2). Also provided herein, for example, are nucleotide sequences for soluble RSV-F (set forth in SEQ ID NOs: 4, 5 and 6) that have been modified by changing one or more nucleotide bases within one or more codons whereby the sRSV-F amino acid sequence is identical to the amino acid sequence encoded by the unmodified nucleotide sequence (set forth in SEQ ID NO: 7). Also provided herein, for example, is a nucleotide sequence for hPIV3-F (set forth in SEQ ID NO: 11) that has been modified by changing one or more nucleotide bases within one or more codons whereby the hPIV3-F amino acid sequence is identical to the amino acid sequence encoded by the unmodified nucleotide sequence (set forth in SEQ ID NO: 12).

The nucleotide sequences provided herein can be modified by changing one or more nucleotide bases within one or more codons such that a) the amino acid sequence of the encoded viral fusion protein is similar or identical to the amino acid sequence of the protein encoded by the unmodified nucleotide sequence; and b) the combined percent of guanines and cytosines (% GC) is increased in the modified nucleotide sequence compared to the unmodified nucleotide sequence. For example, the % GC can be about 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, or 99% or more. As indicated in Table 1, nucleotide base changes at the first, second and/or third codon positions can be made whereby an A or a T is changed to a G or a C while preserving the amino acid and/or STOP codon assignment. Provided herein is an example of a nucleotide sequences for RSV-F (set forth in SEQ ID NO: 17) that has been modified by changing one or more nucleotide bases within one or more codons whereby the RSV-F amino acid sequence is identical to the amino acid sequence encoded by the unmodified nucleotide sequence (set forth in SEQ ID NO: 2), and the combined percent of guanines and cytosines (% GC) is increased in the modified nucleotide sequence (58% GC) compared to the unmodified nucleotide sequence (35% GC; set forth in SEQ ID NO: 1). Also provided herein, for example, are nucleotide sequences for soluble RSV-F (e.g., set forth in SEQ ID NOs: 4, 5 and 6) that have been modified by changing one or more nucleotide bases within one or more codons whereby the sRSV-F amino acid sequence is identical to the amino acid sequence encoded by the unmodified nucleotide sequence (set forth in SEQ ID NO: 7), and the combined percent of guanines and cytosines (% GC) is increased in the modified nucleotide sequences (46% GC for SEQ ID NO: 4; 51% GC for SEQ ID NO: 6; 58% GC for SEQ ID NO: 5) compared to the unmodified nucleotide sequence (35% GC; set forth in SEQ ID NO: 3). Also provided herein, for example, is a nucleotide sequence for hPIV3-F (set forth in SEQ ID NO: 11) that has been modified by changing one or more nucleotide bases within one or more codons whereby the hPIV3-F amino acid sequence is identical to the amino acid sequence encoded by the unmodified nucleotide sequence (set forth in SEQ ID NO: 12), and the combined percent of guanines and cytosines (% GC) is increased in the modified nucleotide sequence (60% GC) compared to the unmodified nucleotide sequence (36% GC; set forth in SEQ ID NO: 10).

The nucleotide sequences provided herein can be modified by changing one or more nucleotide bases within one or more codons such that a) the amino acid sequence of the encoded viral fusion protein is similar or identical to the amino acid sequence of the protein encoded by the unmodified nucleotide sequence; b) the combined percent of guanines and cytosines (% GC) is increased in the modified nucleotide sequence compared to the unmodified nucleotide sequence; and c) the overall combined percent of guanines and cytosines at the third nucleotide codon position (% GC3) is increased in the modified nucleotide sequence compared to the unmodified nucleotide sequence. For example, the % GC3 can be about 55, 56, 57, 58, 59, 60, 65, 70, 75, 76, 77, 78, 79, 80, 85, 90, 95, 96, 97, 98, 99, or 100%. As indicated in Table 1, most nucleotide base change possibilities reside at the third nucleotide codon position. In some embodiments, every codon, including the STOP codon, either has a G or a C in the third nucleotide codon position already or can be modified to have a G or a C at the third nucleotide codon position without changing the amino acid assignment. Thus, for any given nucleotide sequence, it is possible to have up to 100% G or C at each third nucleotide codon position (GC3) throughout the nucleotide sequence. Provided herein in an embodiment is a nucleotide sequence for RSV-F (set forth in SEQ ID NO: 17) that has been modified by changing one or more nucleotide bases within one or more codons whereby the RSV-F amino acid sequence is identical to the amino acid sequence encoded by the unmodified nucleotide sequence (set forth in SEQ ID NO: 2), and the overall combined percent of guanines and cytosines at the third nucleotide codon position is increased in the modified nucleotide sequence (100% GC3) compared to the unmodified nucleotide sequence (31% GC3; set forth in SEQ ID NO: 1). Also provided herein in an embodiment is a nucleotide sequence for sRSV-F (set forth in SEQ ID NOs: 4, 5 and 6) that has been modified by changing one or more nucleotide bases within one or more codons whereby the sRSV-F amino acid sequence is identical to the amino acid sequence encoded by the unmodified nucleotide sequence (set forth in SEQ ID NO: 7), and the overall combined percent of guanines and cytosines at the third nucleotide codon position is increased in the modified nucleotide sequences (58% GC3 for SEQ ID NO: 4; 76% GC3 for SEQ ID NO: 6; 100% GC3 for SEQ ID NO: 5) compared to the unmodified nucleotide sequence (31% GC3; set forth in SEQ ID NO: 3). Also provided herein in an embodiment is a nucleotide sequence for hPIV3-F (set forth in SEQ ID NO: 11) that has been modified by changing one or more nucleotide bases within one or more codons whereby the hPIV3-F amino acid sequence is identical to the amino acid sequence encoded by the unmodified nucleotide sequence (set forth in SEQ ID NO: 12), and the overall combined percent of guanines and cytosines at the third nucleotide codon position is increased in the modified nucleotide sequence (100% GC3) compared to the unmodified nucleotide sequence (29% GC3; set forth in SEQ ID NO: 10).

### Cis-Regulatory Elements

A nucleotide sequence can include a cis-regulatory element in certain embodiments. A cis-regulatory element or cis-element is a region of DNA or RNA that regulates the expression of genes located on that same molecule of DNA. Cis-regulatory elements can be binding sites for one or more trans-acting factors. A cis-element may be located 5′ to the coding sequence of the gene it controls (in the promoter region or further upstream), in an intron, or 3′ to an ORF, in the untranslated or untranscribed region, for example. Cis-regulatory elements can be added to engineered expression constructs to regulate mRNA transcription and/or mRNA processing in an in vitro or in vivo expression system. Examples of cis-regulatory elements include, without limitation, posttranscriptional regulatory elements (PRE), frameshift element, iron response element (IRE), internal ribosome entry site (IRES), TATA box, Pribnow box, SOS box, CAAT box, CCAAT box, Upstream Activation Sequence (UAS), Polyadenylation signals, AU-rich elements, and other cis-regulatory RNA elements known in the art.

A nucleotide sequences can include a posttranscriptional regulatory element in some embodiments. Posttranscriptional regulatory elements are cis-acting RNA elements that can increase cytoplasmic mRNA levels. Viruses often utilize posttranscriptional regulatory elements (PREs) to enhance gene expression through enabling mRNA stability and 3′ end formation, and to facilitate the export of mRNAs from the host cell nucleus to the cytoplasm. PREs also can be added to engineered expression constructs to help boost mRNA levels derived from transcription in an in vitro or in vivo expression system.

Examples of posttranscriptional regulatory elements include, without limitation, the posttranscriptional regulatory elements (PREs) of Hepatitis B virus (HPRE) and Woodchuck Hepatitis virus (WPRE). Both PREs are hepadnaviral cis-acting RNA elements that can increase the accumulation of cytoplasmic mRNA of an intronless gene by promoting mRNA exportation from the nucleus to the cytoplasm, enhancing 3′ end processing and stability. Both HPRE and WPRE can be used to increase expression efficiency from an expression vector. In some embodiments an HPRE is added to an expression construct. A WPRE sometimes is added to the expression construct. A WPRE, for example, can be placed in functional association with any of the nucleotide sequences provided herein (e.g., at a position 5′ to a nucleotide sequence, at a position 3′ to a nucleotide sequence, or at any position within an expression vector). A WPRE in some embodiments can be placed in functional association with any of the nucleotide sequences provided herein at a position downstream of the 3′ end of the nucleotide sequence. A WPRE in certain embodiments can be placed in functional position with any of the nucleotide sequences provided herein at a position downstream of the 3′ end of the nucleotide sequence and upstream of the 5′ end of a SV40 polyA region present in an expression vector.

### Cells

An expression vector can be propagated in any suitable cell line for protein expression. In some embodiments, an expression vector can be utilized in an adherent or non-adherent mammalian cell line that provides useful levels of protein expression from transfected expression vectors. The term “useful levels of protein expression” as used herein, refers to the production of a desired protein at levels that allow isolation of sufficient quantities of proteins for use in the production of protein. Non-limiting examples of mammalian cells include CHO cells, CHO-S cells, CAT-S cells, Vero cells, BSR-T7 cells, Hep-2 cells, MBCK cells, MDCK cells, MRC-5 cells, HeLa cells and LLC-MK2 cells.

Adherent Cells

Adherent cells generally require a surface, such as tissue culture plastic or microcarrier, which may be coated with extracellular matrix components to increase adhesion properties and provide other signals needed for growth and differentiation. Many cells derived from solid tissues are adherent. Another type of adherent culture is referred to as an organotypic culture. Organotypic culture methods typically involve growing cells in a three-dimensional (e.g., 3D) environment as opposed to two-dimensional (e.g., 2D) culture dishes. A 3D culture system is biochemically and physiologically more similar to in vivo tissue, but is more technically challenging to maintain. Non-limiting examples of adherent cell lines include Vero cells, MRC-5 cells and BSR-T7 cells.

Vero Cells

Vero cells are a cell line that has been widely used to propagate viruses to make vaccines. Vero cells were derived from kidney epithelial cells of the African Green Monkey (Cercopithecus aethiops) in 1962. Vero cells are susceptible to a broad range of viruses and often are used to develop vaccines against diseases associated with those viruses. Vero cells are used for many purposes, non-limiting examples of which include; (i) screening for the toxin of Escherichia coli (e.g., E. coli toxin was first named “Vero toxin” after this cell line, and later called “Shiga-like toxin” due to its similarity to Shiga toxin isolated from Shigella dysenteriae), (ii) as host cells for growing virus (e.g., to measure replication in the presence or absence of a research pharmaceutical, to test for the presence of rabies virus, or growth of viral stocks for research purposes), and (iii) as host cells for eukaryotic parasites (e.g., Trypanosomatids).

The Vero cell lineage is continuous and aneuploid. A continuous cell lineage can be replicated through many cycles of division and not become senescent. Aneuploidy is the characteristic of having an abnormal number of chromosomes. Vero cells also are interferon-deficient, unlike normal mammalian cells, Vero do not secrete type 1 interferons when infected by viruses, however, Vero cells have the interferon-alpha/beta receptor so they respond normally when interferon from another source is added to the culture.

Vero cells have been shown to have distinct lineages. Non-limiting examples of Vero “derived” cell lineages include; Vero cells (e.g., ATCC No. CCL-81), Vero 76 (e.g., ATCC No. CRL-1587, isolated from Vero cells in 1968), and Vero E6 cells (e.g., ATCC No. CRL-1586, cloned from Vero 76 cells). Vero cells have been adapted for growth in serum-free media.

MRC-5 Cells

MRC-5 cells (e.g., ATCC No. CCL-171) are a human, male diploid (e.g., 46 chromosomes, XY) cell line derived from normal fetal lung tissue. MRC-5 cells generally have fibroblast-like cell morphology. The cell lineage was first established in 1966. MRC-5 cells generally are not considered to be continuous (e.g., immortal), as senescence occurs after about 42-48 doublings, with as many as 60-70 doublings seen before senescence occurs. MRC-5 is susceptible to a wide range of human viruses, making it a useful cell line for viral propagation and vaccine production. Non-limiting examples of viruses to which MRC-5 cells are susceptible include Adenoviruses; Coxsackie A; Cytomegalovirus; Echovirus; Herpes simplex Virus; Poliovirus; Rhinovirus; Respiratory Syncytial Virus; and Varicella Zoster Virus. MRC-5 cells sometimes also are used for in vitro cytotoxicity testing.

BSR-T7/5 Cells

BSR-T7/5 cells are a BHK-21 (C-13) derived cell line constitutively expressing a T7 RNA polymerase. BSR-T7/5 cells have been used to establish animal free virus recovery systems for; the virus causing Newcastle disease, bovine respiratory syncytial, ebola virus, rabies viruses, rift valley fever virus and others. BSR-T7/5 cells also have been used to establish a vaccinia virus free vesicular stomatitis virus (VSV) recovery system.

BHK-21 (C-13) (e.g., ATCC No. CCL-10) is an immortalized baby Syrian golden hamster (e.g., Mesocricetus auratus) kidney cell line, which causes tumors in hamsters and nude mice. The parent line of BHK-21(C-13) was derived from the kidneys of five unsexed, 1-day-old hamsters in 1961. Following 84 days of continuous cultivation, interrupted only by an 8-day preservation by freezing, clone 13 (e.g., (C-13)) was initiated by single-cell isolation. BHK-21 derived cells are pseudodiploid with tetraploidy occurring at about 4%. BHK-21 derived cells have a fibroblast-like cell morphology. BHK-21 derived cells are susceptible to a number of viruses, non-limiting examples of which include; human adenovirus 25, reovirus 3, vesicular stomatitis virus and human poliovirus 2.

Non-Adherent Cells

Non-adherent cells normally exist in suspension without being attached to a surface. A non-limiting example of a non-adherent cell is a blood cell which normally exists circulating in the bloodstream, unlike the cells of tissues which generally are attached to each other and/or an underlying matrix. Certain cell lines have been modified for culturing in suspension cultures, allowing growth of the cells to a higher density than adherent conditions normally would allow. Non-limiting examples of non-adherent mammalian cells are CHO-derived cells, CHO-S cells and CAT-S cells. In some embodiments, protein expression vectors are propagated in CAT-S cells.

CHO and CHO-Derived Cells

CHO cells (e.g., CHO-K1, ATCC No. CCL-61, ECACC accession number 85051005) are an adherent cell line derived from Chinese hamster (e.g., Cricetulus griseus) ovary. The cell line was derived as a subclone from the parental CHO cell line initiated from a biopsy of an ovary of an adult Chinese hamster in 1957. The cells are proline auxotrophs and do not express the Epidermal growth factor receptor (EGFR). CHO cells have become a widely used cell line because of their rapid growth and high protein production. CHO derived cell lines frequently are used in research and biotechnology, especially when long-term, stable gene expression and high yields of proteins are required.

Non-adherent and loosely adherent CHO cell lineages have been isolated. Non-limiting examples of non-adherent CHO-derived cell lines include CHO-Pro5 (e.g., ATCC No. CRL-1781) and CHO-AA8 (e.g., ATCC No. CRL-1859). Additional CHO-derived lineages have been established from CHO-AA8, many, if not all, of which can be grown in suspension (e.g., CHO-UV41, ATCC No. CRL-1860; CHO-EM9, ATCC No. CRL-1861; CHO-UV20, ATCC No. CRL-1862; CHO-UV5, ATCC No. CRL-1865; CHO-UV24, ATCC No. CRL-1866; and CHO-UV135, ATCC No. CRL-1867) Many CHO-derived cells have an epithelial cell-like morphology, while some exhibit of fibroblast-like cell morphology.

CHO-S Cells

CHO-S cells (Invitrogen) are a stable, aneuploid, clonal isolate, derived from CHO-K1 cells. The CHO-S parental cell line was selected for growth and transfection efficiency. The CHO-S cells are adapted to serum-free suspension culture in CD CHO Medium (Invitrogen) supplemented with L-glutamine and HT Supplement for transient or stable expression of recombinant proteins.

CAT-S Cells

CAT-S cells are a CHO-K1-derived cell line that grow in suspension and show improved yields of proteins in comparisons performed against CHO-S cells. CAT-S cells (ECACC accession number 10090201) also have been adapted for serum free suspension growth.

### Transfection and Transformation

Transfection is a non-viral process of introducing nucleic acid or biomolecules into a cell. The term generally is used to refer to the introduction of nucleic acid or other biomolecules (e.g., proteins, antibodies, siRNA, RNA, oligonucleotides) into an animal-derived eukaryotic cell, whereas the term transformation is used to refer to bacterial cells and non-animal eukaryotic cells. Introduction of nucleic acid or other biomolecules into eukaryotic cells (e.g., mammalian cells) frequently involves providing a route or method for the nucleic acid to pass the cell membrane and enter the cell. Any suitable method for introducing nucleic acid into a host cell may be used. Non-limiting examples of methods suitable for use in transfection methods include electroporation, sonoporation (e.g., sound wave induced cell membrane holes), impalefection (e.g., introduction of DNA coated nanofiber into a cell), lipofection (e.g., fusing liposomes containing a nucleic acid with a host cell), calcium phosphate precipitation, cationic polymer mediated endocytosis (e.g., DEAE-dextran, or polyethylenimine transfection), biolistic or bio-particle transfection (e.g., DNA coated nanoparticles shot directly into the nuclei of a cell), optical transfection (e.g., use of a laser to produce a transient micropore in the cell membrane), heat shock, magnetofection, dendrimer transfection (e.g., highly branched organic compounds that bind DNA and can be transported across cell membranes), and the use of proprietary transfection reagents, protocols, and/or apparatus such as Lipofectamine (Invitrogen), Lipofectin, Dojindo Hilymax, Fugene (Fugent, LLC), jetPEI, Effectene, Nucleofector, Promofectin, Uptifectin, GENEporter or DreamFect.

Stable and Transient Transfection

Transfection sometimes is stable transfection and sometimes is transient transfection. Nucleic acid (e.g., DNA, RNA, PNA, the like or combinations thereof) introduced in the transfection process usually is not inserted into the nuclear genome, thus the introduced genetic material is eventually lost, typically around the time the cells undergo mitosis. A process in which transfected nucleic acid does not enter the nuclear genome and/or does not provide a selective advantage (e.g., selection against a toxin, G418 using the neomycin gene, for example) often is referred to as a transient transfection. For many applications (e.g., transient expression analysis, production of proteins, viruses, or antibodies in bioreactors for defined periods of growth) transient expression of a transfected gene is sufficient.

Stable transfection can be employed (e.g., integration into the genome, epigenetic element maintained through selection of a selectable marker) if it is desired that the transfected nucleic acid (e.g., DNA, RNA) remain in the genome of the cell and its daughter cells for an extended period of time. Stable transfectants with genomic integration can be further selected by removing the selection pressure for a defined period of time, followed by reapplying the selection pressure. Epigenetic elements often are lost when the selection pressure is removed for a period of time, thus reapplying the selection pressure can select against those cells that carried and lost an epigenetic resistance element.

Cytoplasmic and Nuclear Transfection

Transfection can be cytoplasmic transfection in some embodiments, or nuclear transfection in certain embodiments. Cytoplasmic transfection sometimes can lead to stable transfection, if the transfected material provides a selective advantage, and a selection pressure is maintained. Nuclear transfection, while less efficient, provides a higher incidence of stable integration of the transfected nucleic acid (e.g., DNA).

Many transfection methods insert the nucleic acid or other biomolecules in the cytoplasm of the host cell (e.g., liposome mediated transfection or lipofection, calcium phosphate precipitation, etc). Cells normally utilize a transport mechanism to carry mRNA from the cytoplasm to the nucleus (e.g., heterogeneous nuclear ribonucleoprotein-Al (hnRNP-A1)). The use of specific nuclear targeting signals in expression constructs or in other nucleic acids (e.g., mRNAs, siRNAs, plasmid DNA, linear DNA, etc) may increase the proportion of transfected material that is transported to the nucleus, and or may increase the proportion of cytoplasmically transcribed RNAs transported into the nucleus. A non-limiting example of a nuclear targeting signal is the M9 component of hnRNP-A1. Non-specific nuclear carriers also can be used to increase nuclear transfection efficiency. Non-limiting examples of non-specific nuclear carriers include; protamine, poly-lysine/pDNA, and cationic cholesterol.

Cellular DNA Integration

After nucleic acid has been transfected into a host cell and is transported to the nucleus, stable integration into the genome of the host cell (e.g., integration into a chromosome) can occur by a genetic recombination event. Genetic recombination is a process by which a molecule of nucleic acid (e.g., DNA, RNA) is broken and then joined to a different nucleic acid. Recombination can occur between molecules of DNA with similar nucleic acid sequences, as in homologous recombination, or between molecules of DNA with dissimilar nucleic acid sequences, as in non-homologous end joining. Recombination is a common method of DNA repair in both bacteria and eukaryotes. In eukaryotes, recombination also occurs in meiosis, where chromosomal crossover is facilitated. The term “chromosomal crossover” as used herein, refers to recombination between paired chromosomes, generally occurring during meiosis. During prophase I the four available chromatids are in tight formation with one another. While in this formation, homologous sites (e.g., sites with similar nucleic acid sequences) on two chromatids can mesh with one another, facilitating the exchange of genetic information (e.g., recombination). Non-homologous recombination can occur between DNA sequences that are not similar in sequence (e.g., have no sequence homology), and is often referred to as non-homologous end joining. Non-homologous end joining (NHEJ) is commonly used to repair double-strand breaks in DNA. NHEJ is referred to as “non-homologous” because the break ends are directly ligated without the need for a homologous template, in contrast to homologous recombination, which requires a homologous sequence to guide repair or crossover migration. Inappropriate NHEJ can lead to translocations and telomere fusion, hallmarks of tumor cells.

Homologous recombination signals can be engineered into a nucleic acid reagent or expression vector to further increase the chances of genomic integration when a transfected nucleic acid is transported to the nucleus. In some embodiments, a nucleic acid vector includes one or more recombinase insertion sites. A recombinase insertion site is a recognition sequence on a nucleic acid molecule that participates in an integration/recombination reaction by recombination proteins, as described above. A nucleic acid vector used for transfection of mammalian cells also can include topoisomerase integration sites for recombination, also as described above.

### Transcription

The terms “transcription”, “transcription activity”, “cytoplasmic transcription” and “nuclear transcription” as described herein refer to the process of generating a nucleic acid copy of a starting nucleic acid. Transcription often involves generating an RNA copy of a DNA nucleic acid, and sometimes also can involve generating a DNA copy of a starting RNA nucleic acid (e.g., reverse transcription).

Transcription can be characterized by the following steps for eukaryotic cells: (1) DNA unwinds/“unzips” as hydrogen bonds break; (2) free RNA nucleotides pair with complementary DNA bases; (3) RNA sugar-phosphate backbone forms, aided by RNA Polymerase; (4) hydrogen bonds of the uncoiled RNA/DNA hybrid break, freeing the newly transcribed RNA; and (5) the RNA is further processed and then moves through the small nuclear pores to the cytoplasm. Transcription also can be viewed as having five (5) stages, which overlap the steps described above. These five stages of transcription include pre-initiation, initiation, promoter clearance, elongation and termination.

Pre-Initiation

In eukaryotes, RNA polymerase recognizes a core promoter sequence in the DNA. Promoters are regions of DNA that promote transcription and in eukaryotes, often are found at −30, −75 and −90 base pairs upstream from the start site of transcription. It has been shown that mutations in core promoter sequences can abolish transcription initiation. RNA polymerase is able to bind to core promoters in the presence of various specific transcription factors. A non-limiting example of a core promoter sequence in eukaryotes is a short DNA sequence known as the TATA box, found 25-30 base pairs upstream from the start site of transcription. The TATA box, as a core promoter, is the binding site for a transcription factor known as TATA binding protein (TBP), which is itself a subunit of another transcription factor, called Transcription Factor II D (TFIID). After TFIID binds to the TATA box via the TBP, five more transcription factors and RNA polymerase combine around the TATA box in a series of stages to form a pre-initiation complex. One transcription factor, DNA helicase, has helicase activity is involved in the separating (e.g., unwinding of step (1) above) of opposing strands of double-stranded DNA to provide access to a single-stranded DNA template. Only a low, or basal, rate of transcription is driven by the pre-initiation complex alone. Other proteins known as activators and repressors, along with any associated coactivators or co-repressors, are responsible for modulating transcription rate.

Initiation

After the pre-initiation complex is formed, and free RNA's pair with complementary DNA bases, the first bond is synthesized, and the RNA polymerase translocates to allow bond formation at the next base.

Promoter Clearance

After the first bond is synthesized, DNA polymerase clears the promoter by translocating along the DNA. During this time the RNA polymerase sometimes releases the RNA transcript and produces truncated transcripts. This is called abortive initiation and is common for both eukaryotes and prokaryotes. Abortive initiation continues to occur until the sigma factor rearranges, resulting in the transcription elongation complex (e.g., which gives a 35 bp moving footprint). The sigma factor typically is released before 80 nucleotides of mRNA are synthesized. Once the transcript reaches approximately 23 nucleotides, the polymerase complex no longer slips and elongation can occur. Many of the steps in transcription are energy-dependent, consuming a molecule of adenosine triphosphate (ATP) for each bond formed. Promoter clearance coincides with phosphorylation of serine 5 on the carboxy terminal domain of RNA Pol in eukaryotes, which is in turn phosphorylated by TFIIH.

Elongation

As transcription proceeds, RNA polymerase traverses the template strand and uses base pairing complementarity with the DNA template to create an RNA copy. Although RNA polymerase traverses the template strand from 3′ to 5′, the coding (non-template) strand and newly-formed RNA can also be used as reference points, so transcription can be described as occurring 5′ to 3′. This produces an RNA molecule from 5′ to 3′, which is an exact copy of the coding strand, with the exception of substitution of uracil for thymine. mRNA transcription can involve multiple RNA polymerases on a single DNA template and multiple rounds of transcription (amplification of particular mRNA), so many mRNA molecules can be rapidly produced from a single copy (e.g., a single mRNA molecule) of a gene. Elongation also involves a proofreading mechanism that can replace incorrectly incorporated bases. In eukaryotes, the proofreading function may correspond with short pauses during transcription that allow appropriate RNA editing factors to bind. These pauses may be intrinsic to the RNA polymerase or due to chromatin structure.

Termination

Transcription termination in eukaryotes is believed to involve cleavage of the new RNA transcript, followed by template-independent addition of A nucleotides at newly generated 3′ end, in a process called polyadenylation. A putative eukaryotic termination signal that occurs upstream of the polyadenylation site is the nucleotide sequence AAUAAA. Transcription termination in bacteria involves specific termination proteins and/or terminator sequences.

Reverse Transcription

Certain viruses have the ability to transcribe RNA into DNA. RNA viruses have an RNA genome that is duplicated into DNA. The resulting DNA can be merged with the genomic DNA of the host cell. An enzyme involved in synthesis of DNA from an RNA template is called reverse transcriptase.

Cytoplasmic and Nuclear Transcription

Transcription generally occurs in the nucleus of a cell, the unspliced heteronuclear RNA is transported to the cytoplasm for splicing (e.g., removal of intron sequences) and the mature mRNA is transported to nucleus or other organelle (e.g., endoplasmic reticulum) for translation into protein. Replication, transcription and translation of some DNA viruses (e.g., Poxvirus) and many RNA viruses occurs in the cytoplasm of a cell. Cytoplasmic viral transcription sometimes involves the activity of viral genome encoded proteins.

Measuring and Detecting Transcription

Transcription can be measured and detected in a variety of ways. Non-limiting examples of assays that can be used to detect transcription include: nuclear run-on assay (e.g., measures the relative abundance of newly formed transcripts); RNase protection assay and ChIP-Chip of RNAP (e.g., detect active transcription sites); RT-PCR (e.g., measures the absolute abundance of total or nuclear RNA levels); DNA microarrays (e.g., measures the relative abundance of the global total or nuclear RNA levels); in situ hybridization (e.g., detects the presence of one or more specific transcripts); MS2 tagging (e.g., detection of MS2 RNA stem loop tags using a fusion of GFP and the MS2 coat protein, which has a high affinity, sequence specific interaction with the MS2 stem loops; the recruitment of GFP to the site of transcription is visualized as a single fluorescent spot); Northern blot (e.g., nucleic acid hybridization based probing of RNA blots with DNA or RNA complementary probes); RNA-Seq (e.g., sequencing techniques involving sequence analysis of whole transcriptomes (e.g., gene signature analysis), which allows the measurement of relative abundance of RNA; and detection of additional variations such as fusion genes, post-translational edits and novel splice sites).

### Cell Culture and Protein Production

The present technology provides serum-free cell culture medium and highly reproducible efficient scalable processes. In certain embodiments, provided are methods for producing relatively large quantities of protein in bioreactors, including without limitation single use bioreactors, and standard reusable bioreactors (e.g., stainless steel and glass vessel bioreactors).

In some embodiments, the present technology provides an enriched serum-free cell culture medium that supports proliferation of cells (e.g., CAT-S cells) to a high cell density. A cell culture medium used to proliferate cells can impact one or more cell characteristics including but not limited to, being non-tumorigenic, being non-oncogenic, growing as adherent cells, growing as non-adherent cells, having an epithelial-like morphology, supporting the replication of various viruses when cultured, and supporting the production of a desired protein product (e.g., antibody, virus particle and the like) as described herein. The use of serum or animal extracts in tissue culture applications for the production of protein is minimized, or eliminated to reduce the risk of contamination by adventitious agents (e.g., mycoplasma, viruses, and prions), to ensure pre-GMP conditions are met and to eliminate introduction of additional proteins which could complicate the protein purification process. Minimizing the number of manipulations required during cell culture procedures can significantly decrease the chances of contamination.

In some embodiments, cells are cultured in a serum-free culture medium (also referred to herein as “serum-free medium”). The serum-free medium sometimes is enriched, and sometimes cells are cultured in batch culture methods that do not utilize medium exchange or supplementation.

In certain embodiments, serum-free media comprises all the components of CD-CHO medium (Invitrogen).

In some embodiments, serum-free medium comprises a plant hydrolysate. Plant hydrolysates include but are not limited to, hydrolysates from one or more of the following: corn, cottonseed, pea, soy, malt, potato and wheat. Plant hydrolysates may be produced by enzymatic hydrolysis and generally contain a mix of peptides, free amino acids and growth factors. Plant hydrolysates are readily obtained from a number of commercial sources including, for example, Marcor Development, HyClone and Organo Technie. In certain embodiments, yeast hydrolysates my also be utilized instead of, or in combination with plant hydrolysates.

Yeast hydrolysates can be obtained from commercial sources including, for example, Sigma-Aldrich, USB Corp, Gibco/BRL and others. In certain embodiments, synthetic hydrolysates can be used in addition to, or in place of, plant or yeast hydrolysates. In some embodiments, serum-free medium comprises a plant hydrolysate at a final concentration of between about 0.1 g/L to about 5.0 g/L, or between about 0.5 g/L to about 4.5 g/L, or between about 1.0 g/L to about 4.0 g/L, or between about 1.5 g/L to about 3.5 g/L, or between about 2.0 g/L to about 3.0 g/L. In certain embodiments, serum-free medium comprises a plant hydrolysate at a final concentration of 2.5 g/L. In some embodiments, serum-free medium comprises a wheat hydrolysate at a final concentration of 2.5 g/L.

In certain embodiments, serum-free medium comprises a lipid supplement. Lipids that may be used to supplement culture medium include but are not limited to chemically defined animal and plant derived lipid supplements as well as synthetically derived lipids. Non-limiting examples of lipids which may be present in a lipid supplement include; cholesterol, saturated and/or unsaturated fatty acids (e.g., arachidonic, linoleic, linolenic, myristic, oleic, palmitic and stearic acids). Cholesterol may be present at concentrations between 0.10 mg/ml and 0.40 mg/ml in a 100× stock of lipid supplement. Fatty acids may be present in concentrations between 1 μg/ml and 20 μg/ml in a 100× stock of lipid supplement. Lipids suitable for medium formulations are readily obtained from a number of commercial sources including, for example HyClone, Gibco/BRL and Sigma-Aldrich.

In some embodiments, serum-free medium comprises a chemically defined lipid concentrate at a final concentration of between about 0.1× to about 2×, or between about 0.2× to about 1.8× or between about 0.3× to about 1.7×, or between about 0.4× to about 1.6×, or between about 0.5× to about 1.5×, or between about 0.6× to about 1.4×, or between about 0.7× to about 1.3×, or between about 0.8× and about 1.2×. In certain embodiments, serum-free medium comprises a chemically defined lipid concentrate (CDCL) solution at a final concentration of 1×, where X is between about 0.01 microgram/ml and about 40 microgram/mL in certain embodiments. In some embodiments, serum-free media comprises the chemically defined lipid concentrate (CDCL) solution at a final concentration of IX. In certain embodiments, serum-free media comprises trace elements. Trace elements which may be used include but are not limited to, CuSCVSH₂O, ZnSO4-7H₂O, Selenite-2Na, Ferric citrate, MnS(VH₂O, Na₂SiO₃-9H₂O, Molybdic acid-Ammonium salt, NH₄VO₃, NiSO₄-6H₂O, SnCl₂ (anhydrous), A1C13-6H₂O, AgNO₃, Ba(C₂H₃O₂)₂, KBr, CdCl₂, CoCl₂-6H₂O, CrCl₃ (anhydrous), NaF, GeO₂, KI, RbCl, ZrOCl₂-8H₂O. Concentrated stock solutions of trace elements are readily obtained from a number of commercial sources including, for example Cell Grow (see Catalog Nos. 99-182, 99-175 and 99-176).

In certain embodiments, serum-free media comprises one or more hormone, growth factor and/or other biological molecules. Hormones include, but are not limited to triiodothyronine, insulin and hydrocortisone. Growth factors include but are not limited to Epidermal Growth Factor (EGF), Insulin Growth Factor (IGF), Transforming Growth Factor (TGF) and Fibroblast Growth Factor (FGF). In some embodiments, serum-free media comprises Epidermal Growth Factor (EGF). Other biological molecules, include cytokines (e.g., Granulocyte-macrophage colony-stimulating factor (GM-CSF), interferons, interleukins, TNFs), chemokines (e.g., Rantes, eotaxins, macrophage inflammatory proteins (MIPs)) and prostaglandins (e.g., prostaglandins El and E2). In some embodiments, serum-free media comprises a growth factor at a final concentration of between about 0.0001 to about 0.05 mg/L, or between about 0.0005 to about 0.025 mg/L, or between about 0.001 to about 0.01 mg/L, or between about 0.002 to about 0.008 mg/L, or between about 0.003 mg/L to about 0.006 mg/L. In certain embodiments, serum-free media comprises EGF at a final concentration of 0.005 mg/L. In some embodiments, serum-free media comprises triiodothyronine at a final concentration of between about 1×10⁻¹² M to about 10×1010⁻¹² M, or between about 2×1010⁻¹² M to about 9×10⁻¹² M, or between about 3×10⁻¹² M to about 7×1010⁻¹² M, or between about 4×10⁻¹² M to about 6×10⁻¹² M. In certain embodiments, serum-free media comprises triiodothyronine at a final concentration of 5×1010⁻¹² M. In one embodiment, serum-free media comprises insulin at a final concentration of between about 1 mg/L to about 10 mg/L, or between about 2.0 to about 8.0 mg/L, or between about 3 mg/L to about 6 mg/L. In some embodiments, serum-free media comprises insulin at a final concentration of 5 mg/L. In certain embodiments, serum-free media comprises a prostaglandin at a final concentration of between about 0.001 mg/L to about 0.05 mg/L, or between about 0.005 mg/L to about 0.045 mg/L, or between about 0.01 mg/L to about 0.04 mg/L, or between about 0.015 mg/L to about 0.035 mg/L, or between about 0.02 mg/L to about 0.03 mg/L. In some embodiments, serum-free media comprises a prostaglandin at a final concentration of 0.025 mg/L. In certain embodiments, serum-free media comprises prostaglandin E1 at a final concentration of 0.025 mg/L.

In some embodiments, serum-free medium are fortified with one or more medium components selected from the group consisting of putrescine, amino acids, vitamins, fatty acids, and nucleosides. In certain embodiments, serum-free medium of the technology are fortified with one or more medium components such that the concentration of the medium component is about 1 fold, or about 2 fold, or about 3 fold, or about 4 fold, or about 5 fold higher or more than is typically found in a medium routinely used for propagating cell, such as, for example, Dulbecco's Modified Eagle's Medium/Ham's F12 medium (DMEM/F12). In some embodiments, serum-free media are fortified with putrescine. In certain embodiments, serum-free medium are fortified with putrescine such that the concentration of putrescine is about 5 fold higher, or more, than is typically found in DMEM/F12.

Non-limiting examples of fatty acids which may be used to fortify the serum-free medium include, unsaturated fatty acid, including but not limited to, linoleic acid and α-linolenic acid (also referred to as essential fatty acids), myristoleic acid, palmitoleic acid, oleic acid, arachidonic acid, eicosapentaenoic acid, erucic acid, docosahexaenoic acid; saturated fatty acids, including but not limited to, butanoic acid, hexanoic acid, octanoic acid, decanoic acid, dodecanoic acid, tetradecanoic acid, hexadecanoic acid, octadecanoic acid, eicosanoic acid, docosanoic acid, tetracosanoic acid and sulfur containing fatty acids including lipoic acid. In certain embodiments, the serum-free media are fortified with extra fatty acids in addition to that provided by a lipid supplement as described supra. In a specific embodiment, serum-free media are fortified with linoleic acid and linolenic acid. In some embodiments, serum-free media are fortified with linoleic acid and linolenic acid such that the concentrations of linoleic acid and linolenic acid are about 5 fold higher, or more, than is typically found in DMEM/F12.

Non-limiting examples of amino acids that may be used to fortify the serum-free medium include the twenty standard amino acids (alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine) as well as cystine and non-standard amino acids. In certain embodiments, one or more amino acids which are not synthesized by CAT-S cells or other mammalian cells, commonly referred to as “essential amino acids”, are fortified. For example, eight amino acids are generally regarded as essential for humans: phenylalanine, valine, threonine, tryptophan, isoleucine, methionine, leucine, and lysine. In some embodiments, serum-free media are fortified with cystine and all the standard amino acids except glutamine (DMEM/F12 is often formulated without glutamine, which is added separately), such that the concentrations of cystine and the standard amino acids are about 5 fold higher, or more, than is typically found in DMEM/F12. In certain embodiments, serum-free media comprises glutamine at a concentration of between about 146 mg/L to about 1022 mg/L, or between about 292 mg/L to about 876 mg/L, or between about 438 mg/L to about 730 mg/L. In some embodiments, serum-free media comprises glutamine at a concentration of about 584 mg/mL.

Non-limiting examples of vitamins which may be used to fortify the serum-free medium include, ascorbic acid (vit A), d-biotin (vit B7 and vit H), D-calcium pantothenate, cholecalciferol (vit D3), choline chloride, cyanocobalamin (vit B 12), ergocalciferol (vit D2), folic acid (vit B9), menaquinone (vit K2), myo-inositol, niacinamide (vit B3), p-amino benzoic acid, pantothenic acid (vit B5), phylloquinone (vit Ki), pyridoxine (vit B6), retinol (vit A), riboflavin (vit B2), alpha-tocopherol (vit E) and thiamine (vit Bi). In a specific embodiment, a serum-free medium is fortified with d-biotin, D-calcium, pantothenate, choline chloride, cyanocobalamin, folic acid, myo-inositol, niacinamide, pyridoxine, riboflavin, and thiamine such that the concentrations of the indicated vitamins are about 5 fold higher, or more, than is typically found in DMEM/F12.

Non-limiting examples of nucleosides that may be used to fortify a serum-free medium include; without limitation cytidine, uridine, adenosine, guanosine, thymidine, inosine, and hypoxanthine. In some embodiments, serum-free media are fortified with hypoxanthine and thymidine such that the concentrations of hypoxanthine and thymidine are about 5 fold higher, or more, than is typically found in DMEM/F12.

Additional components may be added to a serum-free cell culture medium, non-limiting examples of which include sodium bicarbonate, a carbon source (e.g., glucose), and iron binding agents. In one embodiment, serum-free media comprises sodium bicarbonate at a final concentration of between about 1200 mg/L to about 7200 mg/L, or between about 2400 mg/L and about 6000 mg/mL, or about 3600 mg/mL and about 4800 mg/mL. In some embodiments, serum-free media comprises sodium bicarbonate at a final concentration of 4400 mg/mL. In certain embodiments, a serum-free medium comprises glucose as a carbon source. In some embodiments, a serum-free medium comprises glucose at a final concentration of between about 1 g/L to about 10 g/L, or about 2 g/L to about 10 g/L, or about 3 g/L to about 8 g/L, or about 4 g/L to about 6 g/L, or about 4.5 g/L to about 9 g/L. In certain embodiments, a serum-free medium comprises glucose at a final concentration of 4.5 g/L. In various embodiments, additional glucose may be added to a serum-free medium that is to be used for the proliferation of CAT-S cells to high density and subsequent production of large quantities of an expressed protein product. Accordingly, in certain embodiments, a serum-free medium comprises an additional 1-5 g/L of glucose for a final glucose concentration of between about 5.5 g/L to about 10 g/L.

Non-limiting examples of iron binding agents that may be utilized in a serum-free medium include proteins such as transferrin and chemical compounds such as tropolone (see, e.g., U.S. Pat. Nos. 5,045,454; 5,118,513; 6,593,140; and PCT publication number WO 01/16294). In some embodiments, serum-free media comprises tropolone (2-hydroxy-2,4,6-cyclohepatrien-I) and a source of iron (e.g., ferric ammonium citrate, ferric ammonium sulfate) instead of transferrin. In certain embodiments, tropolone or a tropolone derivative will be present in an excess molar concentration to the iron present in the medium at a molar ratio of between about 5 to 1 and about 1 to 1. In some embodiments, serum-free media comprises tropolone or a tropolone derivative in an excess molar concentration to the iron present in the medium at a molar ratio of about 5 to 1, or about 3 to 1, or about 2 to 1, or about 1.75 to 1, or about 1.5 to 1, or about 1.25 to 1. In certain embodiments, serum-free media comprises Tropolone at a final concentration of 0.25 mg/L and ferric ammonium citrate (FAC) at a final concentration of 0.20 mg/L.

The addition of components to a medium formulation sometimes can alter the osmolality of the resulting formulation. In certain embodiments, the amount of one or more components typically found in DMEM/F12 is reduced to maintain a desired osmolality. In one embodiment, the concentration of sodium chloride (NaCl) is reduced in the serum-free medium described herein. In some embodiments, the concentration of NaCl in a serum-free media is between about 10% to about 90%, or about 20% to about 80%, or about 30% to about 70%, or about 40% to about 60% of that typically found in DMEM/F12. In certain embodiments, the final concentration of NaCl in a serum-free media is 50% of that typically found in DMEM/F12. In some embodiments, the final the concentration of NaCl in a serum-free media is 3500 mg/L.

In certain embodiments, the number of animal-derived components present in serum-free media are minimized or even eliminated. For example, commercially available recombinant proteins such as insulin and transferrin derived from non-animal sources (e.g., Biological Industries Cat. No. 01-818-1, and Millipore Cat. No. 9701, respectively) may be utilized instead proteins derived from an animal source. In some embodiments, all animal derived components are replaced by non-animal-derived products with the exception of cholesterol which may be a component of a chemically defined lipid mixture. To minimize the risks typically associated with animal derived products cholesterol may be from the wool of sheep located in regions not associated with adventitious agents including, but not limited to prions, in some embodiments.

A viral fusion protein (e.g., soluble viral fusion protein) sometimes is produced in cultured CAT-S cells. CAT-S cells are adapted for proliferation and/or production of a desired protein product in enriched serum-free culture medium. In certain embodiments, a serum-free medium can be used to support the proliferation of CAT-S cells, where the cells remain viable after at least 20 passages, or after at least 30 passages, or after at least 40 passages, or after at least 50 passages, or after at least 60 passages, or after at least 70 passages, or after at least 80 passages, or after at least 90 passages, or after at least 100 passages in the serum-free medium. In some embodiments, the proliferation of CAT-S cells is supported to a high density by serum-free medium. In certain embodiments, serum-free medium supports the proliferation of CAT-S cells to a density of least 5×10⁵ cells/mL, at least 6×10⁵ cells/mL, at least 7×10⁵ cells/mL, at least 8×10⁵ cells/mL, at least 9×10⁵ cells/mL, at least 1×10⁶ cells/mL, at least 1.2×10⁶ cells/mL, at least 1.4×10⁶ cells/mL, at least 1.6×10⁶ cells/mL, at least 1.8×10⁶ cells/mL, at least 2.0×10⁶ cells/mL, at least 2.5×10⁶ cells/mL, at least 5×10⁶ cells/mL, at least 7.5×10⁶ cells/mL, or at least 1×10⁷.

### Protein Production

The present technology provides methods for producing large quantities of proteins from a cell line (e.g., CAT-S cell line) transfected with an expression vector encoding a viral fusion protein (e.g., soluble viral fusion protein). In some embodiments, cells are grown to a cell density of between about 5×10⁵ to about 1×10⁷ prior to inducing protein production. In certain embodiments, protein production (e.g., expression) is induced during logarithmic growth of transfected CAT-S cells. In some embodiments, protein expression from a transfected construct is constitutive.

Protein production (e.g., expression of the protein product from the transfected nucleic acid) can be carried out in bioreactors, in some embodiments. Recombinant protein can be produced in a bioreactor under aerobic or anaerobic conditions. Bioreactors are commonly cylindrical, ranging in size from liters to cubic meters, and often are made of stainless steel. A bioreactor also may refer to a device or system meant to grow cells or tissues in the context of cell culture. On the basis of mode of operation, a bioreactor may be classified as batch, fed batch or continuous (e.g., a continuous stirred-tank reactor model). An example of a continuous bioreactor is a chemostat.

Continuous and Batch Process Reactors

A reactor is called a continuous reactor when the feed and product streams are continuously being fed and withdrawn from the system. In some embodiments, a reactor can have a continuous or recirculating flow, but no continuous feeding of nutrient or product harvest and still be considered a batch reactor. A batch reactor may or may not have medium withdrawal during the process run. Batch processes often are widely used due to their simplicity and usefulness in industries like vaccine production. Batch and fedbatch processes are similar processes that inoculate cells at a lower viable cell density in a chosen medium. Cells are allowed to grow exponentially with little to no external manipulation until nutrients are somewhat depleted and cells are approaching stationary phase. A portion of the culture is removed and replaced with fresh medium. The removal and replacement process can be repeated several times.

Fedbatch production of recombinant proteins differs from batch fed culturing in that while cells are still growing and the medium begins to become depleted, a concentrated feed medium (typically between 10× and 20× concentration of basal medium) is added continuously or intermittently to supply additional nutrients and support higher final cell densities. To accommodate the addition of fresh medium, fedbatch cultures are generally started at a lower volume than a batch culture, with respect to a bioreactor of a given size. In some embodiments, a fedbatch culture initial volume will be between about 40% to about 60% of a batch fed culture for a given bioreactor size.

Continuous cultures often incorporate the use of many of the same elements as batch and fedbatch cultures. Continuous cultures often involve the use of similar media and initial cell growth is effected in a similar manner. Once cells reach a certain density, inflow and harvest flow are initiated and the cells reach a steady state density.

Under optimum conditions, microorganisms or cells are able to perform their desired function with a 100 percent rate of success in a bioreactor. The bioreactor's environmental conditions like gas (i.e., air, oxygen, nitrogen, carbon dioxide) flow rates, temperature, pH and dissolved oxygen levels, and agitation speed/circulation rate often are monitored and controlled to optimize protein production.

A non-limiting example of a bioreactor suitable for culturing cells to high densities for large scale protein production is the Wave Bioreactor System (GE Healthcare). Wave bioreactors combine aspects of single use bioreactors (e.g., disposable cell bag) and permanent bioreactors (rocking platform and monitoring system).

In some embodiments, a viral fusion protein (e.g. RSV-F or hPIV3-F) is expressed in a transfected cell line propagated in a bioreactor. In certain embodiments, the cells are harvested to isolate the desired protein product. In some embodiments, the desire protein product is secreted into the culture media by the transfected cell, and the protein is harvested from the culture medium. In some embodiments the desired protein product is produced at between about 0.001 grams per liter to about 40 grams per liter (e.g., about 0.001 grams/liter (g/L), 0.002 g/L, 0.006 g/L, 0.01 g/L, 0.05 g/L, 0.1 g/L, 0.2 g/L, 0.3 g/L, 0.4 g/L, 0.5 g/L, 0.6 g/L, 0.7 g/L, 0.8 g/L, 0.9 g/L, 1.0 g/L, 1.1 g/L, 1.2 g/L, 1.3 g/L, 1.4 g/L, 1.5 g/L, 1.6 g/L, 1.7 g/L, 1.8 g/L, 1.9 g/L, 2.0 g/L, 2.5 g/L, 3.0 g/L, 3.5 g/L, 4.0 g/L, 4.5 g/L, 5.0 g/L, 5.5 g/L, 6.0 g/L, 6.5 g/L, 7.0 g/L, 7.5 g/L, 8.0 g/L, 8.5 g/L, 9.0 g/L, 9.5 g/L, 10.0 g/L, 10.5 g/L, 11.0 g/L, 11.5 g/L, 12.0 g/L, 12.5 g/L, 13.0 g/L, 13.5 g/L, 14.0 g/L, 14.5 g/L, 15.0 g/L, 17.5 g/L, 20.0 g/L, 22.5 g/L, 25.0 g/L, 27.5 g/L, 30.0 g/L, 32.5 g/L, 35.0 g/L, 37.5 g/L, and 40.0 g/L).

### Protein Purification

Protein purification is a process that includes one or more steps intended to isolate a single type of protein from a complex mixture. Separation steps often exploit differences in protein size, physico-chemical properties and/or binding affinity. In some embodiments, separation is based on size, shape, charge, hydrophobicity, biological activity, the like or combinations thereof. In some embodiments, a protein can be purified using non-specific purification methods, affinity purification methods or combinations thereof. In certain embodiments, affinity purification methods can be based on specific tag recognition (e.g., 6×His tag) or based on a specific epitope (e.g., FLAG affinity purification).

Many forms of protein purification can be combined with various chromatography methods, including the use of various separation media and various types of chromatography (e.g., batch, column HPLC, FPLC, etc.). Protein purification materials and methods are known in the art, and each protein purification scheme is dependent on a number of factors including size, pl, hydrophobicity and the like. Therefore to isolate a protein to 90% or greater purity may require a combination of purification schemes that can require optimization for each protein. Non-limiting examples of types of chromatography that can be used to purify proteins includes, ion-exchange chromatography (e.g., separates compounds according to the nature and degree of their ionic charge), affinity chromatography (e.g., separation technique based upon molecular conformation, which frequently utilizes application specific resins. These resins have ligands attached to their surfaces which are specific for the compounds to be separated.), metal binding chromatography (e.g., a sequence of 6 to 8 histidines in the N- or C-terminal ends of a protein binds strongly to divalent metal ions such as nickel and cobalt. The protein can be passed through a column containing immobilized nickel ions, which binds the polyhistidine tag.), immunoaffinity chromatography (e.g., uses the specific binding of an antibody to the target protein to selectively purify the protein. The procedure involves immobilizing an antibody to a column material, which then selectively binds the protein, while everything else flows through.), high performance liquid chromatography (e.g., a form of chromatography applying high pressure to drive the solutes through the column faster, causing diffusion to be limited and resolution improved.), the like and/or combinations thereof. A commonly used form of HPLC is “reversed phase” HPLC, where the column material is hydrophobic. The proteins are eluted by a gradient of increasing amounts of an organic solvent, such as acetonitrile and proteins elute according to their hydrophobicity.

A recombinant viral fusion protein (e.g., soluble viral fusion protein) can be purified to a desired level of purity. In some embodiments, a viral fusion protein is purified to a purity of 90% or greater (e.g., about 91%, 92%, 93%, 94%, 95%, 96%, 97%, 98%, 99%, 99.5%, 99.9%, 99.50%, 99.95%).

### Protein Quantification

Protein identification and quantification can be carried out at any step of purification. In some embodiments, aliquots of protein sample are obtained at each step of purification for determination of relative fold purification and protein purity. In some embodiments, SDS-PAGE is used to monitor purification success, by identifying any reduction in the number of contaminating material (e.g., other proteins) in a sample after each successive step of purification. The use of protein standard curves on an SDS-PAGE gel also can facilitate determination of protein concentration.

A method for determining protein concentration is a Bradford protein assay. The latter can be determined by the Bradford total protein assay or by absorbance of light at 280 nm. Another method that can be used for protein quantification is Surface Plasmon Resonance (SPR). SPR can detect binding of label free molecules on the surface of a chip. Additional non-limiting protein quantification methods include, Lowry assay, Fluoroprofile protein quantification, and micro pyrogallol red protein quantification method. The amount of protein in a preparation can be expressed as a total amount of protein, concentration of protein or active concentration of the protein as the percent of the total protein, in some embodiments. The viral fusion proteins provided herein also can be quantified by immunoassay methods provided herein including, for example, quantitative sandwich ELISA, and other immunoassays including, for example, indirect ELISA, competitive ELISA, sensitive fluorescent enzyme immunoassay (FEIA), nano-immunoassay (NIA), radioimmunoassays, magnetic immunoassays and any assay known in the art for protein quantification.

### Detectable Features and Solid Supports

A nucleic acid or recombinant viral fusion protein (e.g., soluble viral fusion protein) may be modified with one or more detectable features in some embodiments. A nucleic acid may be modified by a detectable feature at the 5′ end, the 3′ end, in-between the 5′ and 3′ ends and combinations thereof. A protein may be modified by a detectable feature at the N-terminus, the C-terminus, in-between the N-terminus and C-terminus and combinations thereof. The detectable feature may be incorporated as part of the synthesis, or added prior to detecting, quantifying or using the nucleic acid or recombinant protein. Incorporation of a detectable feature may be performed in liquid phase or on solid phase in some embodiments.

Any detectable feature suitable for detection of a nucleic acid or recombinant protein can be appropriately selected. Examples of detectable features are fluorescent labels such as fluorescein, rhodamine, and others (e.g., Anantha, et al., Biochemistry (1998) 37:2709 2714; and Qu & Chaires, Methods Enzymol. (2000) 321:353 369); radioactive isotopes (e.g., ¹²⁵I, 131I, 35S, 31P, 32P, 33P, 14C, 3H, 7Be, 28Mg, 57Co, 65Zn, 67Cu, 68Ge, 82Sr, 83Rb, 95Tc, 96Tc, 103Pd, 109Cd, and 127Xe); light scattering labels (e.g., U.S. Pat. No. 6,214,560, and commercially available labels (Invitrogen); chemiluminescent labels and enzyme substrates (e.g., dioxetanes and acridinium esters), enzymic or protein labels (e.g., green fluorescence protein (GFP) or color variant thereof, luciferase, peroxidase); other chromogenic labels or dyes (e.g., cyanine), and other cofactors or biomolecules such as digoxigenin, strepdavidin, biotin (e.g., members of a binding pair such as biotin and avidin for example), affinity capture moieties, 3′ blocking agents (e.g., phosphate group, thiol group, phosphorothioate, amino modifier, biotin, biotin-TEG, cholesteryl-TEG, digoxigenin NHS ester, thiol modifier C3 S—S (Disulfide), inverted dT, C3 spacer) and the like. In some embodiments an oligonucleotide species composition may be labeled with an affinity capture moiety. Also included in detectable features are those labels useful for mass modification for detection with mass spectrometry (e.g., matrix-assisted laser desorption ionization (MALDI) mass spectrometry and electrospray (ES) mass spectrometry).

A nucleic acid or recombinant protein can be associated with a solid support. The term “solid support” or “solid phase” as used herein refers to an insoluble material with which nucleic acid or protein can be associated, and the terms can be used interchangeably. Examples of solid supports include, without limitation, silica gel, glass (e.g. controlled-pore glass (CPG)), nylon, Sephadex®, Sepharose®, cellulose, a metal surface (e.g. steel, gold, silver, aluminum, silicon and copper), a magnetic material, a plastic material (e.g., polyethylene, polypropylene, polyamide, polyester, polyvinylidenedifluoride (PVDF)), chips, flat surface filters, one or more capillaries and/or fibers, arrays, filters, beads, beads (e.g., paramagnetic beads, magnetic beads, microbeads, nanobeads) and particles (e.g., microparticles, nanoparticles). Beads and/or particles may be free or in connection with one another (e.g., sintered). In some embodiments, the solid phase can be a collection of particles. In certain embodiments, the particles can comprise silica, and the silica may comprise silica dioxide. In some embodiments the silica can be porous, and in certain embodiments the silica can be non-porous. In some embodiments, the particles further comprise an agent that confers a paramagnetic property to the particles. In certain embodiments, the agent comprises a metal, and in certain embodiments the agent is a metal oxide, (e.g., iron or iron oxides, where the iron oxide contains a mixture of Fe²⁺ and Fe³⁺).

### EXAMPLES

The examples set forth below illustrate certain embodiments and do not limit the technology.

### Example 1

### Materials and Methods

The materials and methods set forth in this Example were used to perform the experiments described in Examples 2-5, except where otherwise noted.

A. Full-Length RSV-F Expression

The following methods apply to experiments performed using full-length RSV-F expression constructs.

1. Cells and Viruses

BSR-T7, MRC-5, Vero, and HEp-2 cells were maintained in MEM containing 5-10% FBS and several of the following supplements: 2% tryptose phosphate broth (Sigma), 2-4 mM L-glutamine, 50 μg/ml gentamicin, 1 mM sodium pyruvate, 2 mM L-glutamine, nonessential amino acids, 50 U/ml penicillin, and 50 μg/ml streptomycin. 293F cells were maintained in 293FREESTYLE. 293Ad cells were maintained in DMEM supplemented with 10% FBS, 100 U/ml penicillin, and 100 μg/ml streptomycin. The FLP-IN TREX system available from Invitrogen was used to create stable, tetracycline inducible expression of F_{GC-WPRE} and F_{GC} in a 293-derived cell line. These cells were maintained in DMEM containing high glucose and 2 mM glutamate supplemented with 10% Tet-approved FBS (Clontech), 100 U/ml penicillin and 100 μg/ml streptomycin, 15 μg/ml blasticidin, and 150 μg/ml hygromycin. For induction of F protein expression, tetracycline (Teknova) was added to the medium for a final concentration of 15 μg/ml. Cell lines were maintained in 5% CO₂ at 37° C., except 293F cells which were maintained in 8% CO₂. Cell culture reagents were obtained from Invitrogen unless otherwise stated. RSV A Long strain was propagated in HEp-2 cells, and virus stocks were made by infecting a freshly prepared confluent monolayer of HEp-2 cells at an MOI of 0.2, harvesting virus at 48 h post-infection by scraping cells into supernatant, placing cells and supernatant into a tube, sonicating twice at 50% power, and pelleting the cell debris by centrifugation. An equal volume of 50% sucrose in water solution (w/v) was added to the virus harvests, aliquoted, flash frozen in a dry ice/ethanol bath, and stored at −80° C. RSV A Long infections for western blots were carried out at an MOI of 0.5 and harvested 24 h post infection. The recombinant b/hPIV3/RSV F2, a chimeric bovine/human parainfluenza virus expressing RSV F from the second gene position, was propagated as previously described (Tang et al., 2003 J. Virol. 77, 10819-10828).

2. F Protein Expression Vectors

The RSV-F gene sequences used in this study are from RSV subtype A and include the wild-type F gene sequence from the Long strain (F_{Long}) cloned directly from infected HEp-2 cells by RT-PCR, the wild-type F protein gene sequence from the A2 strain (F_{A2}) cloned in a similar manner, the A2 strain F gene sequence codon-optimized by DNA 2.0 Inc. using a Monte Carlo algorithm (Fₒₚₜ) (Villalobos et al., 2006 BMC Bioinform. 7, 285), and the A2 strain F gene sequence enriched for the percentage of G or C nucleotides in a manner that preserved the amino acid sequence was synthesized by DNA 2.0, Inc. (F_{GC}). The F_{Long} and F_{A2} sequences are 98% identical on both the nucleotide and amino acid levels. The F_{A2}, Fₒₚₜ, and F_{GC} sequences were first cloned into the pCMVscript expression vector (Stratagene). The F_{A2}, Fₒₚₜ, and F_{GC} sequences were also cloned into the b/hPIV3 recombinant virus vector, which was previously described (Tang et al., 2003 J. Virol. 77, 10819-10828). In addition, the F_{Long}, F_{A2}, Fₒₚₜ, and F_{GC} sequences were cloned into an expression vector, pEBNA, containing a CMV_{IE} promoter, an SV40 polyadenylation sequence, and the Epstein barr virus nuclear antigen (EBNA) sequence. The pEBNAF protein expression vectors include an optimal Kozak sequence, ggcggcacc, directly upstream of the F protein gene start codon. The WPRE sequence cloned from the System Biosciences plasmid pCDH1-MCS1-Ef1-Puro was placed after the F gene stop codon and before the SV40 polyadenylation sequence as illustrated in FIG. 1A. Sequences were confirmed by dideoxy sequencing analysis (Applied Biotechnology, Inc.). Plasmids were purified using Qiagen DNA purification kits for use in transfections.

3. Transfections

BSR-T7, MRC-5, and Vero cell lines were seeded in 6-well plates for 80% confluency at the time of transfection. For transfection, 1 μg of plasmid DNA and 4 μl of LIPOFECTAMINE2000 (Invitrogen) were used according to Invitrogen's protocol. The next day, the transfection medium was replaced by 2 ml of OPTI MEM+gentamycin and the cells were further incubated. In some instances, cells were cultured for up to 72 hours before lysates were collected and analyzed for protein expression via Western blot, as described below.

293F cells were transfected according to Invitrogen's protocol with 40 μl 293FECTIN (Invitrogen) at 1×10⁶ cells/ml in a 30-ml culture with the same molar amount of each expression vector, accounting for the larger size of the plasmids containing the WPRE, or about 30 μg. To account for uniformity of transfection of the 293F cells, 1.5 μg of the pMetLuc2-Control vector from the READY-TO-GLOW secreted Luciferase Reporter kit (Clontech) was cotransfected with the F protein encoding expression vectors and an assay for secreted luciferase was performed 24 h post-transfection according to Clontech's protocol. 293Ad cells were transfected at 1×10⁶ cells/well in 6-well plates with the same molar amounts of each expression vector or about 4 μg and 5 μl LIPOFECTAMINE2000.

4. Western blots

Protein lysates were harvested by removing the medium and adding Laemmli buffer with β-mercaptoethanol or an NP-40 containing lysis buffer followed by centrifugation of debris. Cell lysates were separated on a 12% Tris-Glycine READY GELS or 10% NU-PAGE gels (Invitrogen) and transferred to PVDF or nitrocellulose membrane (Invitrogen). The membrane was blocked in phosphate buffered saline (PBS) or tris buffered saline and 0.01% TWEEN20 (TBS-T) containing 5% nonfat dry milk. For F protein detection, a high-affinity anti-RSV F protein monoclonal antibody (motavizumab) (Wu et al., 2007 J. Mol. Bio. 368, 652-665) was used as the primary detection antibody. Washes were performed with PBS and 0.05% TWEEN20 (PBS-T) or TBS-T. HRP-conjugated rabbit or donkey anti-human antibody (DAKO or Jackson ImmunoResearch) was added to the blot in PBS-T or TBS-T as the secondary detection antibody. The blot was washed with PBS-T or TBS-T, developed with an ECL kit (Amersham Pharmacia) or LUMIGLO (KPL), and exposed to KODAK Film. For β-actin detection, the blotting procedure was the same except the primary antibody used was anti-β-actin monoclonal antibody (Millipore MAB1501) or anti-β-actin rabbit polyclonal antibody (Sigma) and the secondary antibody used was goat anti-mouse antibody (Molecular Probes) or goat anti-rabbit antibody (Sigma).

In some instances, cells were cultured up to 72 hours post-transfection before lysates were generated by the addition of 0.3 mL of Laemmli buffer containing betamercaptoethanol per well. Lysates were collected and run on 12% SDS polyacrylamide gels and proteins were transferred to PVDF membrane. Blots were blocked in 5% skim milk powder and then incubated with 1 μg/ml Motavizumab in PBS-TWEEN20 containing 1% skim milk powder (w/v). Following incubation with 1:5000 dilution of an anti-human HRP conjugated secondary antibody, protein bands were visualized with ECL substrate (GE Healthcare) and variable length of exposure to KODAK Biomax film. Blots were subsequently re-probed with anti-actin monoclonal antibody to allow for normalization of protein loading.

5. RT-PCR

RNA was purified from transfected 293F cells with the RNEASY RNA purification kit (Qiagen) according to Qiagen's protocol. The RNA was subjected to DNase treatment using amplification grade DNase (Invitrogen) according to Invitrogen's protocol with an additional ethanol precipitation step at the end of the protocol. The first strand synthesis was carried out with oligo(dT)N and Moloney murine leukemia virus (MMLV) reverse transcriptase (RT) (Invitrogen) according to Invitrogen's protocol. The second strand synthesis included a forward primer complementary to a region downstream to the transcriptional start site within the CMV promoter and a reverse primer complementary to the last 21 nucleotides of each F gene sequence including the stop codon. For the second strand synthesis, the thermal profile was as follows: 95° C. for 3 min, followed by 30 cycles of 95° C. for 30 s, 48° C. for 30 s, and 72° C. for 2 min, followed by a final extension step of 72° C. for 10 min.

6. 3′ Rapid Amplification of cDNA Ends (RACE) RT-PCR

RNA was purified from transfected 293F cells as described earlier. The 3′ RACE analysis was carried out with a 3′ RACE kit (Invitrogen) following Invitrogen's protocol for the non-nested second strand synthesis option. Forward gene specific primers not included in the kit for second strand synthesis are complementary to the first 21 nucleotides of each F gene sequence including the start codon. For the second strand synthesis, the thermal profile was identical to that described for RT-PCR.

7. TOPO TA Cloning and Sequencing

Major DNA species from the 3′ RACE RT-PCR reaction were extracted from agarose gel using a gel extraction kit (Qiagen). Four microliters of the gel-extracted PCR fragments were added to 1 μl of TOPO TA pCR2.1 vector (Invitrogen) along with 1 μl of the supplied buffer and incubated at room temperature for 30 min prior to transformation of TOP10 cells (Invitrogen) using Invitrogen's protocol. Plasmid DNA from bacteria colonies was isolated using a DNA mini-prep kit (Qiagen). Purified plasmids were subjected to dideoxy sequencing analysis using the M13 primer and the Big Dye terminator kit. The sequencing reactions were performed as described earlier.

8. Microscopy

Images were recorded using a NIKON TE2000-E microscope (Nikon Instruments Inc.) with a 109 NA 0.25 objective. The system was equipped with a COOL SNAP ES2 camera (Photometrics) driven by METAMORPH, version 7.1.3.0 (Molecular Devices). A NIKON B-2 E/C FITC filter with an excitation of 465-495 nm was used for visualizing GFP expression.

9. Determination of Antibody Bound to F Protein Expressing Cell Lines

Motavizumab and R3-47, a negative control monoclonal antibody, were Eu³⁺-labeled using the DELFIA Eu-N1-ITC labeling chelate and were characterized according to the manufacturer's directions (Perkin Elmer). Octet monoclonal antibody affinity assays (ForteBio) to purified RSV-F protein were run to confirm Eu³⁺ labeling did not alter the monoclonal antibody binding properties.

TREx-F_{GC-WPRE} and -F_{GC} cell lines were grown to confluence and F protein expression induced with tetracycline as described. 20 and 44 h post induction, cells were collected and resuspended to ˜1×10⁷ viable cells/ml in 50% media/50% LR Binding Buffer (Tris based buffer system, Perkin Elmer). Approximately 1×10⁵ cells were mixed with 25 nM Eu³⁺ labeled motavizumab or R3-47 in LR Binding Buffer in a 100-μl reaction volume. The cells plus monoclonal antibody were incubated for 1 h at 4° C., and then added to Pall GHP vacuum filter plates. Unbound monoclonal antibody was washed away with 5×200 μl washes with DELFIA Assay Wash Buffer (Perkin Elmer), and Eu³⁺ fluorescence was released with the addition of 200 μl Enhancement Solution (Perkin Elmer). Time Resolved Fluorescence was read on an Envision Reader (Perkin Elmer) after 1 h incubation at 37° C. with gentle shaking. Eu³⁺ counts were converted to bound ng IgG, and specific bound motavizumab calculated by subtracting R3-47 nonspecific binding from total bound motavizumab.

B. Soluble RSV-F and hPIV3-F Expression

The following methods apply to experiments performed using soluble RSV-F and/or hPIV3-F expression constructs.

1. Expression Construct Generation

a. Soluble RSV-F Expression Constructs

Several modified nucleotide sequences were evaluated for expression of soluble RSV-F protein (sRSV-F). The nucleotide sequences encode the identical sRSV-F amino acid sequence and were generated from the wild-type RSV-F subtype A2 strain cloned within the pCMVscript recombinant vector as previously described (Huang et al., 2010 Virus Genes 40: 212-221). The wild-type sequence of the RSV-F construct was amplified by PCR from strain A2. The nucleotides encoding the 50 amino acid C-terminal transmembrane domain of the RSV-F protein, corresponding to amino acid 525-574 of SEQ ID NO: 2, were deleted to allow for secretion of the protein into the cell culture medium. For the codon-optimized construct (OPT), codon-optimization of the wild-type sequence and gene synthesis was performed by DNA 2.0 (Menlo Park, Calif.). The high guanine/cytosine content (GC3) construct was also synthesized by DNA 2.0 and was generated by changing every 3rd base pair in the wild-type sequence to either guanine or cytosine. No change was made for codons in which the 3rd base pair was already guanine or cytosine. The medium guanine/cytosine content (HL2) construct was generated by DNA 2.0 the same way as the GC3 construct, although not all codons were changed in the HL2 construct. The GH5 synthetic RSV-F construct was designed to house Fsel and Sbfl restriction sites at the 5′ and 3′ ends of the coding region, respectively, and was generated by Integrated DNA Technologies (Coralville, Iowa). The PCR primers used for initial cloning into the pCMV vector are presented in Table 2 below.

TABLE 2
Primer sequences for RSV-F amplification and cloning into pCMV

| | 5′Primer Sequences | 3′Primer Sequences |
|---|---|---|
| Wild-type | 5′-gcatgagctcatggagttg | 5′-gcatctcgaggttactaaatg |
| construct | ctaatcctcaaagc-3′ | caatattatttataccac-3′ |
| | (SEQ ID NO: 18) | (SEQ ID NO: 19) |
| Codon optimized | 5′-gcatgagctcatggaacttctt | 5′-gcatctcgagctaatttgaaaa |
| construct (OPT) | attctcaaagccaatgcg-3′ | ggctatgttattgatcccagac-3′ |
| | (SEQ ID NO: 20) | (SEQ ID NO: 21) |
| GC-enriched | 5′-gcatgctagcatggagtt | 5′-gcataagcttttagttgctg |
| construct (GC3) | gctcatcctcaaggc-3′ | aacgcgatgttgttg-3′ |
| | (SEQ ID NO: 22) | (SEQ ID NO: 23) |

TABLE 3

| Soluble RSV-F | | % GC | % GC3 | |
|---|---|---|---|---|
| Construct | Synthesized by | Content | Content | SEQ ID NO |
| Wild-type | PCR | 35 | 31 | 3 |
| OPT | DNA2.0 | 46 | 58 | 4 |
| GC3 | DNA2.0 | 58 | 100 | 5 |
| HL2 | DNA2.0 | 51 | 76 | 6 |
| GH5 | Integrated DNA | 59 | 95 | — |

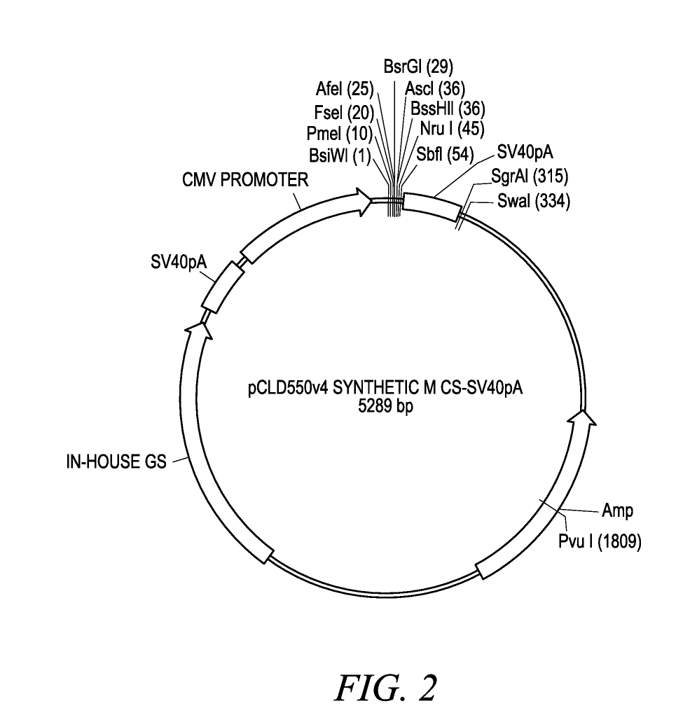

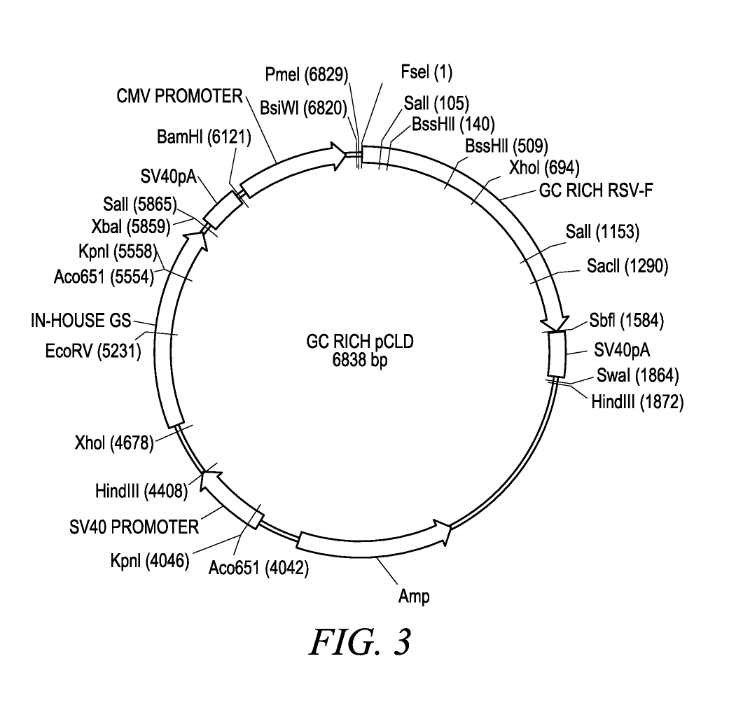

For expression of these soluble RSV-F constructs in CAT-S cells, each construct was subcloned into the pCLD550v4-synthetic MCS pA vector. Briefly, PCR reactions were performed using the RSV-F constructs described above which include wild-type soluble, codon-optimized (OPT), the GC-enriched construct (GC3), the medium GC construct (HL2) and GH5, all within the pCMVscript vector. PCR primers were designed to introduce Fsel and Sbfl restriction sites at the 5′ and 3′ ends of the amplified fragment to facilitate downstream subcloning (see Table 4 below). A stop codon was likewise introduced proximal to the transmembrane domain of each cDNA. This strategy was used to ensure the resulting proteins would be secreted from cells by terminating transcription prior to the transmembrane anchor present in the wild-type protein. 2 μl of each amplification reaction was ligated into the TOPO TA vector (Invitrogen) and clones screened by restriction digestion and sequence analysis. Representative sRSV-F clones harboring the proper sequence were cut from TOPO TA vector by digestion with Fsel & Sbfl and ligated into similarly cut pCLD550v4-synthetic MCS-pA vector (FIG. 2). The soluble high GC construct (GC3) cloned into the pCLD vector is illustrated in FIG. 3. Clones were screened by extensive restriction enzyme digestion and representative clones were confirmed by DNA sequencing.

TABLE 4
Primer sequences for sRSV-F amplification and cloning into pCLD

| | 5′Primer Sequences | 3′Primer Sequences |
|---|---|---|
| Wild-type soluble | 5′-GGC CGG CCA TGG AGT | 5′-CCT GCA GGT TAA TTT |
| construct | TGC TAA TCC TCA AAG C-3′ | GTG GTG GAT TTA CCG |
| | (SEQ ID NO: 24) | GC-3′ (SEQ ID NO: 25) |
| Codon optimized | 5′-GGC CGG CCA TGG AAC | 5′-CCT GCA GGT TAG TTT |
| construct (OPT) | TTC TTA TTC TCA AAG CC- | GTG GTG GAT TTA CCG |
| | 3′ (SEQ ID NO: 26) | GC-3′ (SEQ ID NO: 27) |
| GC-enriched | 5′-GGC CGG CCA TGG AGT | 5′-CCT GCA GGT TAG TTC |
| construct (GC3) | TGC TCA TCC TCA AGG CC- | GTG GTG GAC TTG CCG |
| | 3′ (SEQ ID NO: 28) | GCG-3′ (SEQ ID NO: 29) |

TABLE 4: The 5′ and 3′ primer sequences utilized for amplification and construction of the three sRSV-F constructs are presented here. Integrated within the 5′ primer sequences are Fsel restriction sites and within the 3′ primer sequences are Sbfl restriction sites (underlined). For each construct, a premature stop codon (TTA; indicated in bold) was inserted at a position proximal to the transmembrane domain to generate truncated forms of the protein.

For clonal cell line development, expression constructs were linearized prior to transfection by restriction enzyme digestions using either Sspl (GC-enriched sRSV-F in pCLD) or Pvul (wild-type soluble) and codon-optimized sRSV-F in pCLD). Animal component free Sspl and Pvul were obtained from Roche to ensure cGMP specifications were met. Linearized DNAs were phenol-chloroform purified and analyzed by gel electrophoresis and spectrophotometer to determine linearization and quantity respectively.

In some cases, each sequence-confirmed DNA plasmid was prepared by the Qiagen MaxiPrep procedure (Qiagen) and quantified via Nanodrop (Thermo-Fisher Scientific) analysis. Plasmids were subsequently linearized by restriction digest cleavage using Sapl enzyme (New England Biolabs) that has only one cleavage site near the Ampicillin resistance ORF and hence is far removed from both the fusion protein and glutamate synthetase coding regions. Linearized plasmid DNA was ethanol-precipitated, quantified by Nanodrop analysis, and subsequently used for transfection of suspension CAT-S and/or CHO-S cells.

b. Soluble hPIV3-F Expression Constructs

The wild-type sequence of hPIV3 fusion protein (hPIV3-F) was amplified by PCR using a template for wild-type hPIV3-F from a previously derived plasmid carrying the wild-type hPIV3-F ORF from strain Texas/12084/1983. PCR amplification was performed using primers carrying Fsel (forward primer) and Sbfl (reverse primer) restriction site sequences. The C-terminal 51 amino acids, corresponding to amino acids 489-539 of SEQ ID NO: 9, were deleted in order to generate a soluble, secreted protein. The HL1 construct was generated by changing every 3rd codon in the wild-type sequence to either guanine or cytosine. No change was made for codons in which the 3rd base pair was already guanine or cytosine. Gene synthesis was performed by DNA 2.0. This product was subsequently used as a template for PCR amplification and subcloning into pCLD. Primers for PCR amplification and subcloning of wild-type and GC enriched (HL1) hPIV3-F constructs into the pCLD vector are presented in Table 6 below. Additional PCR primers (presented in Table 6) for wild-type and GC enriched (HL1) hPIV3-F constructs were used to generate constructs containing His tags.

TABLE 5

| Soluble hPIV-F | | % GC | % GC3 | SEQ |
|---|---|---|---|---|
| Construct | Synthesized by | Content | Content | ID NO |
| Wild-type | PCR | 36 | 29 | 10 |
| HL1 | DNA 2.0 | 60 | 100 | 11 |
| Wild-type 6xHis tag | PCR | 36 | 30 | 13 |
| HL1 6xHis tag | DNA 2.0 and PCR | 60 | 100 | 14 |

TABLE 6
Primer sequences for hPIV3-F amplification and cloning

| | 5′Primer Sequences | 3′Primer Sequences |
|---|---|---|
| Wild-type soluble | 5′-gcatggccggccatgccaacttc | 5′-gcatcctgcaggttaatgccaa |
| construct | aatactgctaattattacaac-3' (SEQ | tttccaatggaatctag-3' (SEQ ID |
| | ID NO: 30) | NO: 31) |
| GC-enriched soluble | 5'-gcaggccggccatgccgacgtc | 5'-gcacctgcaggttagtgccag |
| construct (HL1) | catcctgc-3' (SEQ ID NO: 32) | ttgccgatggag-3' (SEQ ID NO: |
| | | 33) |
| Wild-type soluble | 5'-gcatggccggccatgccaacttc | 5'-gcatcctgcaggttagtgatggtg |
| 6xHis tag | aatactgctaattattacaac-3' (SEQ | atggtggtgatgccaatttccaatgga |
| | ID NO: 34) | atctag-3' (SEQ ID NO: 35) |
| HL1 soluble 6xHis | 5'-gcaggccggccatgccgacgtc | 5'-gcatcctgcaggttagtgatgg |
| tag | catcctgc-3' (SEQ ID NO: 36) | tgatggtggtggtgccagttgccga |
| | | tggag-3' (SEQ ID NO: 37) |

Each sequence-confirmed DNA plasmid was prepared by the Qiagen MaxiPrep procedure (Qiagen) and quantitated via Nanodrop (Thermo-Fisher Scientific) analysis. Plasmids were subsequently linearized by restriction digest cleavage using Sapl enzyme (New England Biolabs) that has only one cleavage site near the Ampicillin resistance ORF and hence is far removed from both the fusion protein and glutamate synthetase coding regions. Linearized plasmid DNA was ethanol-precipitated, quantified by Nanodrop analysis, and subsequently used for transfection of suspension CHO (e.g. CAT-S) cells.

2. Cell Culture Conditions

In order to optimize RSV-F expression, production cells able to efficiently generate high titers of soluble RSV-F protein facilitating both research studies and vaccine development were chosen. To this end, CAT-S cells were employed as they can be cultured with animal component free media and supplements ensuring pre-GMP conditions are met. Additionally, utilizing a cell line grown without serum would simplify the purification process by minimizing unwanted proteins from culture media.

CAT-S suspension CHO cells (Accession No. 10090201) were maintained in chemically defined CHO media (CD-CHO; Invitrogen) supplemented with 6 mM L-glutamine (Invitrogen). Culture components were determined to be animal component free to ensure quality assurance requirements were met for future clinical grade recombinant protein production. Cells were cultured in 5% CO₂ at 37° C. in a humidified shake incubator set at 140 rpm to ensure continuous and adequate mixing of the suspension cultures. Cell growth and viability were determined every 3-4 days by ViCell analysis (Beckman Coulter Instruments) and generally ranged from 95-98% viability for the parental CAT-S cell line. Growth of CAT-S cells on site mimicked in parallel the growth of CAT-S cells obtained from another site relative to both cell viability and density. Cells were sustained in 5% CO₂ at 37° C.

Invitrogen's suspension CHO-S cells were maintained in CD-CHO media supplemented with 8 mM L-glutamine. CHO-S cells were often grown using the same conditions as those described above for CAT-S cells. In some instances, both CAT-S and CHO-S cell lines were sub-cultured upon reaching densities of ˜2×10⁶ viable cells per ml, approximately every 3-4 days, at which point the cells were split 1:10 (final concentration 0.2×10⁶ cells/mL) into fresh media. The culture reagents were obtained from Invitrogen.

3. Transfections

Suspension CHO cultures, CAT-S and CHO-S, were harvested at greater than 90% viability by centrifugation at 300 g for 10 min. 100 μl aliquots of CAT-S (and in some cases CHO-S) cells (1×10⁸ cells/ml) in cGMP grade Amaxa Nucleofector Solution V (Amaxa) was mixed with 2 μg linearized DNA (described above). In some instances, pelleted 1×10⁷ cells per transfection were resuspended in 100 μL of Amaxa Nucleofector Solution V per transfection and added to Nucleofection cuvette containing 10 μg linearized plasmid DNA (described above). Cuvettes were subsequently and individually placed into the Amaxa Nucleofection apparatus and electroporated at Program #U024 that has been previously determined by MedImmune. Cells were immediately diluted into 50 ml pre-warmed (37° C.) CD-CHO media containing no L-glutamine. A 50 μl aliquot of the cell solution was added to each well of ten 96-well plates and incubated statically overnight under 5% CO2 humidified conditions to promote cell recovery. The following day 150 μl of selection media, CD-CHO containing Methionine sulfoximine (MSX; Sigma), was added to each well. The final concentration of MSX for selection was 65 μM. Cells were incubated statically for 21 days to allow for colonies to develop. Cell cultures were analyzed for percent viability after ˜21 days selection in the MSX selective media and supernatants were removed and normalized for viable cell density of each transfectant (2×10⁶ viable cells per mL). Subsequently, wells with single colonies were identified and a 20 μl aliquot of culture media removed from each well for protein quantification via Western blot analysis (run under both reducing and non-reducing conditions) and/or quantitative ELISA to determine expression of RSV or PIV3 fusion proteins.

4. Western Blot Analysis

To evaluate sRSV-F production by CAT-S clonal cell lines, culture media was removed from plates and centrifuged for 10 min at 300 g to remove residual cells or cellular debris. Resultant supernatants were mixed 3 volumes media to 1 volume 4×SDS sample buffer (Invitrogen) and loaded onto 4-12% Tris-glycine Novex gels (Invitrogen) for non-reduced samples. For determination of reduced protein banding patterns, samples were incubated at 70° C. for 15 minutes with 10% reducing solution (DTT, Invitrogen) prior to loading on 4-12% Tris-Glycine gels. Protein gels were run for 1 hour at 150 volts at room temperature. Separated proteins were transferred to PVDF membrane and then blocked in PBS containing 5% skim milk powder. Blots were incubated first with polyclonal goat anti-RSV antibody (Millipore MAB1128) and then with rabbit anti-goat HRP conjugated secondary (Dako). Protein bands were visualized after incubation with the SuperSignal ECL substrate (Pierce) and exposure to Kodak Biomax Film. Lanes were loaded with equivalent quantities of cell culture supernatant so comparisons could be made across multiple clones derived from each sRSV-F construct.

In some instances, Western blots were performed according to the following protocol.

a. Reducing SDS-PAGE

Samples were diluted in Laemmli buffer containing β-mercaptoethanol, boiled for 10 minutes and run on 12% Tris-glycine gels (Invitrogen). Proteins were transferred to PVDF using the XCell-II Blot system (Invitrogen) and probed using either Motavizumab (Medimmune) or goat anti-RSV (Millipore) antibody. The blots were probed with HRP-conjugated anti-human and anti-goat antibodies respectively. Signal was detected using ECL Plus (GE Biosciences) and autoradiography film.

b. Non-Reducing SDS-PAGE

Samples were diluted with ¼ volume of 4×LDS loading buffer (Invitrogen) and loaded directly onto 4-12% Tris-glycine gradient gels (Invitrogen). Separated proteins were transferred to PVDF (0.45 μM pore diameter) using the XCell-II Blot system (Invitrogen) and then probed using either Motavizumab (Medimmune) or goat anti-RSV (Millipore) antibody. The blots were probed with HRP-conjugated anti-human and anti-goat antibodies respectively. Signal was detected using SUPERSIGNAL Femto ECL reagent (Pierce) and autoradiography film.

5. ELISA Screening

Wells containing transfected cells with colony growth were screened for sRSV-F expression by quantitative ELISA using a research-purified protein as standard. Briefly, 96 well Immulon 4B plates (high binding) were coated overnight at 4° C. with 1:1000 dilution of polyclonal goat anti-RSV antibody (Millipore MAB1128) in carbonate-bicarbonate buffer (pH 9.0). Plates were washed repeatedly with phosphate buffered saline containing 0.05% TWEEN 20 (PBS-TWEEN) before plates were blocked with 200 μl Superblock solution (Pierce) for 1 hour at room temperature. ELISA plates were washed repeatedly with PBS-TWEEN before 1:10 dilutions (10% Superblock in PBS) of cell culture media, removed from wells exhibiting colony growth, were added. Sample plates were incubated 2 hours at room temperature before being washed and incubated with a 1:1000 dilution of the primary detection antibody, monoclonal mouse anti-RSV (MAB8262 from Millipore). Subsequently, unbound primary antibody was removed, plates were incubated with anti-mouse HRP secondary antibody (Dako), and then were developed using TMB substrate. Development of TMB was stopped by addition of HCl and plates assayed for 450 nm absorbance on a SpectraMax 5 plate reader using SoftMax Pro software (Molecular Devices). The A450 values of individual samples were compared to a standard curve produced from b/hPIV3-expressed and research purified sRSV-F.

In some instances, the following ELISA protocol was used. Supernatant media from each transfected cell population was removed and normalized to viable cell density (2×10⁶ viable cells per mL). Supernatant samples were then analyzed for expression of RSV fusion proteins by enzyme-linked-immunosorbent assay (ELISA). Supernatants were diluted 1:2 from neat samples (starting media) to 1:2048, tested in duplicate wells of 96-well plates, and compared to a standard curve derived from purified sRSV-F of known amount. Briefly, plates were coated overnight with 1 μg/mL Motavizumab (in PBS) then blocked for 1 hour with 300 μL/well Superblock (Pierce) prior to addition of test samples. In some instances, 150 ng Motavizumab in a 100 μl volume was used to coat each well. The sRSV-F bound was subsequently detected using a 1:1000 dilution of monoclonal mouse anti-RSV antibody (MAB8262; Millipore) followed by 1:10000 rabbit anti-mouse peroxidase and addition of the peroxidase substrate TMB. Plates were read on a SpectraMax plate reader and the sRSV-F present in each sample was calculated from a standard curve prepared from purified sRSV-F diluted 1:3 from 4 μg/mL to 0.2 ng/mL.

In some instances, a development-purified form of b/hPIV3-expressed sRSV-F (0.4 mg/ml) with more stringent purity and quantification was used to generate the standard curve (range 4 to 0.031 ng/ml). These improvements were implemented and subsequently provided greater CV values and lower error rate, i.e. greater confidence, in the sRSV-F production levels assayed.

6. Cell Culture Scale-Up (CAT-S Cells)

Initial 96-well colonies were ranked relative to recombinant protein expression levels and those clones with detectable protein were transferred to 24 well plates for cell expansion. ELISAs and Western blots were iteratively repeated on 24 well plate cultures prior to transfer to 6 well culture plates and finally to 125 Erlenmeyer shake flasks with 10 ml selective culture media. Approximately one week following inoculation into Erlenmeyer flasks, cultured cells were moved from static incubators to shake incubators and began mixing at 80 rpm for 7 days. ViCell analysis for cell density and viability was determined for each shake flask culture following one week of gentle shaking. Shaking was then increased to 100 rpm for 4 days and finally to the optimized maintenance speed of 140 rpm thereafter. Protein expression and cell density/viability in shake flasks were analyzed periodically to determine productivity and growth in order for the ranking of individual clones to be determined. Clones with less than 10% of the expression level of the top clone were periodically dropped during the scale-up process to select for the top producing cultures.

Overgrowth culture production was determined in both fed and unfed batch conditions by inoculating shake flasks with 0.2×10⁶ cells per ml selective media and sampling for protein expression and cell growth periodically. The MedImmune culture media CDC-3 (chemically defined CHO-3) was used for growth of CAT-S in bioreactors and the MedImmune M20A feed was used for supplemental feeding of fed-batch cultures.

7. Flow Cytometry

CAT-S and CHO-S cell cultures were harvested by centrifugation at 300 g for 10 min prior to staining for RSV-F protein expression. Cell pellets containing ˜3×10⁶ viable cells per sample were resuspended in 500 μL fixation solution (Becton Dickinson Cytofix/Cytoperm kit; San Diego, Calif.) and incubated overnight at 4° C. Fixed cells were permeabilized with Becton Dickinson's Cytoperm solution according to manufacturer's protocol and then were incubated with 1 μg/mL Motavizumab for 1 hour at 4° C. Cells were washed to remove unbound Motavizumab and subsequently incubated for 1 hour at 4° C. with 1:1000 dilution of anti-human FITC conjugated detection antibody (Dako Cytomation). Cells were then washed and analyzed on either the LSR II or FacsCalibur flow cytometry instruments and expression analyzed via FACSDIVA or CELLQUEST software respectively.

### Example 2

### Optimization of Full-Length RSV-F Expression

To evaluate the impact nucleotide sequence can have on the expression of the full-length RSV-F protein, cell lines were transfected with DNA constructs containing either wild-type, codon-optimized, or GC-enriched versions of the full-length RSV-F nucleotide sequence. The nucleotide constructs encoding identical RSV-F proteins, were cloned into the pCMVscript expression vector and transfected into BSRT7, MRC-5, and Vero cells according to the methods set forth in Example 1. The effects of nucleotide sequence variations and cis-acting nuclear export elements on full-length RSV-F protein expression are described below.

A. Recombinant RSV-F Protein Expression Levels are Improved by Increased GC Abundance and Cis-Acting Nuclear Export Elements

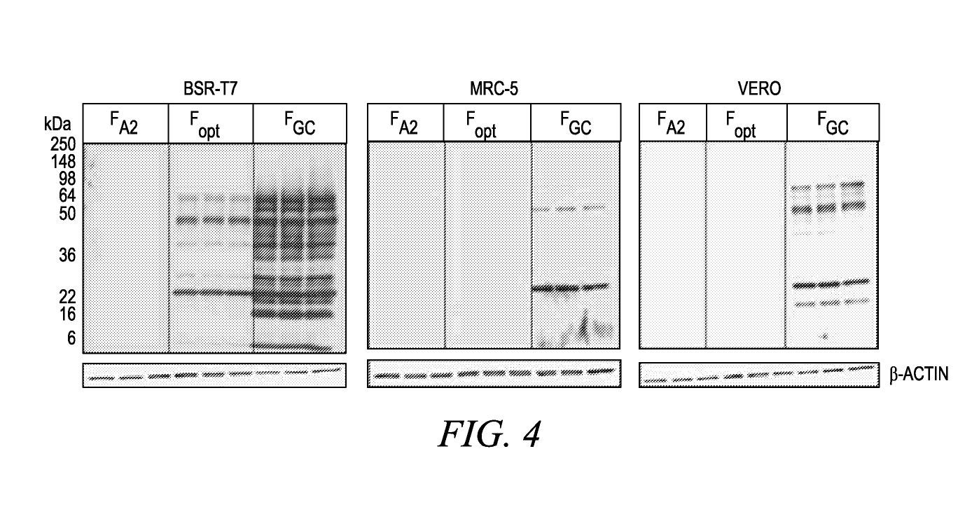

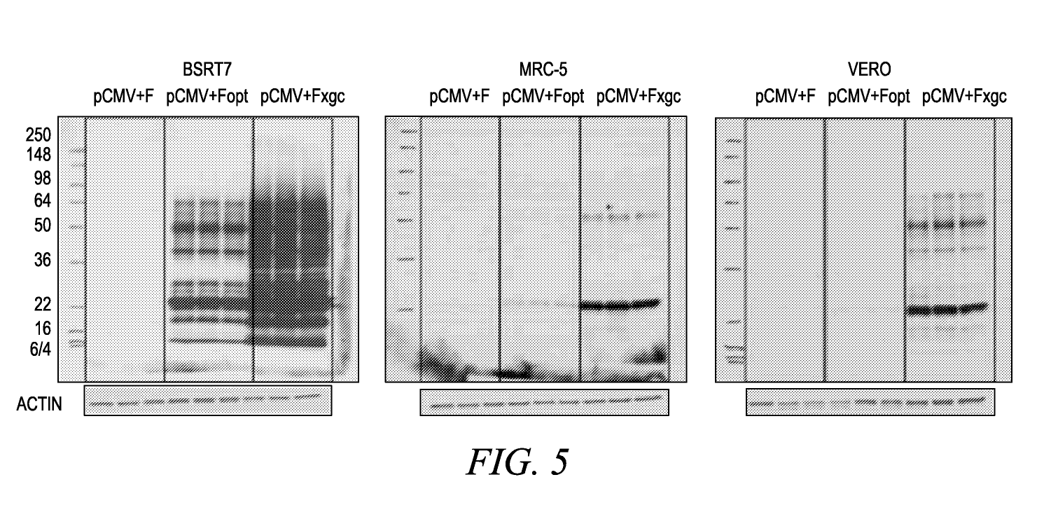

The first test was performed to determine whether increasing GC abundance to overcome the negative effect that the AU-rich nature of viral transcripts have on nuclear export could improve recombinant F protein expression levels. The wild-type RSV A2 F gene sequence (F_{A2}), codon-optimized RSV A2 F sequence (Fₒₚₜ), and RSV A2 F sequence enriched in the percentage of G or C nucleotides but coding for the same amino acid sequence (F_{GC}) were cloned into the pCMVscript expression vector. These RSV-F sequences differ in GC content by more than 10% as shown in FIG. 1B. Each expression vector was transfected into BSR-T7, MRC-5, or Vero cells. These cell types were chosen because they are routinely used to rescue recombinant viruses, propagate live attenuated RSV vaccines, and propagate RSV, respectively. Western blots for F protein expression were performed on cell lysates collected at 36 h post-transfection, and were normalized to β-actin. F protein was detected using a high affinity monoclonal antibody (Wu et al., 2007 J. Mol. Bio. 368, 652-655) that recognizes the F₁ fragment of F protein, which migrates to 48 kDa, and consistently recognizes another smaller unidentified fragment that migrates at ˜22 kDa. The expression level of Fₒₚₜ was greater than native F_{A2} in BSR-T7 cells, but this observation could not be detected in MRC-5 and Vero cells (FIGS. 4 and 5). Improving GC abundance of the F protein transcript (F_{GC}) resulted in improved F protein expression levels in all cell types compared to F_{A2} and Fₒₚₜ (FIGS. 4 and 5). These results raise the possibility that the effect of codon-optimization of the RSV-F protein gene sequence for recombinant expression from standard mammalian expression vectors is, in part, the improvement of GC abundance.

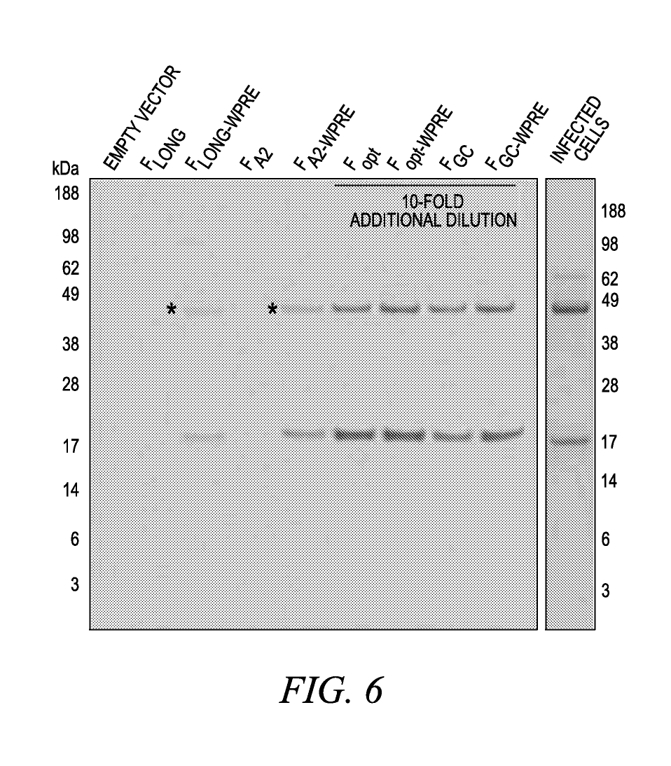

To further evaluate whether recombinant expression of F protein is hindered by poor nuclear export, WPRE was introduced into the pEBNA F protein expression vectors as shown in FIG. 1A. The same molar amounts of each expression vector were transfected into 293F cells, which were chosen because they are highly permissive to transfection. In some experiments, a vector encoding secreted luciferase was cotransfected with the pEBNA F protein expression vectors and aliquots of the supernatants were harvested at 24 h following transfection. Assays for luciferase activity in these supernatants showed that the transfection efficiencies were similar for all constructs. Cell lysates were collected 48 h post transfection and western blots were performed using the same high affinity antibody to detect F protein that was used in FIGS. 4 and 5. The lysates were first diluted to normalize for protein loading based on β-actin and these normalized lysates were further diluted prior to loading so that the lysates that showed greater F protein expression were diluted 10-fold more than the lysates with less F protein expression for ease of comparison (FIG. 6). The same trend was observed with the pEBNA vector clones as was observed with the pCMVscript vector clones (FIGS. 4 and 5), where F protein expression was improved by codon-optimization and by increased GC optimization (FIG. 6). F protein expression was enhanced by the addition of WPRE to the F_{Long} and F_{A2} expression vectors, as observed by the appearance of a clear, faint band at 48 kDa representing the F₁ portion of the F protein and the apparent 22 kDa band that is only detected by western blot upon F protein expression (FIG. 6). The enhancement by WPRE was less evident when WPRE was included in the Fₒₚₜ and F_{GC} expression vectors (FIG. 6). For comparison purposes, a Western blot performed in the same manner with a lysate from RSV infected HEp-2 cells is shown (FIG. 6). The same two bands were detected by the F protein specific antibody, indicating that the unidentified band is not a particular result of recombinant F protein expression (FIG. 6). The observation that WPRE and increased GC abundance improves F protein expression suggests the low recombinant expression level of F protein is likely due, at least in part, to inefficient nuclear export of F protein transcripts.

B. Increased GC Abundance does not Improve Recombinant F Protein Expression in the Context of a Cytoplasmic Recombinant Virus Expression Vector

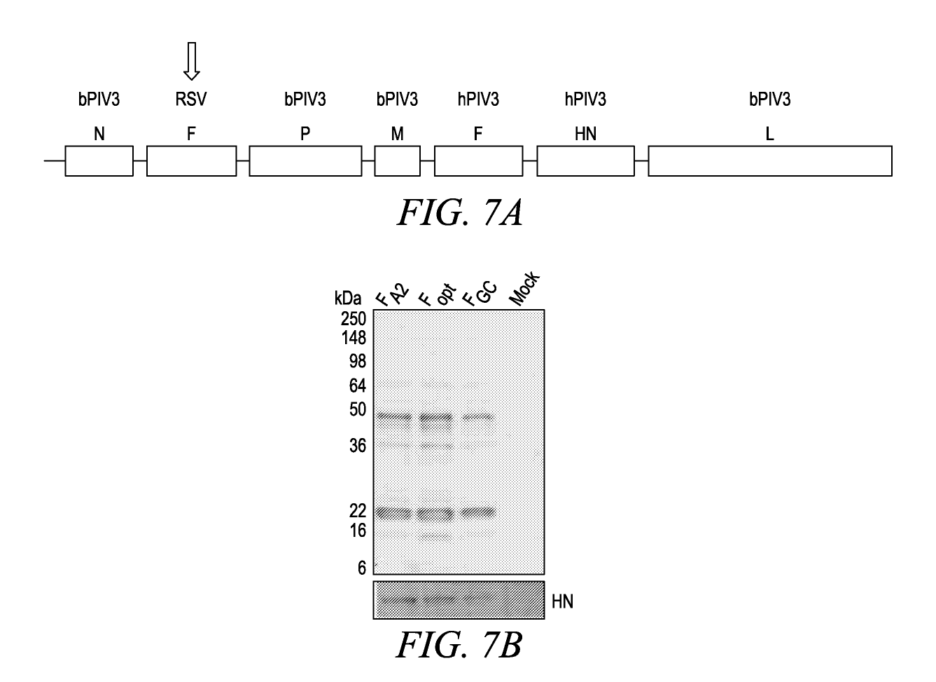

If the low level of F protein expression from standard mammalian expression vectors is the result of poor nuclear export, the RSV-F expression levels of the F_{A2}, Fₒₚₜ and F_{GC} should be approximately the same when transcription occurs in the cytoplasm. However, if the improvement to F protein expression by codon-optimization or increased GC abundance of the F gene is not the result of improved nuclear export, the expression levels of Fₒₚₜ and F_{GC} should be higher than the wild-type F_{A2} level when transcription occurs in the cytoplasm. Therefore, F_{A2}, Fₒₚₜ and F_{GC} were expressed from b/h PIV3, a recombinant virus expression vector derived from bovine parainfluenza virus type 3 (bPIV3) and described previously (Tang et al., 2003 J. Virol. 77, 10819-10828) to test this scenario (FIG. 7A). Expression of the RSV-F gene from b/h PIV3 occurs in the cytoplasm of infected cells in a similar manner as expression of RSV-F gene in RSV infected cells. As shown in FIG. 7A, the RSV-F gene was cloned into the second gene position between the bPIV3 N and P genes. Lysates from Vero cells infected with b/h PIV3/RSV-F recombinant viruses expressing different versions of the F gene were collected at 48 h post-infection for Western blotting. Western blot analysis showed increasing GC abundance did not improve F protein expression levels in the context of this virus vector (FIG. 7B). These data further indicate poor nuclear export contributes to the low level of F protein expression from standard plasmid-based mammalian expression vectors.

C. Premature Polyadenylation Contributes to the Low Level of Recombinant RSV-F Protein Expression

In addition to assessing nuclear export, a test was performed to determine whether recombinant F protein transcription by RNA polymerase II, as opposed to the viral polymerase that normally transcribes this gene, would result in transcripts that have undergone spurious splicing and/or premature polyadenylation. To determine whether RNA polymerase II derived transcripts of the F gene are spliced, total RNA was purified from 293F cells 48 h post-transfection with the same molar amount of each pEBNA F protein expression vectors. RT-PCR that spanned the transcriptional start site within the CMV_{IE} promoter through the stop codon of the F protein was performed on each sample. Gel analysis of the RT-PCR showed the full-length transcript is the major transcript expressed in 293F cells transfected with all versions of the F gene. Therefore, splicing is not a major factor that contributes to the low level of recombinant F protein expression.

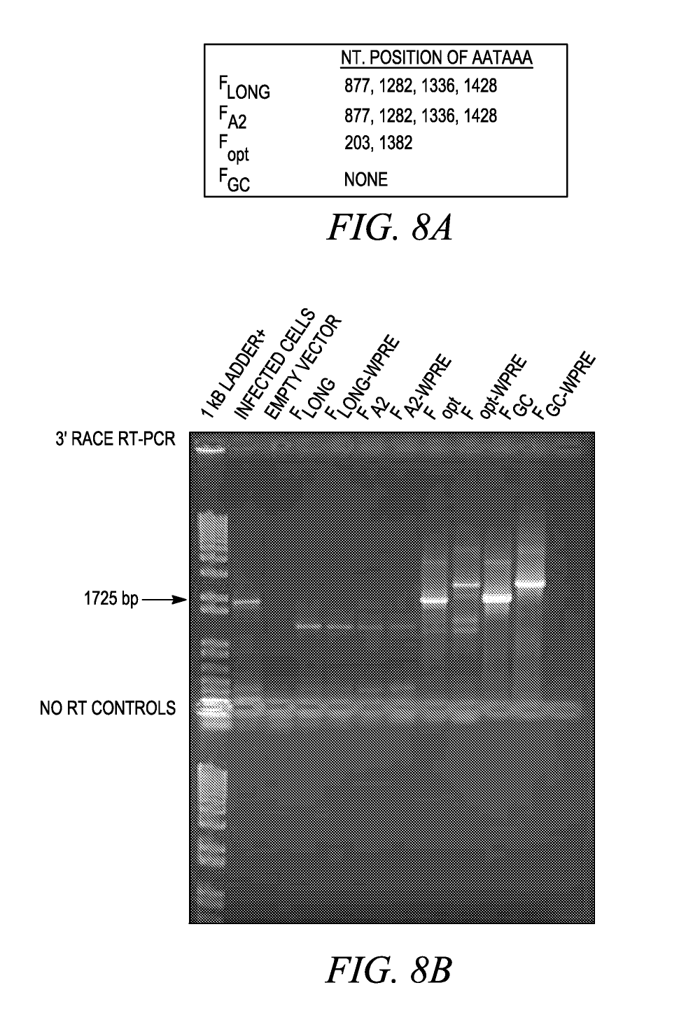

Premature polyadenylation of F gene transcripts during recombinant expression was previously reported (Ternette et al., 2007 Virol. J. 4, 51). RACE analysis is routinely used to characterize either 5′ or 3′ ends of transcripts by identifying transcript start sites, as well as, transcript end sites. Therefore, 3′ RACE analysis was used to test for premature polyadenylated F transcripts. Based on polyadenylation signal sequences identified in the F_{Long}, F_{A2}, and Fₒₚₜ sequences, it was anticipated these genes could undergo premature polyadenylation, whereas the F_{GC} sequence would not undergo premature polyadenylation due to the lack of polyadenylation signal sequences, as summarized in FIG. 8A. Total RNA was purified from 293F cells 48 h post-transfection with the same molar amount of each pEBNA F protein expression vector and subjected to 3′ RACE analysis. The 3′ RACE PCR was designed to amplify the full-length F transcript, which should be approximately 1,700 nucleotides in length for transcripts without WPRE and approximately 2,325 nucleotides in length for transcripts with WPRE. The 3′ RACE analysis revealed that most of the F protein transcripts detected in 293F cells transfected with the F_{Long} and F_{A2} expression vectors were smaller than 1,700 or 2,325 nucleotides, as quantitated by densitometry of the gel (FIG. 8B). It is likely that full-length transcripts were present in these samples, as full-length transcripts were detected by the PCR analysis for splicing and full-length F protein production was detected by western blot in cells transfected with F_{Long} and F_{A2} protein expression vectors that included WPRE (FIG. 6), but were below the limit of detection by the 3′ RACE analysis. In contrast, the smaller transcripts detected in 293F cells transfected with the Fₒₚₜ expression vector were considerably less abundant (3 and 40% for Fₒₚₜ and F_{opt-WPRE}) respectively). The size of these smaller transcripts correlates to the polyadenylation signal sequences in F_{Long}, F_{A2}, and Fₒₚₜ (FIG. 8A). The premature polyadenylation was specific for RNA polymerase II, as these truncated transcripts were not detected during viral replication (FIG. 8B) where the F protein is transcribed by the viral polymerase. In addition to revealing truncated transcripts, the 3′ RACE RT-PCR demonstrates that there were differences in transcript abundance. The same amount of RNA went into each RT step of the RACE analysis, yet only 0.5 μl of first strand reaction went into the second strand synthesis for Fₒₚₜ and F_{GC} samples compared to 2 μl for the F_{Long} and F_{A2} samples to achieve the results shown in FIG. 8B. Therefore, the abundance of F_{Long} and F_{A2} transcripts appeared to be lower than Fₒₚₜ and F_{GC} transcripts.

The major 3′ RACE PCR products were cloned into TOPO TA pCR2.1 and sequenced to identify the presence and precise location of polyadenylation events. The sequencing results confirmed that the F_{Long} and F_{A2} truncated transcripts were the result of premature polyadenylation events that correlated to a polyadenylation signal sequence located at nucleotide position 1282 within the F gene, whereas full-length transcripts were identified for Fₒₚₜ and F_{GC}. These results indicate that premature polyadenylation contributes to the low level of recombinant F protein expression and the codon-optimization (Fₒₚₜ) was not enough to fully counteract this problem. However, increasing the GC content (F_{GC}) resulted in the elimination of all the premature polyadenylation signal sequences, AATAAA, and better expression of recombinant RSV-F protein.

D. Increased Recombinant RSV-F Protein Expression Correlates to Greater Fusion Activity

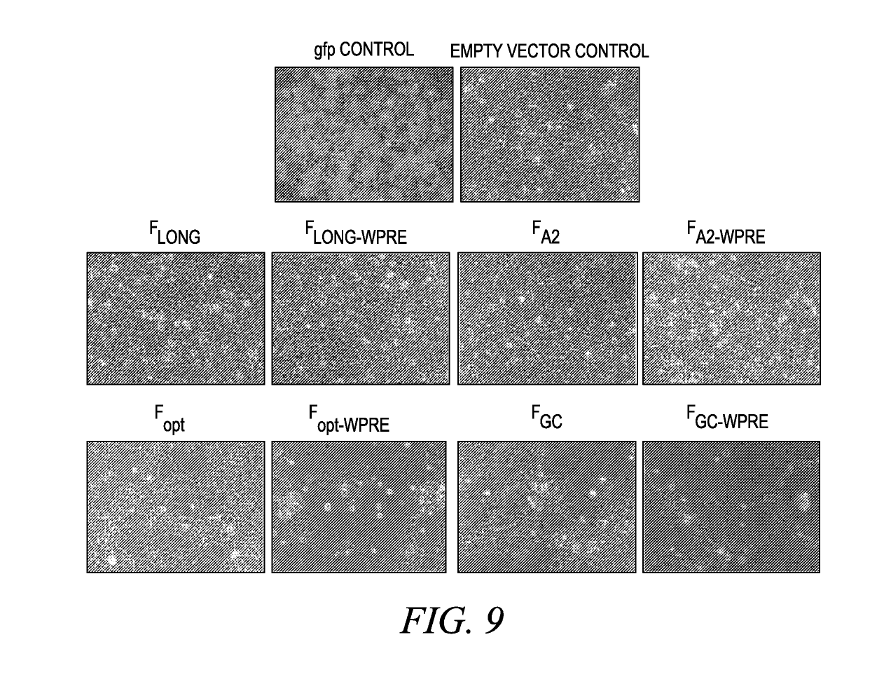

To determine if improved recombinant F protein expression impacts function, both transient and stable expression approaches were used to assess F protein-mediated cell-to-cell fusion, or syncytium formation. For transient expression, 293Ad cells were transfected with the same molar amount equivalents of each pEBNA F protein expression vector. F protein-mediated cell-to-cell fusion was analyzed 24 h post-transfection by microscopy. Visual inspection showed the extent of syncytium formation (FIG. 9) correlated well with increased expression levels of F protein (FIGS. 4-6). The most extensive syncytium formations were consistently observed in cells transfected with the F_{GC-WPRE} expression vector (FIG. 9). To analyze stable expression of F protein, 293-derived cell lines were created that express F_{GC} or F_{GC-WPRE} upon tetracycline induction. An assay to determine the amount of an antibody specific for RSV-F protein (Wu et al., 2007 J. Mol. Biol. 368, 652-655) bound to these cell lines was developed. At 44 h post induction, there was an average 6.31-fold±0.58 (SEM) greater amount of antibody bound to the F_{GC-WPRE} cell line compared to the F_{GC} cell line, indicating a greater amount of F protein expression. This difference in the level of F protein expression had a significant impact on F protein function as syncytium formation was only observed for the F_{GC-WPRE} cell line and not for the F_{GC} cell line.

### Example 3

### Production of Soluble RSV-F Protein in CAT-S and CHO-S Cells

To evaluate the impact nucleotide sequence nucleotide sequence can have on the expression of a soluble version of the RSV-F protein (sRSV-F) and to determine which cell line is optimal for sRSV-F protein production, CAT-S and CHO-S cell lines were nucleofected with DNA constructs containing various versions of the sRSV-F nucleotide sequence. Specifically, the following constructs were used: soluble wild-type (SEQ ID NO: 3), soluble codon-optimized (OPT; SEQ ID NO: 4), soluble GC rich (GC3; SEQ ID NO: 5), soluble medium GC3 (HL2; SEQ ID NO: 6) and soluble GH5; which encode identical sRSV-F amino acid sequences (SEQ ID NO: 7). The sRSV-F nucleotide constructs used in this example encode the ectodomain of the RSV-F protein. The nucleotide sequence encoding carboxy terminal 50 amino acids containing the transmembrane and internal domains of the protein were deleted from each construct, as described in Example 1. Each sRSV-F construct, subcloned into the pCLDv4550-synthetic MCS-poly A expression vector, was Amaxa Nucleofected into CAT-S and CHO-S cells according to the methods described in Example 1. Cells were cultured according to the methods described in Example 1 and the cultures were assayed for sRSV-F protein using Western Blot, ELISA and FACS.

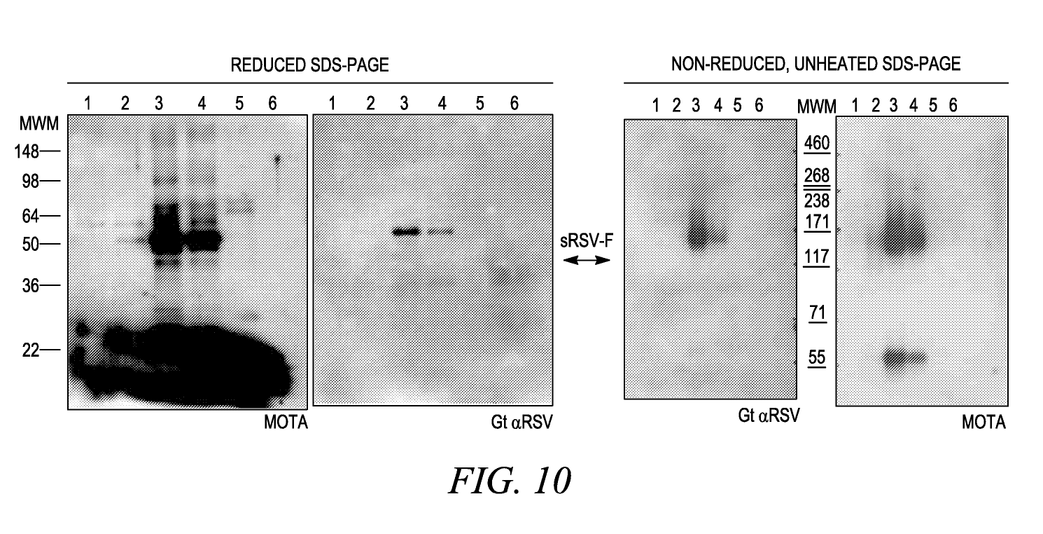

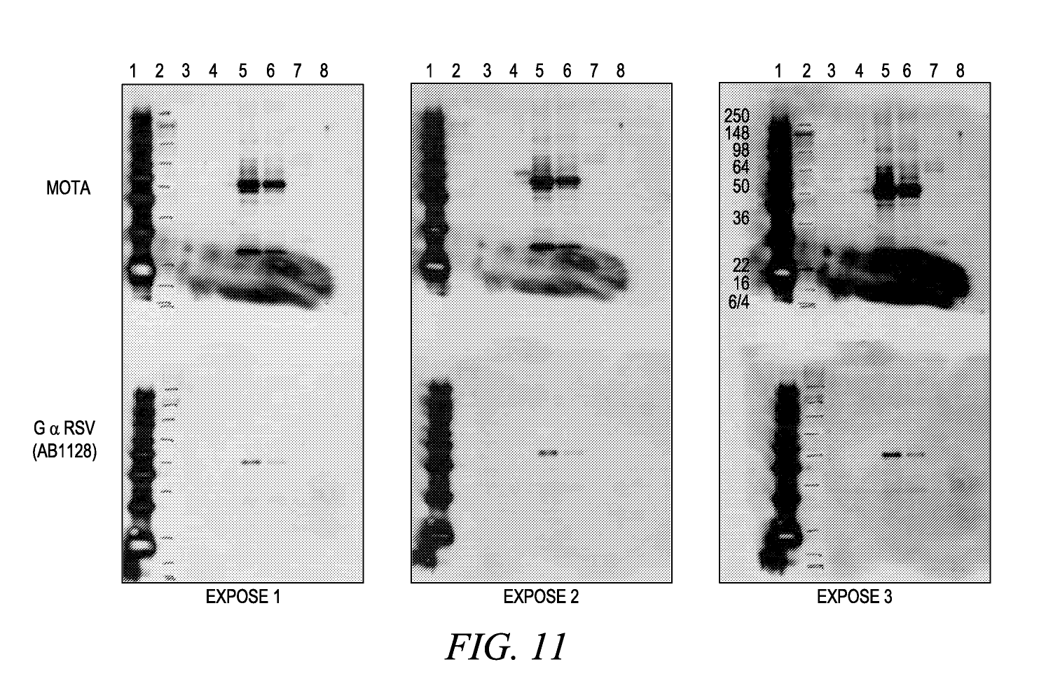

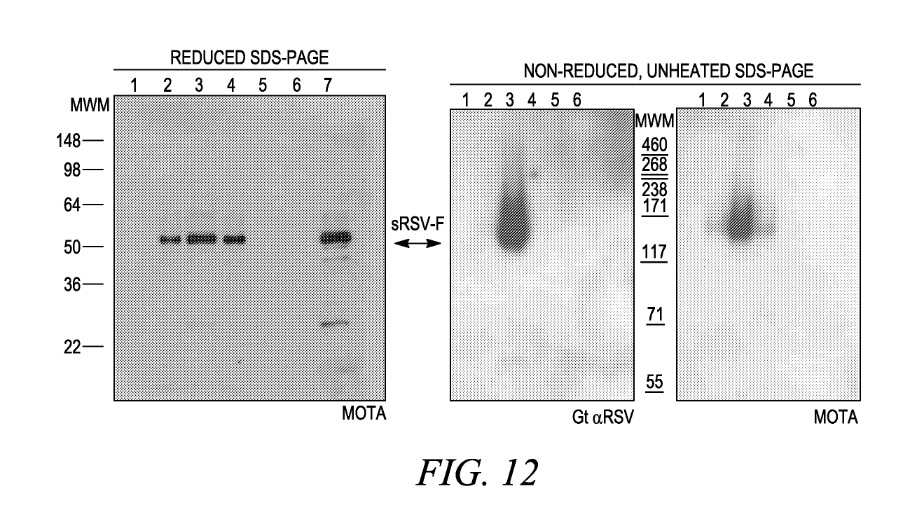

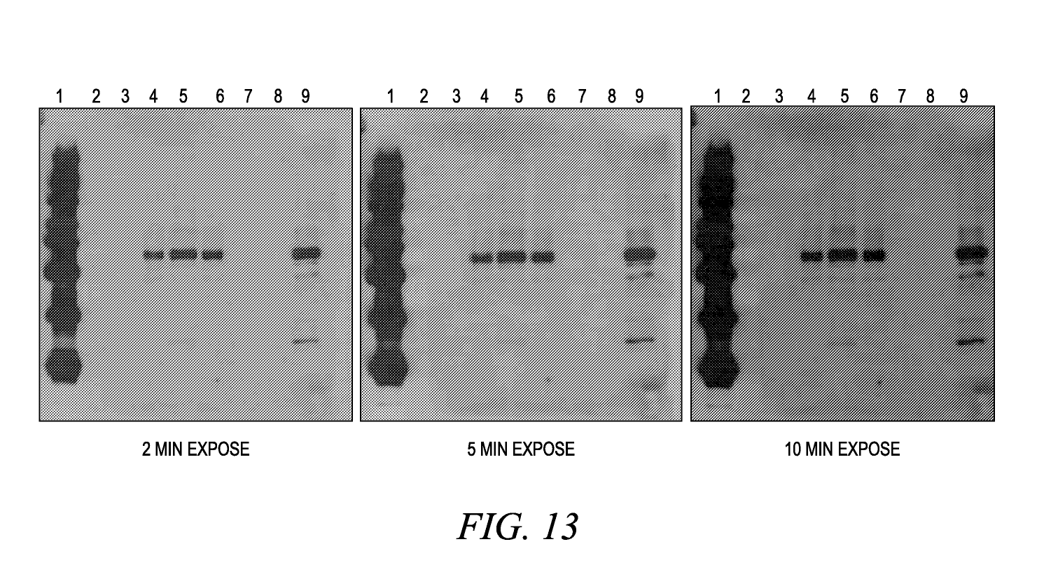

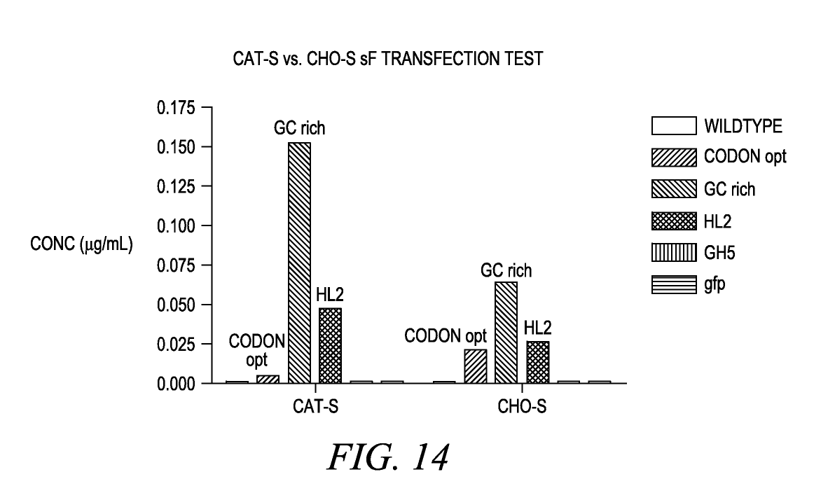

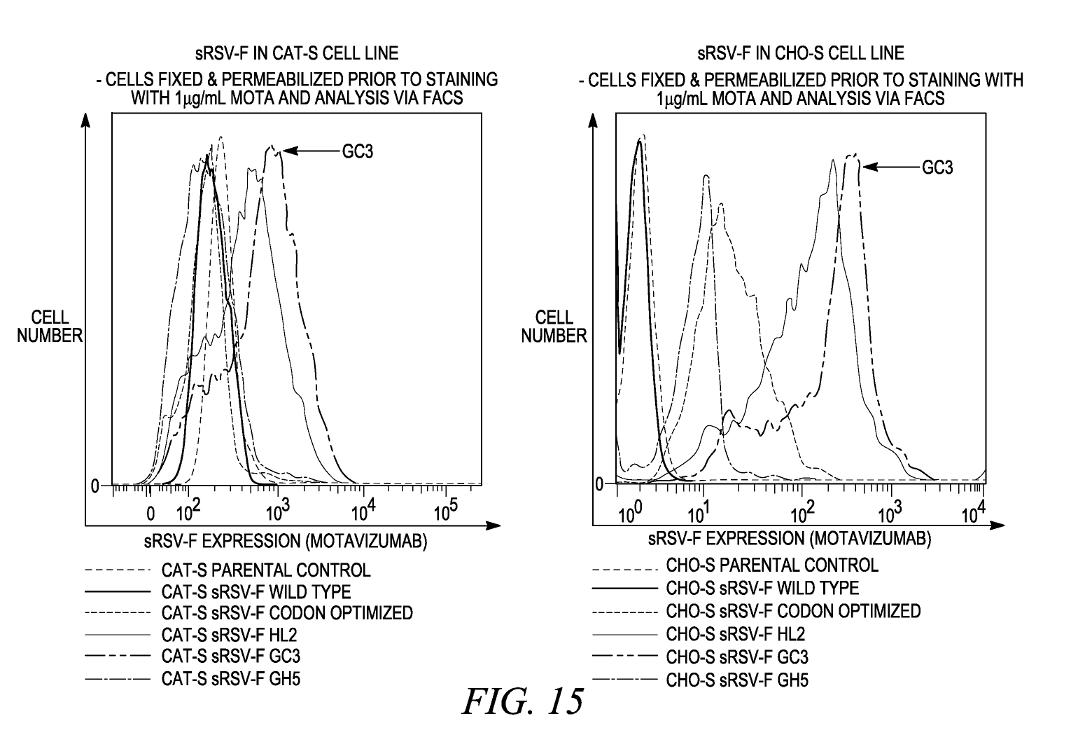

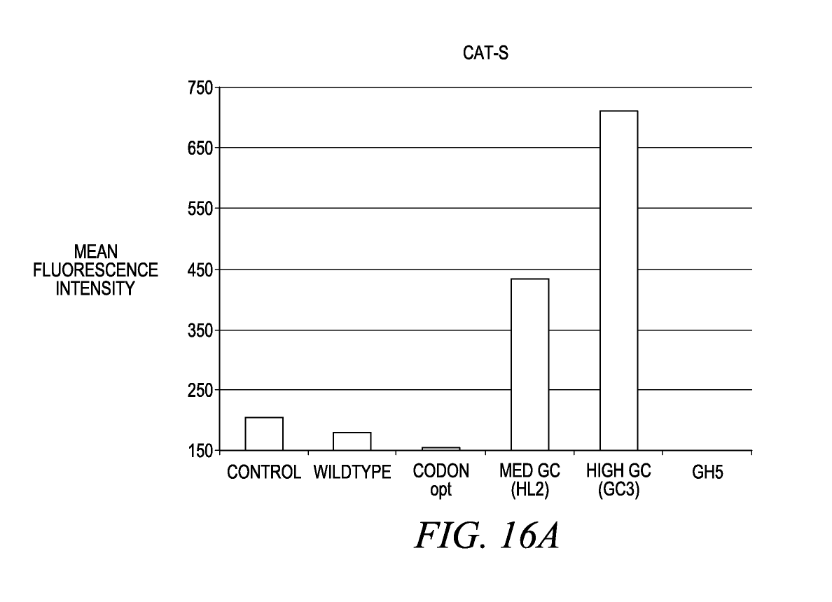

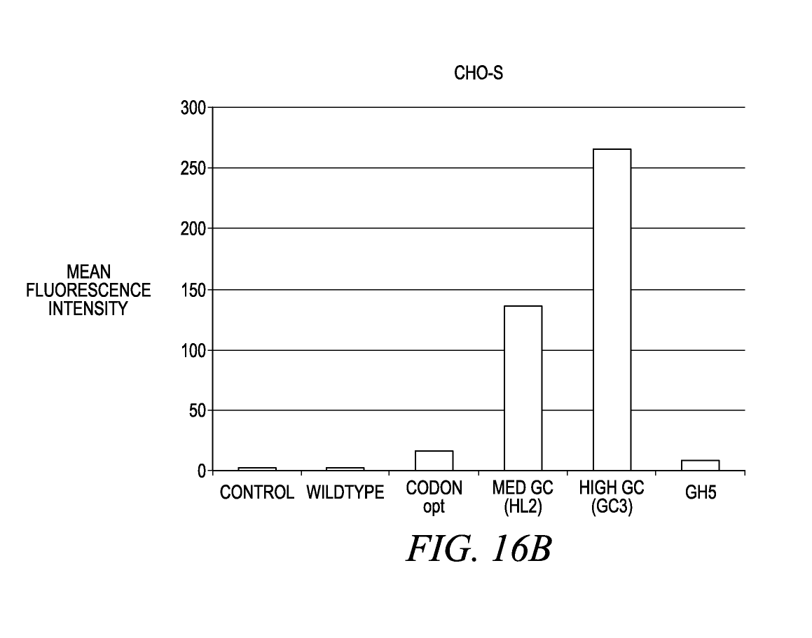

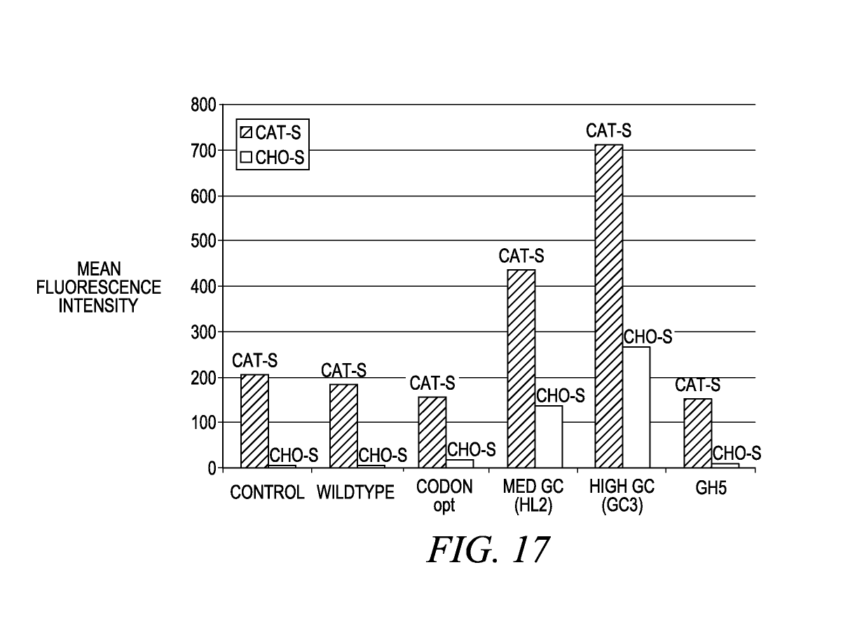

Western blots were performed according to the methods presented in Example 1. Western blot results for sRSV-F production in CAT-S cells are presented in FIGS. 10 and 11. Western blot results for sRSV-F production in CHO-S cells are presented in FIGS. 12 and 13. ELISA analysis was performed according to the methods presented in Example 1. ELISA results for sRSV-F production in CAT-S and CHO-S cells are presented in FIG. 14. According to the ELISA analysis, expression of sRSV-F protein was the highest in CAT-S cells transfected with the high-GC construct (GC3), followed by the medium GC (HL2) and codon optimized constructs. Overall protein production was greater than 2 fold higher in the CAT-S cell line compared to the CHO-S cell line. FACS analysis was performed according to the methods presented in Example 1. FACS results for sRSV-F production in CAT-S and CHO-S cells are presented in FIGS. 15, 16, and 17. According to the FACS analysis, expression of sRSV-F protein was ranked highest following transfection with the high-GC construct (GC3), followed by mid-GC content (HL2), regardless of which cell line was utilized. The wild-type, codon optimized (OPT), and GH5 versions were not significantly above background levels.

### Example 4

### Optimization of Soluble RSV-F Nucleotide Sequence in CAT-S Cells

To evaluate the impact nucleotide sequence can have on the expression of a soluble version of the RSV-F protein (sRSV-F), CAT-S cell lines were nucleofected with DNA constructs containing either wild-type, codon-optimized, or GC-enriched versions of the sRSV-F nucleotide sequences (SEQ ID NOs: 3, 4, and 5, respectively), all of which encode identical sRSV-F amino acid sequences (SEQ ID NO: 7). All three sRSV-F nucleotide constructs encode the ectodomain of the RSV-F protein. The nucleotide sequence encoding carboxy terminal 50 amino acids containing the transmembrane and internal domains of the protein were deleted from each construct, as described in Example 1. Each sRSV-F construct, subcloned into the pCLDv4550-synthetic MCS-poly A expression vector, was Amaxa Nucleofected into CAT-S cells.

A. Stable, Clonal Cell Line Generation

The nucleofected CAT-S cells were diluted among 96-well plates for clonal cell line generation. Following 3 weeks of selective growth, colonies developed across the plates of each construct transfection. 127 colonies were manually identified among the wild-type soluble plates and of these 116 clones (91.3%) had colony morphologies representative of those produced from a single cellular clone. 111 colonies were identified among the codon-optimized plates of which 99 appeared to be the result of a single cellular clone (89.2%). Finally, only 27 wells had appreciable cell growth among the GC-enriched transfection plates although all of these colonies (100%) had the desired morphology of that arising from a single cell.

B. sRSV-F Protein Production and ELISA Screening

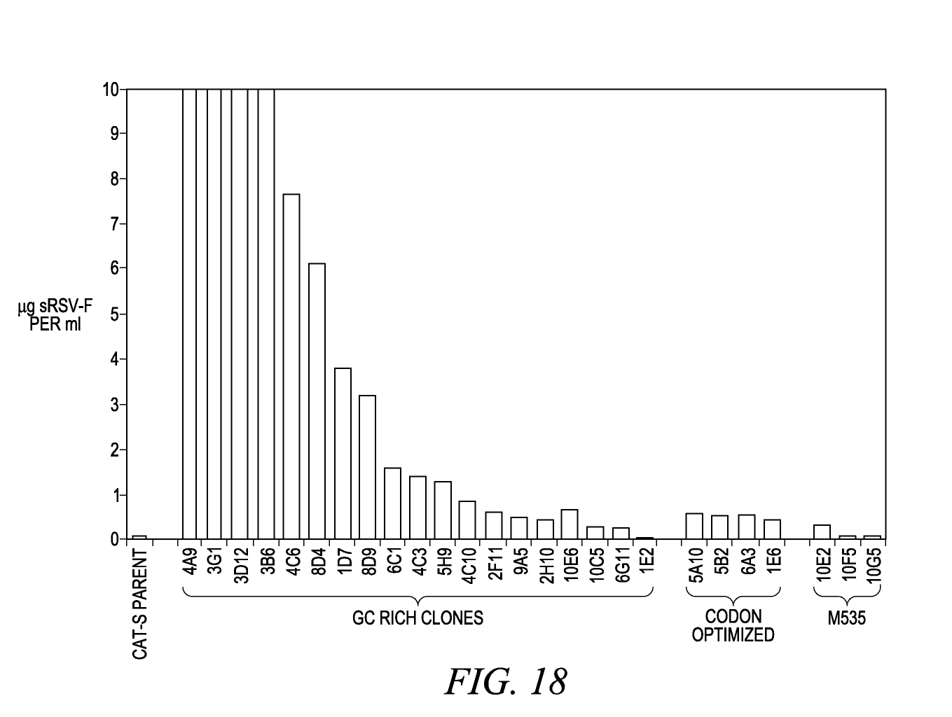

In this experiment, protein production was assayed for each sRSV-F nucleotide construct. 20 μA aliquots of culture media were removed from wells exhibiting growth from a single cellular clone, described in part A above, and tested for protein production via quantitative ELISA. sRSV-F protein levels among colonies expressing transcripts from the wild-type soluble construct were generally below or at best bordered the level of detection (˜0.1 μg/mL). Recombinant expression levels improved slightly in codon-optimized clones (range ˜0.2 to 1.1 μg/mL), but were strongest for GC-enriched clones (range 0.1 to greater than 10 μg/mL) many of which were above the standard curve utilized in the assay (FIG. 18).

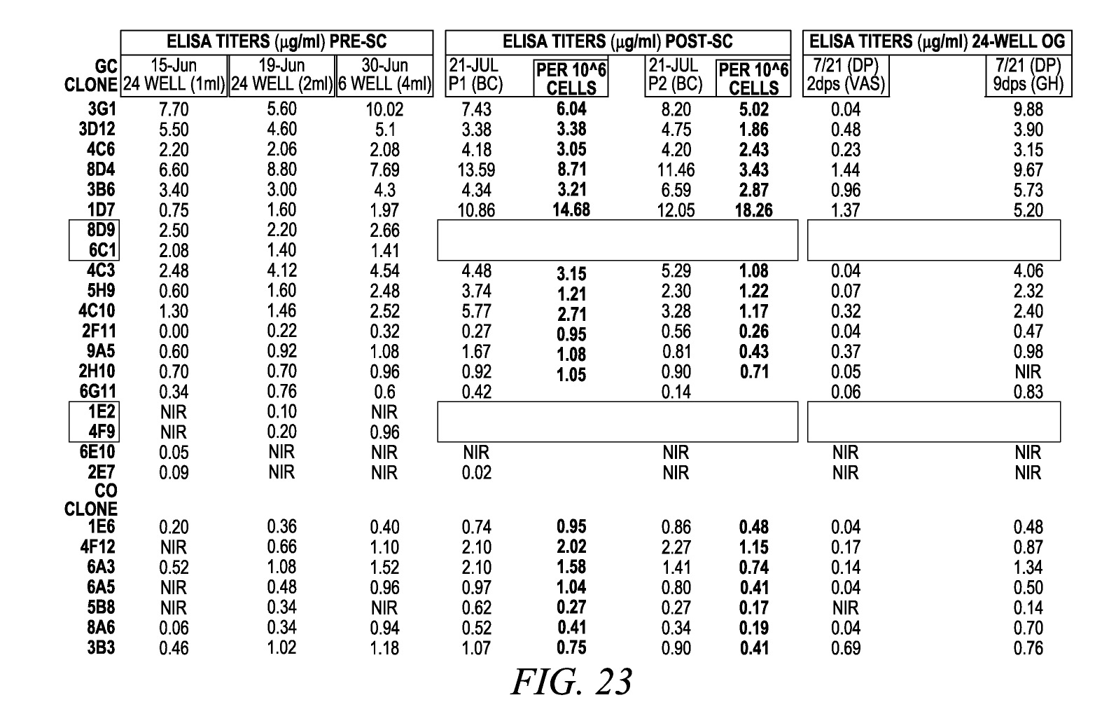

Cells from all wells expressing detectable sRSV-F levels were scaled-up into 24 well culture plates and then to 6 well plates over the course of 21-28 days, depending on cell growth rate. At multiple time-points within this scale-up process sRSV-F production was assayed by ELISA. The amounts of sRSV-F protein produced at various time points are presented in FIG. 23.

The dominant production clones were solely and consistently found among cells transfected with the GC-enriched construct at every time-point assayed. sRSV-F levels produced by codon-optimized clones were modest in comparison to GC-enriched lines and marginally detected among the wild-type soluble lines. As a result, the wild-type soluble lines were systematically dropped from the study and only GC-enriched and codon-optimized parental lines were continued forward into development.

C. sRSV-F Protein Production in Selected Parental Clones

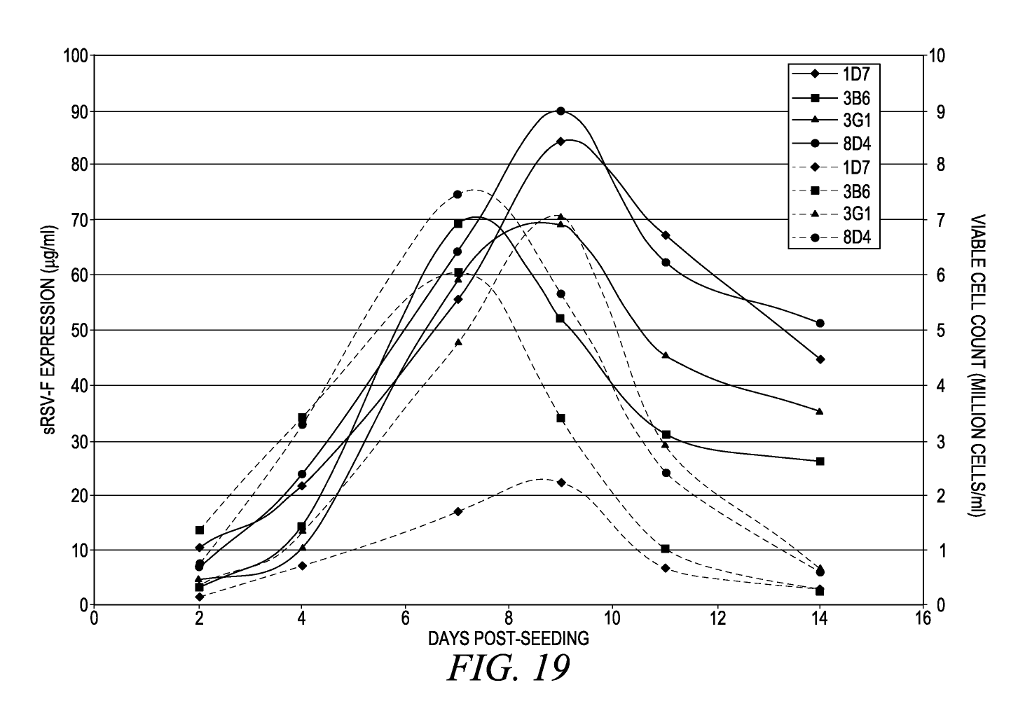

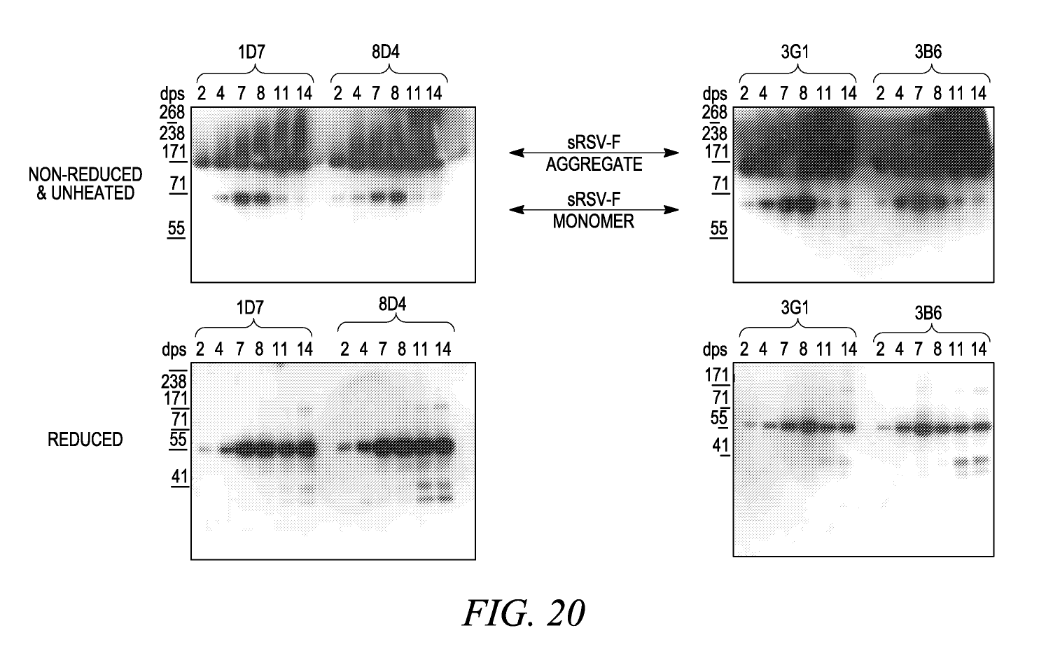

Based upon both expression level of sRSV-F and cell growth characteristics, parental clones were ranked for production capability and the top 22 clones (specifically, GC clones 3G1, 3D12, 4C6, 8D4, 3B6, 1D7, 4C3, 5H9, 4C10, 2F11, 9A5, 2H10, 6G11, 6E10, and 2E7, and CO clones 1E6, 4F12, 6A3, 6A5, 5B8, 8A6, and 3B3) were put into shake flask culture at 140 rpm. These conditions are optimal for growth of the suspension CAT-S line and were expected to boost production titers and cell growth levels higher than those obtained by static plate growth. The top 9 parental lines were subsequently placed in overgrowth shake flask culture to assess those likely to be the best producers. This manner of culture seeded clonal cells at identical levels on day 0 (0.2×10⁶ cells per ml) and assayed both cell viability/density and sRSV-F production over 14 days of further culture with no additional media change or feed. This method allowed assessment of how individual lines fared in response to growth stress. As cell density peaked at approximately day 7 it was anticipated media constituents became limiting for cell growth. Expression titers and viability data for the top four clones are illustrated in FIG. 19, revealing sRSV-F levels up to 90 μg/ml was attained. These results represent levels at least 6 fold higher than that achievable in Vero cell bioreactors, the previously utilized optimal production system. Shake flask overgrowth results were further confirmed via Western blot analysis (FIG. 20).

D. Bioreactor sRSV-F Protein Production

Bioreactor and fed-batch bioreactor data have demonstrated greater than 500 μg/ml sRSV-F production levels achieved with the 8D4 parental subclone, greater than 340 μg/ml sRSV-F production levels for clone 3B6, greater than 280 μg/ml sRSV-F production levels for clone 3G1. Further data have demonstrated about 1.3 mg/ml sRSV-F production levels for clone 1D7 and about 1.6 mg/ml sRSV-F production levels for clone 8D4.

### Example 5

### hPIV3-F Production in CAT-S Cells

To determine whether the high GC3 approach can be applied to other viral glycoproteins besides RSV-F, production of hPIV3-F in CAT-S cells using modified nucleotide sequence constructs was tested. The hPIV3-F constructs used in this Example include soluble wild-type (SEQ ID NO: 10), soluble high GC (HL1; SEQ ID NO: 11), His-tagged soluble wild-type (SEQ ID NO: 13), and His-tagged high GC (SEQ ID NO: 14). The high GC construct was generated as a 100% GC3 construct, with each codon containing a guanine or a cytosine at every third base pair. A 6×his-tag was added due to a paucity of anti-hPIV3-F antibodies available for use in Western blotting. The hPIV3-F constructs were cloned into the pCLD plasmid using methods described for RSV-F in Example 1 and transfected into CAT-S cells. Cells were cultured as described and Western blots were performed on the CAT-S transfectant lysates.

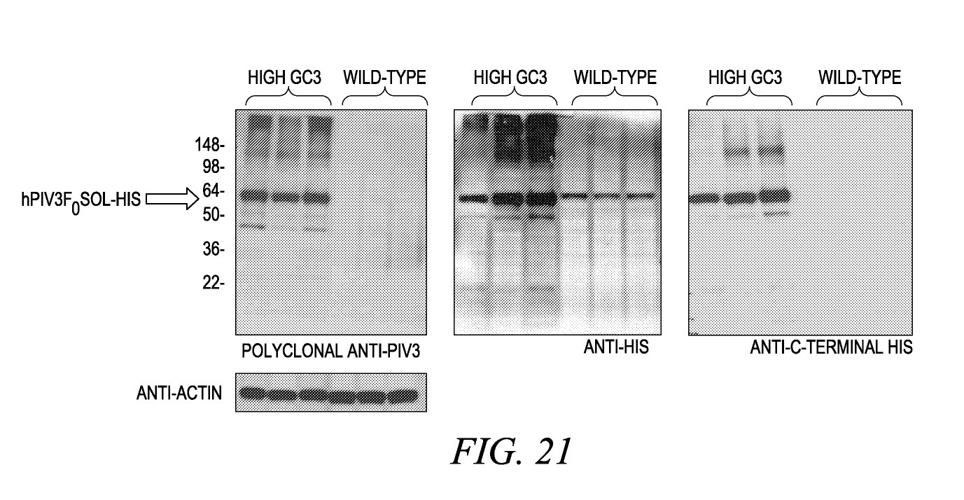

Western blots were performed on CAT-S transfectant lysates taken from three independent wells (innoculated with cells from each transfection mixture) using a polyclonal anti-P1V3 antibody (VMRD P1-3 virus (bovine) antiserum, goat origin lot G197/031306), anti-HIS antibody (Sigma catalog #H1029 Monoclonal anti-polyhistidine antibody produced in mouse), and anti-C-terminal HIS antibody (Invitrogen catalog #R930-25 Anti-his(Cterm) antibody). The expected size of the his-tagged F0 precursor protein was approximately 55 kDa and the his-tagged F1 protein was approximately 40 kDa. The Western blot results are presented in FIG. 21. The results show that high-GC3 hPIV3-F expression is significantly higher than that of the wild-type sequence. Therefore this approach can be applied to viral glycoproteins other than RSV-F.

### Example 6

### Examples of Sequences

Provided hereafter are non-limiting examples of certain nucleotide and amino acid sequences.

TABLE 7
Examples of Sequences

| | | | | | | |
|---|---|---|---|---|---|---|
| | | % | | | | |
| | | iden- | GC | GC3 | SEQ | |
| | | tity | con- | con- | ID | |
| Name | Type | to wt* | tent | tent | NO | Sequence |

RSVA2

| | | | | | | |
|---|---|---|---|---|---|---|
| WT (full- | NA | — | 35 | 30 | 1 | atggagttgctaatcctcaaagcaaatgcaattaccacaatcctcactgcagtc |
| length) | | | | | | acattttgttttgcttctggtcaaaacatcactgaagaattttatcaatcaaca |
| | | | | | | tgcagtgcagttagcaaaggctatcttagtgctctgagaactggttggtatacc |
| | | | | | | agtgttataactatagaattaagtaatatcaagaaaaataagtgtaatggaaca |
| | | | | | | gatgctaaggtaaaattgataaaacaagaattagataaatataaaaatgctgta |
| | | | | | | acagaattgcagttgctcatgcaaagcacacaagcaacaaacaatcgagccaga |
| | | | | | | agagaactaccaaggtttatgaattatacactcaacaatgccaaaaaaaccaat |
| | | | | | | gtaacattaagcaagaaaaggaaaagaagatttcttggttttttgttaggtgtt |
| | | | | | | ggatctgcaatcgccagtggcgttgctgtatctaaggtcctgcacctagaaggg |
| | | | | | | gaagtgaacaagatcaaaagtgctctactatccacaaacaaggctgtagtcagc |
| | | | | | | ttatcaaatggagtcagtgtcttaaccagcaaagtgttagacctcaaaaactat |
| | | | | | | atagataaacaattgttacctattgtgaacaagcaaagctgcagcatatcaaat |
| | | | | | | atagaaactgtgatagagttccaacaaaagaacaacagactactagagattacc |
| | | | | | | agggaatttagtgttaatgcaggtgtaactacacctgtaagcacttacatgtta |
| | | | | | | actaatagtgaattattgtcattaatcaatgatatgcctataacaaatgatcag |
| | | | | | | aaaaagttaatgtccaacaatgttcaaatagttagacagcaaagttactctatc |
| | | | | | | atgtccataataaaagaggaagtcttagcatatgtagtacaattaccactatat |
| | | | | | | ggtgttatagatacaccctgttggaaactacacacatcccctctatgtacaacc |
| | | | | | | aacacaaaagaagggtccaacatctgtttaacaagaactgacagaggatggtac |
| | | | | | | tgtgacaatgcaggatcagtatctttcttcccacaagctgaaacatgtaaagtt |
| | | | | | | caatcaaatcgagtattttgtgacacaatgaacagtttaacattaccaagtgaa |
| | | | | | | gtaaatctctgcaatgttgacatattcaaccccaaatatgattgtaaaattatg |
| | | | | | | acttcaaaaacagatgtaagcagctccgttatcacatctctaggagccattgtg |
| | | | | | | tcatgctatggcaaaactaaatgtacagcatccaataaaaatcgtggaatcata |
| | | | | | | aagacattttctaacgggtgcgattatgtatcaaataaaggggtggacactgtg |
| | | | | | | tctgtaggtaacacattatattatgtaaataagcaagaaggtaaaagtctctat |
| | | | | | | gtaaaaggtgaaccaataataaatttctatgacccattagtattcccctctgat |
| | | | | | | gaatttgatgcatcaatatctcaagtcaacgagaagattaaccagagcctagca |
| | | | | | | tttattcgtaaatccgatgaattattacataatgtaaatgccggtaaatccacc |
| | | | | | | acaaatatcatgataactactataattatagtgattatagtaatattgttatca |
| | | | | | | ttaattgctgttggactgctcttatactgtaaggccagaagcacaccagtcaca |
| | | | | | | ctaagcaaagatcaactgagtggtataaataatattgcatttagtaactaa |
| WT (full- | AA | — | — | — | 2 | mellilkanaittiltavtfcfasgqniteefyqstcsavskgylsalrtgwyt |
| length) | | | | | | svitielsnikknkcngtdakvklikqeldkyknavtelqllmqstpatnnrar |
| | | | | | | relprfmnytlnnakktnvtlskkrkrrflgfllgvgsaiasgvavskvlhleg |
| | | | | | | evnkiksallstnkavvslsngvsvltskvldlknyidkqllpivnkqscsisn |
| | | | | | | ietviefqqknnrlleitrefsvnagvttpvstymltnsellslindmpitndq |
| | | | | | | kklmsnnvqivrqqsysimsiikeevlayvvqlplygvidtpcwklhtsplctt |
| | | | | | | ntkegsnicltrtdrgwycdnagsvsffpqaetckvqsnrvfcdtmnsltlpse |
| | | | | | | vnlcnvdifnpkydckimtsktdvsssvitslgaivscygktkctasnknrgii |
| | | | | | | ktfsngcdyvsnkgvdtvsvgntlyyvnkqegkslyvkgepiinfydplvfpsd |
| | | | | | | efdasisqvnekingslafirksdellhnvnagksttnimittiiiviivills |
| | | | | | | liavglllyckarstpvtlskdqlsginniafsn |
| Codon | NA | 73 | 46 | 58 | 16 | atggaacttcttattctcaaagccaatgcgattacaacaatccttactgctgta |
| optimized | | | | | | accttctgcttcgcatctggacagaatatcaccgaggaattctatcaatccacc |
| (full- | | | | | | tgcagcgcggtgtcaaaggggtatctttccgcattgagaacaggttggtataca |
| length) | | | | | | tccgttattactattgagctgtctaacatcaagaagaataaatgtaatggaact |
| | | | | | | gacgcaaaagtgaagctgatcaagcaggagcttgataagtacaaaaacgctgtg |
| | | | | | | acagaactccagctcctcatgcagagcaccccggcgacgaacaatagagcgcgg |
| | | | | | | cgcgagctgcctaggtttatgaattatacccttaacaacgctaagaagacaaac |
| | | | | | | gtgacgctctcaaagaagaggaaacgaaggtttcttggattcctgctcggggtg |
| | | | | | | ggatccgctattgcaagcggcgtggcggtttcaaaggtcctccacctggagggg |
| | | | | | | gaagtgaacaagattaagtcagcactcctgagtacaaacaaagcagtggtttct |
| | | | | | | ctgagcaacggagtgtcagtattgacgagcaaggtgcttgacctcaagaactac |
| | | | | | | attgacaaacagctgctgcccatagtgaacaaacagtcatgctccatctccaat |
| | | | | | | atcgagacagtcatcgaattccagcagaagaacaacagactcctggaaatcaca |
| | | | | | | cgggagtttagcgtgaatgcgggcgtaacaactcccgtgtccacctacatgctg |
| | | | | | | acaaattctgagctgctgagtctgataaatgatatgcctattacaaatgaccag |
| | | | | | | aagaagttgatgtccaacaatgtgcaaatagtcagacagcagtcttatagtatt |
| | | | | | | atgagcatcatcaaagaggaagttcttgcctatgttgtacaactgcccctctac |
| | | | | | | ggggtcatcgacacaccctgttggaagctgcacacctcacctctgtgcaccacc |
| | | | | | | aacacgaaagagggtagcaacatctgtctgactaggactgacaggggttggtac |
| | | | | | | tgcgataacgccggtagcgtgtcatttttcccacaagcagagacttgtaaagta |
| | | | | | | cagtccaacagggtcttttgtgacacaatgaattctcttaccctgcccagcgaa |
| | | | | | | gttaatctgtgtaacgtcgatatctttaatccaaagtacgattgtaaaatcatg |
| | | | | | | acatctaaaaccgatgtgagcagcagcgttattacaagtcttggcgctatcgtc |
| | | | | | | agctgttacggaaaaaccaagtgcacggcatccaacaagaatagaggcattata |
| | | | | | | aagaccttcagtaatgggtgtgactacgttagcaataagggcgtagacaccgtc |
| | | | | | | tccgtaggaaacacactgtactatgtaaataaacaagaaggcaaatccctttat |
| | | | | | | gtgaagggggagcctatcattaatttctacgaccctctggttttcccgagtgac |
| | | | | | | gagttcgatgccagcatatcccaagtgaatgagaaaatcaaccagtccttggcc |
| | | | | | | tttataaggaaaagcgatgagcttctgcacaacgtgaatgccggtaaatccacc |
| | | | | | | acaaacataatgatcaccactatcattatcgtcattattgtgatcttgctgagc |
| | | | | | | ctcatcgctgtggggctcctcttgtattgcaaagcccgctcaaccccagtcact |
| | | | | | | ctctctaaagaccaactgtctgggatcaataacatagccttttcaaattag |
| Codon | AA | 100 | — | — | 2 | See above |
| optimized | | | | | | |
| (full- | | | | | | |
| length) | | | | | | |
| High GC | NA | 76 | 58 | 100 | 17 | atggagttgctcatcctcaaggccaacgccatcaccacgatcctcacggccgtc |
| (GC rich | | | | | | acgttctgcttcgcgtccggccagaacatcaccgaggagttctaccagtcgacg |
| full- | | | | | | tgcagcgccgtgagcaagggctacctcagcgcgctgaggacgggctggtacacc |
| length) | | | | | | agcgtcatcacgatcgagttgagcaacatcaagaagaacaagtgcaacggcacc |
| | | | | | | gacgcgaaggtcaagttgatcaagcaggagttggacaagtacaagaacgccgtg |
| | | | | | | accgagttgcagttgctcatgcagagcacgccggcgacgaacaaccgcgccagg |
| | | | | | | agggagctcccgaggttcatgaactacacgctcaacaacgccaagaagaccaac |
| | | | | | | gtgaccttgagcaagaagaggaagaggaggttcctcggcttcttgttgggcgtc |
| | | | | | | ggctcggccatcgccagcggcgtggccgtctcgaaggtcctgcacctggagggc |
| | | | | | | gaggtgaacaagatcaagagcgcgctgctctccacgaacaaggccgtcgtcagc |
| | | | | | | ttgtccaacggcgtcagcgtcttgaccagcaaggtgttggacctcaagaactac |
| | | | | | | atcgacaagcagttgttgccgatcgtgaacaagcagagctgcagcatctcgaac |
| | | | | | | atcgagaccgtgatcgagttccagcagaagaacaacaggctgctcgagatcacc |
| | | | | | | agggagttcagcgtcaacgccggcgtcacgacgccggtcagcacctacatgttg |
| | | | | | | accaacagcgagttgttgtccttgatcaacgacatgccgatcaccaacgaccag |
| | | | | | | aagaagttgatgtccaacaacgtgcagatcgtcaggcagcagagctactcgatc |
| | | | | | | atgtccatcatcaaggaggaggtcttggcctacgtcgtgcagttgccgctgtac |
| | | | | | | ggcgtcatcgacacgccctgctggaagctgcacacgtccccgctgtgcacgacc |
| | | | | | | aacacgaaggaggggtccaacatctgcttgaccaggaccgacaggggctggtac |
| | | | | | | tgcgacaacgccggctccgtgtcgttcttcccgcaggccgagacctgcaaggtc |
| | | | | | | cagtccaaccgcgtcttctgcgacacgatgaacagcttgacgttgccgagcgag |
| | | | | | | gtcaacctctgcaacgtcgacatcttcaaccccaagtacgactgcaagatcatg |
| | | | | | | acgtccaagaccgacgtcagcagctccgtgatcacgtcgctcggcgccatcgtg |
| | | | | | | tcctgctacggcaagaccaagtgcaccgcgtccaacaagaaccgcggcatcatc |
| | | | | | | aagacgttctcgaacgggtgcgactacgtctcgaacaagggggtggacaccgtg |
| | | | | | | tccgtcggcaacacgttgtactacgtcaacaagcaggagggcaagagcctctac |
| | | | | | | gtcaagggcgagccgatcatcaacttctacgacccgttggtcttcccctcggac |
| | | | | | | gagttcgacgcgtcgatctcgcaggtcaacgagaagatcaaccagagcctggcg |
| | | | | | | ttcatccggaagtccgacgagttgttgcacaacgtgaacgccggcaagtccacc |
| | | | | | | acgaacatcatgatcacgacgatcatcatcgtgatcatcgtgatcttgttgtcg |
| | | | | | | ttgatcgccgtcggcctgctcttgtactgcaaggccaggagcacgcccgtcacg |
| | | | | | | ctgagcaaggaccagctgagcggcatcaacaacatcgcgttcagcaactaa |
| High GC | AA | 100 | — | — | 2 | See above |
| (GC rich | | | | | | |
| full- | | | | | | |
| length | | | | | | |
| WT | NA | 99 | 35 | 31 | 3 | atggagttgctaatcctcaaagcaaatgcaattaccacaatcctcactgcagtc |
| Soluble | | | | | | acattttgttttgcttctggtcaaaacatcactgaagaattttatcaatcaaca |
| | | | | | | tgcagtgcagttagcaaaggctatcttagtgctctgagaactggttggtatacc |
| | | | | | | agtgttataactatagaattaagtaatatcaagaaaaataagtgtaatggaaca |
| | | | | | | gatgctaaggtaaaattgataaaacaagaattagataaatataaaaatgctgta |
| | | | | | | acagaattgcagttgctcatgcaaagcacaccagcaacaaacaatcgagccaga |
| | | | | | | agagaactaccaaggtttatgaattatacactcaacaatgccaaaaaaaccaat |
| | | | | | | gtaacattaagcaagaaaaggaaaagaagatttcttggttttttgttaggtgtt |
| | | | | | | ggatctgcaatcgccagtggcgttgctgtatctaaggtcctgcacctagaaggg |
| | | | | | | gaagtgaacaagatcaaaagtgctctactatccacaaacaaggctgtagtcagc |
| | | | | | | ttatcaaatggagtcagtgtcttaaccagcaaagtgttagacctcaaaaactat |
| | | | | | | atagataaacaattgttacctattgtgaacaagcaaagctgcagcatatcaaat |
| | | | | | | atagaaactgtgatagagttccaacaaaagaacaacagactactagagattacc |
| | | | | | | agggaatttagtgttaatgcaggtgtaactacacctgtaagcacttacatgtta |
| | | | | | | actaatagtgaattattgtcattaatcaatgatatgcctataacaaatgatcag |
| | | | | | | aaaaagttaatgtccaacaatgttcaaatagttagacagcaaagttactctatc |
| | | | | | | atgtccataataaaagaggaagtcttagcatatgtagtacaattaccactatat |
| | | | | | | ggtgttatagatacaccctgttggaaactacacacatcccctctatgtacaacc |
| | | | | | | aacacaaaagaagggtccaacatctgtttaacaagaactgacagaggatggtac |
| | | | | | | tgtgacaatgcaggatcagtatctttcttcccacaagctgaaacatgtaaagtt |
| | | | | | | caatcaaatcgagtattttgtgacacaatgaacagtttaacattaccaagtgaa |
| | | | | | | gtaaatctctgcaatgttgacatattcaaccccaaatatgattgtaaaattatg |
| | | | | | | acttcaaaaacagatgtaagcagctccgttatcacatctctaggagccattgtg |
| | | | | | | tcatgctatggcaaaactaaatgtacagcatccaataaaaatcgtggaatcata |
| | | | | | | aagacattttctaacgggtgcgattatgtatcaaataaaggggtggacactgtg |
| | | | | | | tctgtaggtaacacattatattatgtaaataagcaagaaggtaaaagtctctat |
| | | | | | | gtaaaaggtgaaccaataataaatttctatgacccattagtattcccctctgat |
| | | | | | | gaatttgatgcatcaatatctcaagtcaacgagaagattaaccagagcctagca |
| | | | | | | tttattcgtaaatccgatgaattattacataatgtaaatgccggtaaatccacc |
| | | | | | | acaaattaa |
| WT | AA | 100 | — | — | 7 | mellilkanaittiltavtfcfasgqniteefyqstcsavskgylsalrtgwyt |
| Soluble | | | | | | svitielsnikknkcngtdakvklikqeldkyknavtelqllmqstpatnnrar |
| | | | | | | relprfmnytlnnakktnvtlskkrkrrflgfllgvgsaiasgvavskvlhleg |
| | | | | | | evnkiksallstnkavvslsngvsvltskvldlknyidkqllpivnkqscsisn |
| | | | | | | ietviefqqknnrlleitrefsvnagvttpvstymltnsellslindmpitndq |
| | | | | | | kklmsnnvqivrqqsysimsiikeevlayvvqlplygvidtpcwklhtsplctt |
| | | | | | | ntkegsnicltrtdrgwycdnagsvsffpqaetckvqsnrvfcdtmnsltlpse |
| | | | | | | vnlcnvdifnpkydckimtsktdvsssvitslgaivscygktkctasnknrgii |
| | | | | | | ktfsngcdyvsnkgvdtvsvgntlyyvnkqegkslyvkgepiinfydplvfpsd |
| | | | | | | efdasisqvnekinqslafirksdellhnvnagksttn |
| Codon | NA | 74 | 46 | 58 | 4 | atggaacttcttattctcaaagccaatgcgattacaacaatccttactgctgta |
| Optimized | | | | | | accttctgcttcgcatctggacagaatatcaccgaggaattctatcaatccacc |
| Soluble | | | | | | tgcagcgcggtgtcaaaggggtatctttccgcattgagaacaggttggtataca |
| | | | | | | tccgttattactattgagctgtctaacatcaagaagaataaatgtaatggaact |
| | | | | | | gacgcaaaagtgaagctgatcaagcaggagcttgataagtacaaaaacgctgtg |
| | | | | | | acagaactccagctcctcatgcagagcaccccggcgacgaacaatagagcgcgg |
| | | | | | | cgcgagctgcctaggtttatgaattatacccttaacaacgctaagaagacaaac |
| | | | | | | gtgacgctctcaaagaagaggaaacgaaggtttcttggattcctgctcggggtg |
| | | | | | | ggatccgctattgcaagcggcgtggcggtttcaaaggtcctccacctggagggg |
| | | | | | | gaagtgaacaagattaagtcagcactcctgagtacaaacaaagcagtggtttct |
| | | | | | | ctgagcaacggagtgtcagtattgacgagcaaggtgcttgacctcaagaactac |
| | | | | | | attgacaaacagctgctgcccatagtgaacaaacagtcatgctccatctccaat |
| | | | | | | atcgagacagtcatcgaattccagcagaagaacaacagactcctggaaatcaca |
| | | | | | | cgggagtttagcgtgaatgcgggcgtaacaactcccgtgtccacctacatgctg |
| | | | | | | acaaattctgagctgctgagtctgataaatgatatgcctattacaaatgaccag |
| | | | | | | aagaagttgatgtccaacaatgtgcaaatagtcagacagcagtcttatagtatt |
| | | | | | | atgagcatcatcaaagaggaagttcttgcctatgttgtacaactgcccctctac |
| | | | | | | ggggtcatcgacacaccctgttggaagctgcacacctcacctctgtgcaccacc |
| | | | | | | aacacgaaagagggtagcaacatctgtctgactaggactgacaggggttggtac |
| | | | | | | tgcgataacgccggtagcgtgtcatttttcccacaagcagagacttgtaaagta |
| | | | | | | cagtccaacagggtcttttgtgacacaatgaattctcttaccctgcccagcgaa |
| | | | | | | gttaatctgtgtaacgtcgatatctttaatccaaagtacgattgtaaaatcatg |
| | | | | | | acatctaaaaccgatgtgagcagcagcgttattacaagtcttggcgctatcgtc |
| | | | | | | agctgttacggaaaaaccaagtgcacggcatccaacaagaatagaggcattata |
| | | | | | | aagaccttcagtaatgggtgtgactacgttagcaataagggcgtagacaccgtc |
| | | | | | | tccgtaggaaacacactgtactatgtaaataaacaagaaggcaaatccctttat |
| | | | | | | gtgaagggggagcctatcattaatttctacgaccctctggttttcccgagtgac |
| | | | | | | gagttcgatgccagcatatcccaagtgaatgagaaaatcaaccagtccttggcc |
| | | | | | | tttataaggaaaagcgatgagcttctgcacaacgtgaatgccggtaaatccacc |
| | | | | | | acaaactag |
| Codon | AA | 100 | — | — | 7 | See above |
| Optimized | | | | | | |
| Soluble | | | | | | |
| High GC | NA | 77 | 58 | 100 | 5 | atggagttgctcatcctcaaggccaacgccatcaccacgatcctcacggccgtc |
| (GC rich) | | | | | | acgttctgcttcgcgtccggccagaacatcaccgaggagttctaccagtcgacg |
| soluble | | | | | | tgcagcgccgtgagcaagggctacctcagcgcgctgaggacgggctggtacacc |
| | | | | | | agcgtcatcacgatcgagttgagcaacatcaagaagaacaagtgcaacggcacc |
| | | | | | | gacgcgaaggtcaagttgatcaagcaggagttggacaagtacaagaacgccgtg |
| | | | | | | accgagttgcagttgctcatgcagagcacgccggcgacgaacaaccgcgccagg |
| | | | | | | agggagctcccgaggttcatgaactacacgctcaacaacgccaagaagaccaac |
| | | | | | | gtgaccttgagcaagaagaggaagaggaggttcctcggcttcttgttgggcgtc |
| | | | | | | ggctcggccatcgccagcggcgtggccgtctcgaaggtcctgcacctggagggc |
| | | | | | | gaggtgaacaagatcaagagcgcgctgctctccacgaacaaggccgtcgtcagc |
| | | | | | | ttgtccaacggcgtcagcgtcttgaccagcaaggtgttggacctcaagaactac |
| | | | | | | atcgacaagcagttgttgccgatcgtgaacaagcagagctgcagcatctcgaac |
| | | | | | | atcgagaccgtgatcgagttccagcagaagaacaacaggctgctcgagatcacc |
| | | | | | | agggagttcagcgtcaacgccggcgtcacgacgccggtcagcacctacatgttg |
| | | | | | | accaacagcgagttgttgtccttgatcaacgacatgccgatcaccaacgaccag |
| | | | | | | aagaagttgatgtccaacaacgtgcagatcgtcaggcagcagagctactcgatc |
| | | | | | | atgtccatcatcaaggaggaggtcttggcctacgtcgtgcagttgccgctgtac |
| | | | | | | ggcgtcatcgacacgccctgctggaagctgcacacgtccccgctgtgcacgacc |
| | | | | | | aacacgaaggaggggtccaacatctgcttgaccaggaccgacaggggctggtac |
| | | | | | | tgcgacaacgccggctccgtgtcgttcttcccgcaggccgagacctgcaaggtc |
| | | | | | | cagtccaaccgcgtcttctgcgacacgatgaacagcttgacgttgccgagcgag |
| | | | | | | gtcaacctctgcaacgtcgacatcttcaaccccaagtacgactgcaagatcatg |
| | | | | | | acgtccaagaccgacgtcagcagctccgtgatcacgtcgctcggcgccatcgtg |
| | | | | | | tcctgctacggcaagaccaagtgcaccgcgtccaacaagaaccgcggcatcatc |
| | | | | | | aagacgttctcgaacgggtgcgactacgtctcgaacaagggggtggacaccgtg |
| | | | | | | tccgtcggcaacacgttgtactacgtcaacaagcaggagggcaagagcctctac |
| | | | | | | gtcaagggcgagccgatcatcaacttctacgacccgttggtcttcccctcggac |
| | | | | | | gagttcgacgcgtcgatctcgcaggtcaacgagaagatcaaccagagcctggcg |
| | | | | | | ttcatccggaagtccgacgagttgttgcacaacgtgaacgccggcaagtccacc |
| | | | | | | acgaactaa |
| High GC | AA | 100 | — | — | 7 | See above |
| (GC rich) | | | | | | |
| soluble | | | | | | |
| Med GC | NA | 85 | 51 | 76 | 6 | atggagttgctcatcctcaaggccaacgccatcaccacgatcctcacggcagtc |
| (HL2) | | | | | | acattctgtttcgcttctggtcagaacatcactgaggaattctaccaatcgacg |
| soluble | | | | | | tgcagtgcagttagcaagggctatctcagtgctctgagaacgggttggtatacc |
| | | | | | | agtgtcatcactatcgagttgagtaacatcaagaagaacaagtgtaacggaacc |
| | | | | | | gatgcgaaggtaaagttgatcaagcaggagttggacaagtacaagaacgctgta |
| | | | | | | acagagttgcagttgctcatgcagagcacaccagcgacgaacaaccgagccagg |
| | | | | | | agagagctaccaaggttcatgaactacacgctcaacaacgccaagaagaccaac |
| | | | | | | gtgacattgagcaagaagaggaagaggagattcctcggtttcttgttgggtgtc |
| | | | | | | ggatctgcaatcgccagtggcgttgctgtctcgaaggtcctgcacctagaaggg |
| | | | | | | gaagtgaacaagatcaagagtgctctgctatccacgaacaaggctgtcgtcagc |
| | | | | | | ttgtcaaacggagtcagtgtcttgaccagcaaggtgttggacctcaagaactac |
| | | | | | | atcgacaagcagttgttacctatcgtgaacaagcaaagctgcagcatctcaaac |
| | | | | | | atcgagactgtgatcgagttccagcagaagaacaacagactactagagatcacc |
| | | | | | | agggagttcagtgtcaacgcaggtgtaacgacacctgtcagcacttacatgttg |
| | | | | | | actaacagtgagttgttgtcattgatcaacgacatgcctatcaccaacgatcag |
| | | | | | | aagaagttgatgtccaacaacgtgcagatcgtcagacagcagagctactcgatc |
| | | | | | | atgtccatcatcaaggaggaagtcttggcatacgtagtacagttgccactgtat |
| | | | | | | ggtgtcatcgacacaccctgctggaagctgcacacgtcccctctatgtacgacc |
| | | | | | | aacacgaaggaagggtccaacatctgcttgaccaggactgacagaggatggtac |
| | | | | | | tgcgacaacgcaggatccgtgtcgttcttcccacaggctgagacctgcaaggtc |
| | | | | | | cagtccaaccgagtcttctgcgacacgatgaacagcttgacgttgccgagtgag |
| | | | | | | gtaaacctctgcaacgtcgacatcttcaaccccaagtacgactgcaagatcatg |
| | | | | | | acgtccaagaccgatgtcagcagctccgtgatcacatcgctcggagccatcgtg |
| | | | | | | tcatgctacggcaagaccaagtgcacagcgtccaacaagaaccgtggaatcatc |
| | | | | | | aagacgttctcgaacgggtgcgactacgtctcaaacaagggggtggacactgtg |
| | | | | | | tctgtaggcaacacattgtactacgtaaacaagcaggaaggtaagagcctctac |
| | | | | | | gtcaagggtgaaccaatcatcaacttctacgacccgttggtcttcccctctgac |
| | | | | | | gagttcgacgcatcgatctctcaggtcaacgagaagatcaaccagagcctagca |
| | | | | | | ttcatccggaagtccgacgagttgttgcacaacgtgaatgccggtaagtccacc |
| | | | | | | acaaactaa |
| Med GC | AA | 100 | — | — | 7 | See above |
| (HL2) | | | | | | |
| soluble | | | | | | |

hPIV3/Texas/12084/1983

| | | | | | | |
|---|---|---|---|---|---|---|
| WT | NA | — | 35 | 29 | 8 | atgccaacttcaatactgctaattattacaaccatgatcatggcatctttctgc |
| | | | | | | caaatagatatcacaaaactacagcatgtaggtgtattggtcaatagtcccaaa |
| | | | | | | ggaatgaagatatcacaaaactttgaaacaagatatctgattttgagcctcata |
| | | | | | | ccaaaaatagaagactctaactcttgtggtgaccaacagatcaagcaatacaag |
| | | | | | | aagctattggatagactgatcatccctttatatgatggattaagattacagaaa |
| | | | | | | gatgtgatagtaaccaatcaagaatccaatgaaaacactgatcccagaacaaaa |
| | | | | | | cgattctttggaggggtaattggaactattgctctgggagtagcaacctcagca |
| | | | | | | caaattacagcggcagttgctttggttgaagccaagcaggcaagatcagacatc |
| | | | | | | gaaaaactcaaagaagcaattagggacacaaataaagcagtgcagtcagttcag |
| | | | | | | agctccataggaaatctaatagtagcaattaaatcagtccaggattatgttaac |
| | | | | | | aaagaaatcgtgccatcgattgcgaggctaggttgtgaagcagcaggacttcaa |
| | | | | | | ttaggaattgcattaacacagcattactcagaattaacaaacatatttggtgat |
| | | | | | | aacataggatcgttacaagaaaaaggaataaaattacaaggtatagcatcatta |
| | | | | | | taccgcacaaatatcacagaaatatttacaacatcaacagttgataaatatgat |
| | | | | | | atttatgatctgttatttacagaatcaataaaggtgagagttatagatgttgac |
| | | | | | | ttgaatgattactcaatcaccctccaagtcagactccctttattaactaggctg |
| | | | | | | ctgaacactcagatctacaaagtagattccatatcatataacattcaaaacaga |
| | | | | | | gaatggtatatccctcttcccagccatatcatgacaaaaggggcatttctaggt |
| | | | | | | ggagcagatgtcaaagaatgtatagaagcattcagcagctatatatgcccttct |
| | | | | | | gatccaggatttgtattaaaccatgaaatagagagctgcttatcaggaaacata |
| | | | | | | tctcaatgtccaagaaccacagtcacatcagacattgttccaagatatgcattt |
| | | | | | | gtcaatggaggagtggttgcaaactgtataacaaccacttgtacatgcaacgga |
| | | | | | | atcggtaatagaatcaatcaaccacctgatcaaggaataaaaattataacacat |
| | | | | | | aaagaatgtagtacaataggtatcaacggaatgctgttcaatacaaataaagaa |
| | | | | | | ggaactcttgcattctacacaccaaatgatataacactaaacaattctgttgca |
| | | | | | | cttgatccaattgacatatcaatcgagctcaacaaggccaaatcagatctagaa |
| | | | | | | gaatcaaaagaatggataagaaggtcaaatcaaaaactagattccattggaaat |
| | | | | | | tggcatcaatctagcactacagtcataattattttgataatgatcattatattg |
| | | | | | | tttataatta |
| WT | AA | — | — | — | 9 | mptsilliittmimasfcqiditklqhvgvlvnspkgmkisqnfetrylilsli |
| | | | | | | pkiedsnscgdqqikqykklldrliiplydglrlqkdvivtnqesnentdprtk |
| | | | | | | rffggvigtialgvatsaqitaavalveakqarsdieklkeairdtnkavqsvq |
| | | | | | | ssignlivaiksvqdyvnkeivpsiarlgceaaglqlgialtqhyseltnifgd |
| | | | | | | nigslqekgiklqgiaslyrtniteifttstvdkydiydllftesikvrvidvd |
| | | | | | | lndysitlqvrlplltrllntqiykvdsisyniqnrewyiplpshimtkgaflg |
| | | | | | | gadvkecieafssyicpsdpgfvlnheiesclsgnisqcprttvtsdivpryaf |
| | | | | | | vnggvvancitttctcngignrinqppdqgikiithkecstigingmlfntnke |
| | | | | | | gtlafytpnditlnnsvaldpidisielnkaksdleeskewirrsnqkldsign |
| | | | | | | whqssttviiilimiiilfiinvtiitiaikyyriqkrnrvdqndkpyvltnk |
| Soluble | NA | 99 | 36 | 29 | 10 | atgccaacttcaatactgctaattattacaaccatgatcatggcatctttctgc |
| (hTexFsol) | | | | | | caaatagatatcacaaaactacagcatgtaggtgtattggtcaatagtcccaaa |
| | | | | | | ggaatgaagatatcacaaaactttgaaacaagatatctgattttgagcctcata |
| | | | | | | ccaaaaatagaagactctaactcttgtggtgaccaacagatcaagcaatacaag |
| | | | | | | aagctattggatagactgatcatccctttatatgatggattaagattacagaaa |
| | | | | | | gatgtgatagtaaccaatcaagaatccaatgaaaacactgatcccagaacaaaa |
| | | | | | | cgattctttggaggggtaattggaactattgctctgggagtagcaacctcagca |
| | | | | | | caaattacagcggcagttgctttggttgaagccaagcaggcaagatcagacatc |
| | | | | | | gaaaaactcaaagaagcaattagggacacaaataaagcagtgcagtcagttcag |
| | | | | | | agctccataggaaatctaatagtagcaattaaatcagtccaggattatgttaac |
| | | | | | | aaagaaatcgtgccatcgattgcgaggctaggttgtgaagcagcaggacttcaa |
| | | | | | | ttaggaattgcattaacacagcattactcagaattaacaaacatatttggtgat |
| | | | | | | aacataggatcgttacaagaaaaaggaataaaattacaaggtatagcatcatta |
| | | | | | | taccgcacaaatatcacagaaatatttacaacatcaacagttgataaatatgat |
| | | | | | | atttatgatctgttatttacagaatcaataaaggtgagagttatagatgttgac |
| | | | | | | ttgaatgattactcaatcaccctccaagtcagactccctttattaactaggctg |
| | | | | | | ctgaacactcagatctacaaagtagattccatatcatataacattcaaaacaga |
| | | | | | | gaatggtatatccctcttcccagccatatcatgacaaaaggggcatttctaggt |
| | | | | | | ggagcagatgtcaaagaatgtatagaagcattcagcagctatatatgcccttct |
| | | | | | | gatccaggatttgtattaaaccatgaaatagagagctgcttatcaggaaacata |
| | | | | | | tctcaatgtccaagaaccacagtcacatcagacattgttccaagatatgcattt |
| | | | | | | gtcaatggaggagtggttgcaaactgtataacaaccacttgtacatgcaacgga |
| | | | | | | atcggtaatagaatcaatcaaccacctgatcaaggaataaaaattataacacat |
| | | | | | | aaagaatgtagtacaataggtatcaacggaatgctgttcaatacaaataaagaa |
| | | | | | | ggaactcttgcattctacacaccaaatgatataacactaaacaattctgttgca |
| | | | | | | cttgatccaattgacatatcaatcgagctcaacaaggccaaatcagatctagaa |
| | | | | | | gaatcaaaagaatggataagaaggtcaaatcaaaaactagattccattggaaat |
| | | | | | | tggcattaa |
| Soluble | AA | 100 | — | — | 12 | mptsilliittmimasfcqiditklqhvgylvnspkgmkisqnfetrylilsli |
| (hTexFsol) | | | | | | pkiedsnscgdqqikqykklldrliiplydglrlqkdvivtnqesnentdprtk |
| | | | | | | rffggvigtialgvatsaqitaavalveakqarsdieklkeairdtnkavqsvq |
| | | | | | | ssignlivaiksvqdyvnkeivpsiarlgceaaglqlgialtqhyseltnifgd |
| | | | | | | nigslqekgiklqgiaslyrtniteifttstvdkydiydllftesikvrvidvd |
| | | | | | | lndysitlqvrlplltrllntqiykvdsisyniqnrewyiplpshimtkgaflg |
| | | | | | | gadvkecieafssyicpsdpgfvlnheiesclsgnisqcprttvtsdivpryaf |
| | | | | | | vnggvvancitttctcngignrinqppdqgikiithkecstigingmlfntnke |
| | | | | | | gtlafytpnditlnnsvaldpidisielnkaksdleeskewirrsnqkldsign |
| | | | | | | wh |
| High GC | NA | 77 | 60 | 100 | 11 | atgccgacgtccatcctgctgatcatcacgaccatgatcatggcgtcgttctgc |
| (HL1sol) | | | | | | cagatcgacatcacgaagctccagcacgtcggcgtcttggtcaacagccccaag |
| | | | | | | ggcatgaagatctcgcagaacttcgagaccaggtacctgatcttgagcctcatc |
| | | | | | | ccgaagatcgaggactcgaactcctgcggcgaccagcagatcaagcagtacaag |
| | | | | | | aagctcttggacaggctgatcatcccgttgtacgacggcttgaggttgcagaag |
| | | | | | | gacgtgatcgtcaccaaccaggagtccaacgagaacaccgaccccaggacgaag |
| | | | | | | cgcttcttcggcggggtcatcggcacgatcgcgctgggggtcgccacctcggcc |
| | | | | | | cagatcaccgcggcggtcgcgttggtcgaggccaagcaggcgaggtccgacatc |
| | | | | | | gagaagctcaaggaggccatcagggacacgaacaaggccgtgcagtccgtccag |
| | | | | | | agctccatcggcaacctgatcgtcgcgatcaagtccgtccaggactacgtgaac |
| | | | | | | aaggagatcgtgccgtcgatcgcgaggctcggctgcgaggccgccggcctgcag |
| | | | | | | ttgggcatcgcgttgacgcagcactactcggagttgaccaacatcttcggcgac |
| | | | | | | aacatcggctcgttgcaggagaagggcatcaagttgcagggcatcgcgtccttg |
| | | | | | | taccgcacgaacatcacggagatcttcacgacctcgaccgtcgacaagtacgac |
| | | | | | | atctacgacctgttgttcacggagtcgatcaaggtgagggtcatcgacgtggac |
| | | | | | | ttgaacgactactcgatcaccctccaggtcaggctccccttgttgaccaggctg |
| | | | | | | ctgaacacgcagatctacaaggtcgactccatctcgtacaacatccagaacagg |
| | | | | | | gagtggtacatcccgctgcccagccacatcatgaccaagggggccttcctcggc |
| | | | | | | ggcgccgacgtcaaggagtgcatcgaggcgttcagcagctacatctgcccgtcg |
| | | | | | | gaccccggcttcgtgttgaaccacgagatcgagagctgcttgtcgggcaacatc |
| | | | | | | tcgcagtgcccgaggaccacggtcacgtccgacatcgtgccgaggtacgccttc |
| | | | | | | gtcaacggcggcgtggtcgcgaactgcatcacgaccacgtgcacgtgcaacggc |
| | | | | | | atcggcaacaggatcaaccagccgccggaccagggcatcaagatcatcacgcac |
| | | | | | | aaggagtgcagcaccatcggcatcaacgggatgctgttcaacacgaacaaggag |
| | | | | | | ggcacgctggcgttctacacgccgaacgacatcacgctgaacaactcggtcgcg |
| | | | | | | ctcgacccgatcgacatctcgatcgagctcaacaaggccaagtcggacctcgag |
| | | | | | | gagtccaaggagtggatcaggaggtcgaaccagaagctcgactccatcggcaac |
| | | | | | | tggcactaa |
| High GC | AA | 100 | — | — | 12 | See above |
| (HL1sol) | | | | | | |
| Soluble | NA | 100 | 36 | 29 | 13 | atgccaacttcaatactgctaattattacaaccatgatcatggcatctttctgc |
| with 6xhis | | | | | | caaatagatatcacaaaactacagcatgtaggtgtattggtcaatagtcccaaa |
| tag | | | | | | ggaatgaagatatcacaaaactttgaaacaagatatctgattttgagcctcata |
| (hTexFsolhis) | | | | | | ccaaaaatagaagactctaactcttgtggtgaccaacagatcaagcaatacaag |
| | | | | | | aagctattggatagactgatcatccctttatatgatggattaagattacagaaa |
| | | | | | | gatgtgatagtaaccaatcaagaatccaatgaaaacactgatcccagaacaaaa |
| | | | | | | cgattctttggaggggtaattggaactattgctctgggagtagcaacctcagca |
| | | | | | | caaattacagcggcagttgctttggttgaagccaagcaggcaagatcagacatc |
| | | | | | | gaaaaactcaaagaagcaattagggacacaaataaagcagtgcagtcagttcag |
| | | | | | | agctccataggaaatctaatagtagcaattaaatcagtccaggattatgttaac |
| | | | | | | aaagaaatcgtgccatcgattgcgaggctaggttgtgaagcagcaggacttcaa |
| | | | | | | ttaggaattgcattaacacagcattactcagaattaacaaacatatttggtgat |
| | | | | | | aacataggatcgttacaagaaaaaggaataaaattacaaggtatagcatcatta |
| | | | | | | taccgcacaaatatcacagaaatatttacaacatcaacagttgataaatatgat |
| | | | | | | atttatgatctgttatttacagaatcaataaaggtgagagttatagatgttgac |
| | | | | | | ttgaatgattactcaatcaccctccaagtcagactccctttattaactaggctg |
| | | | | | | ctgaacactcagatctacaaagtagattccatatcatataacattcaaaacaga |
| | | | | | | gaatggtatatccctcttcccagccatatcatgacaaaaggggcatttctaggt |
| | | | | | | ggagcagatgtcaaagaatgtatagaagcattcagcagctatatatgcccttct |
| | | | | | | gatccaggatttgtattaaaccatgaaatagagagctgcttatcaggaaacata |
| | | | | | | tctcaatgtccaagaaccacagtcacatcagacattgttccaagatatgcattt |
| | | | | | | gtcaatggaggagtggttgcaaactgtataacaaccacttgtacatgcaacgga |
| | | | | | | atcggtaatagaatcaatcaaccacctgatcaaggaataaaaattataacacat |
| | | | | | | aaagaatgtagtacaataggtatcaacggaatgctgttcaatacaaataaagaa |
| | | | | | | ggaactcttgcattctacacaccaaatgatataacactaaacaattctgttgca |
| | | | | | | cttgatccaattgacatatcaatcgagctcaacaaggccaaatcagatctagaa |
| | | | | | | gaatcaaaagaatggataagaaggtcaaatcaaaaactagattccattggaaat |
| | | | | | | tggcatcaccaccatcaccatcactaa |
| Soluble | AA | 100 | — | — | 15 | mptsilliittmimasfcqiditklqhvgvlvnspkgmkisqnfetrylilsli |
| with 6xhis | | | | | | pkiedsnscgdqqikqykklldrliiplydglrlqkdvivtnqesnentdprtk |
| tag | | | | | | rffggvigtialgvatsaqitaavalveakqarsdieklkeairdtnkavqsvg |
| (hTexFsolhis) | | | | | | ssignlivaiksvqdyvnkeivpsiarlgceaaglqlgialtqhyseltnifgd |
| | | | | | | nigslqekgiklqgiaslyrtniteifttstvdkydiydllftesikvrvidvd |
| | | | | | | lndysitlqvrlplltrllntqiykvdsisyniqnrewyiplpshimtkgaflg |
| | | | | | | gadvkecieafssyicpsdpgfvlnheiesclsgnisqcprttvtsdivpryaf |
| | | | | | | vnggvvancitttctcngignrinqppdggikiithkecstigingmlfntnke |
| | | | | | | gtlafytpnditlnnsvaldpidisielnkaksdleeskewirrsnqkldsign |
| | | | | | | whhhhhhh |
| hi GC with | NA | 77 | 60 | 100 | 14 | atgccgacgtccatcctgctgatcatcacgaccatgatcatggcgtcgttctgc |
| 6xhis tag | | | | | | cagatcgacatcacgaagctccagcacgtcggcgtcttggtcaacagccccaag |
| (HL1solhis) | | | | | | ggcatgaagatctcgcagaacttcgagaccaggtacctgatcttgagcctcatc |
| | | | | | | ccgaagatcgaggactcgaactcctgcggcgaccagcagatcaagcagtacaag |
| | | | | | | aagctcttggacaggctgatcatcccgttgtacgacggcttgaggttgcagaag |
| | | | | | | gacgtgatcgtcaccaaccaggagtccaacgagaacaccgaccccaggacgaag |
| | | | | | | cgcttcttcggcggggtcatcggcacgatcgcgctgggggtcgccacctcggcc |
| | | | | | | cagatcaccgcggcggtcgcgttggtcgaggccaagcaggcgaggtccgacatc |
| | | | | | | gagaagctcaaggaggccatcagggacacgaacaaggccgtgcagtccgtccag |
| | | | | | | agctccatcggcaacctgatcgtcgcgatcaagtccgtccaggactacgtgaac |
| | | | | | | aaggagatcgtgccgtcgatcgcgaggctcggctgcgaggccgccggcctgcag |
| | | | | | | ttgggcatcgcgttgacgcagcactactcggagttgaccaacatcttcggcgac |
| | | | | | | aacatcggctcgttgcaggagaagggcatcaagttgcagggcatcgcgtccttg |
| | | | | | | taccgcacgaacatcacggagatcttcacgacctcgaccgtcgacaagtacgac |
| | | | | | | atctacgacctgttgttcacggagtcgatcaaggtgagggtcatcgacgtggac |
| | | | | | | ttgaacgactactcgatcaccctccaggtcaggctccccttgttgaccaggctg |
| | | | | | | ctgaacacgcagatctacaaggtcgactccatctcgtacaacatccagaacagg |
| | | | | | | gagtggtacatcccgctgcccagccacatcatgaccaagggggccttcctcggc |
| | | | | | | ggcgccgacgtcaaggagtgcatcgaggcgttcagcagctacatctgcccgtcg |
| | | | | | | gaccccggcttcgtgttgaaccacgagatcgagagctgcttgtcgggcaacatc |
| | | | | | | tcgcagtgcccgaggaccacggtcacgtccgacatcgtgccgaggtacgccttc |
| | | | | | | gtcaacggcggcgtggtcgcgaactgcatcacgaccacgtgcacgtgcaacggc |
| | | | | | | atcggcaacaggatcaaccagccgccggaccagggcatcaagatcatcacgcac |
| | | | | | | aaggagtgcagcaccatcggcatcaacgggatgctgttcaacacgaacaaggag |
| | | | | | | ggcacgctggcgttctacacgccgaacgacatcacgctgaacaactcggtcgcg |
| | | | | | | ctcgacccgatcgacatctcgatcgagctcaacaaggccaagtcggacctcgag |
| | | | | | | gagtccaaggagtggatcaggaggtcgaaccagaagctcgactccatcggcaac |
| | | | | | | tggcaccaccaccatcaccatcactaa |
| High GC | AA | 100 | — | — | 15 | See above |
| with 6xhis | | | | | | |
| tag | | | | | | |
| (HL1solhis) | | | | | | |

### Example 7

### Examples of Embodiments

Provided hereafter are non-limiting examples of certain embodiments of the technology.

A1. An isolated nucleic acid comprising a nucleotide sequence having a GC content of about 51% or greater that encodes a soluble viral fusion protein comprising an amino acid sequence 90% or more identical to SEQ ID NO: 7.

A2. The isolated nucleic acid of embodiment A1, wherein the nucleotide sequence encodes a protein comprising an amino acid sequence 95% or more identical to SEQ ID NO: 7.

A3. The isolated nucleic acid of embodiment A1, wherein the nucleotide sequence encodes a protein comprising an amino acid sequence of SEQ ID NO: 7.

A4. The isolated nucleic acid of any one of embodiments A1 to A3, wherein the soluble viral fusion protein lacks a functional membrane association region.

A5. The isolated nucleic acid of embodiment A4, wherein the soluble viral fusion protein lacks C-terminal transmembrane region amino acids corresponding to amino acids 525 to 574 of SEQ ID NO: 2.

A6. The isolated nucleic acid of any one of embodiments A1 to A5, wherein the protein comprises a tag.

A6.1 The isolated nucleic acid of any one of embodiments A1 to A5, wherein the protein comprises no tag.

A7. The isolated nucleic acid of any one of embodiments A1 to A6.1, wherein the GC3 content of the nucleotide sequence is 76% or greater.

A8. The isolated nucleic acid of embodiment A7, comprising the nucleotide sequence of SEQ ID NO: 6.

A9. The isolated nucleic acid of any one of embodiments A1 to A8, wherein the GC content of the nucleotide sequence is about 58% or greater.

A10. The isolated nucleic acid of embodiment A9, wherein the GC3 content of the nucleotide sequence is about 100%.

A11. The isolated nucleic acid of embodiment A9 or A10, comprising the nucleotide sequence of SEQ ID NO: 5.

A12. The isolated nucleic acid of any one of embodiments A1 to A11, wherein the nucleotide sequence is 65% or more identical to SEQ ID NO: 3.

A13. The isolated nucleic acid of embodiment A12, wherein the nucleotide sequence is 73% or more identical to SEQ ID NO: 3.

A14. The isolated nucleic acid of embodiment A13, wherein the nucleotide sequence is 77% or more identical to SEQ ID NO: 3.

A15. The isolated nucleic acid of any one of embodiments A1 to A14, further comprising a cis-regulatory element in functional association with the nucleotide sequence.

A16. The isolated nucleic acid of embodiment A15, wherein the cis-regulatory element comprises a post transcriptional processing element.

A17. The isolated nucleic acid of embodiment A16, wherein the post transcriptional regulatory element is from woodchuck hepatitis virus.

A18. The isolated nucleic acid of any one of embodiments A1 to A17, which is in an expression vector.

B1. A cell comprising the isolated nucleic acid of embodiment A18.

B2. A cell comprising the nucleotide sequence of any one of embodiments A1 to A14 integrated into cellular DNA.

B3. The cell of embodiment B1 or B2 that expresses at least 2 micrograms of the protein per milliliter of cells.

B4. The cell of embodiment B3, that expresses at least 6 micrograms of the protein per milliliter of cells.

B5. The cell of embodiment B4, that expresses at least 200 micrograms of the protein per milliliter of cells.

B6. The cell of embodiment B5, that expresses at least 500 micrograms of the protein per milliliter of cells.

B7. The cell of embodiment B6, that expresses at least 1 milligram of the protein per milliliter of cells.

B8. The cell of embodiment B7, that expresses at least 1.3 milligram of the protein per milliliter of cells.

B9. The cell of embodiment B8, that expresses at least 1.6 milligram of the protein per milliliter of cells.

B10. The cell of any one of embodiments B1 to B9, which secretes the soluble viral fusion protein.

B11. The cell of any one of embodiments B1 to B10, which is a mammalian cell.

B12. The cell of embodiment B11, wherein the cell is a non-adherent cell.

B13. The cell of embodiment B11 or B12, wherein the cell is a CHO cell or CHO-derived cell.

B14. The cell of embodiment B13, wherein the cell is a CAT-S cell.

B15. The cell of embodiment B13, wherein the cell is a CHO-S cell.

B16. The cell of embodiment B11, wherein the cell is a Vero cell.

B17. The cell of embodiment B11, wherein the cell is a MRC-5 cell.

B18. The cell of embodiment B11, wherein the cell is a BSR-T7 cell.

B19. The cell of any one of embodiments B1 to B18, wherein the cell synthesizes nucleic acid encoding the viral fusion protein in the nucleus.

B20. The cell of any one of embodiments B1 to B19, which further comprises the cis-regulatory element of any one of embodiments A15 to A17 in functional association with the nucleotide sequence.

C1. A method for expressing a soluble viral fusion protein, comprising contacting a plurality of cells comprising the nucleotide sequence of any one of embodiments A1 to A14 to conditions under which the protein is produced.

C2. The method of embodiment C1, wherein the nucleotide sequence is in an expression vector in the cells.

C3. The method of embodiment C1, wherein the nucleotide sequence is in cellular DNA of the cells.

C4. The method of any one of embodiments C1 to C3, wherein the cells are mammalian cells.

C5. The method of embodiment C4, wherein the cells are non-adherent cells.

C6. The method of embodiment C5, wherein the cells are CHO cells or CHO-derived cells.

C7. The method of embodiment C6, wherein the cells are CAT-S cells.

C8. The method of embodiment C6, wherein the cells are CHO-S cells.

C9. The method of embodiment C4 wherein the cells are Vero cells.

C10. The method of embodiment C4 wherein the cells are MRC-5 cells.

C11. The method of embodiment C4 wherein the cells are BSR-T7 cells.

C12. The method of any one of embodiments C1 to 011, wherein at least 2 micrograms of the protein per milliliter of cells is produced.

C13. The method of embodiment C12, wherein at least 6 micrograms of the protein per milliliter of cells is produced.

C14. The method of embodiment C13, wherein at least 200 micrograms of the protein per milliliter of cells is produced.

C15. The method of embodiment C14, wherein at least 500 micrograms of the protein per milliliter of cells is produced.

C16. The method of embodiment C15, wherein at least 1 milligram of the protein per milliliter of cells is produced.

C17. The method of embodiment C16, wherein at least 1.3 milligram of the protein per milliliter of cells is produced.

C18. The method of embodiment C17, wherein at least 1.6 milligram of the protein per milliliter of cells is produced.

C19. The method of any one of embodiments C1 to C18, wherein the cells secrete the protein.

C20. The method of any one of embodiments C1 to C19, wherein the protein is produced for 7 or more days.

C21. The method of any one of embodiments C1 to C20, wherein the cells are cultured under animal product-free culture conditions.

C22. The method of any one of embodiments C1 to C21, further comprising determining the amount of protein produced by the cells.

C23. The method of any one of embodiments C1 to C22, further comprising isolating the protein.

C24. The method of any one of embodiments C1 to C23, wherein the cell synthesizes nucleic acid encoding the viral fusion protein in the nucleus.

C25. The method of any one of embodiments C1 to C24, wherein the cell further comprises the cis-regulatory element of any one of embodiments A15 to A17 in functional association with the nucleotide sequence of any one of embodiments A1 to A14.

C26. The method of any one of embodiments C1 to C25, which comprises introducing into the cell nucleus the nucleotide sequence of any one of embodiments A1 to A14.

C27. The method of embodiment C26, which comprises introducing into the cell nucleus the cis-regulatory element of any one of embodiments A15 to A17 in functional association with the nucleotide sequence of any one of embodiments A1 to A14.

C28. The method of embodiment C26 or C27, wherein the introducing into the cell nucleus is by nucleotransfection.

D1. A nucleic acid comprising a nucleotide sequence (i) having a GC content of about 51% or greater, (ii) that is 73% or more identical to SEQ ID NO: 1, and (iii) that encodes a viral fusion protein comprising an amino acid sequence 90% or more identical to SEQ ID NO: 2.

D2. The isolated nucleic acid of embodiment D1, wherein the nucleotide sequence encodes a protein comprising an amino acid sequence 95% or more identical to SEQ ID NO: 2.

D3. The isolated nucleic acid of embodiment D1, wherein the nucleotide sequence encodes a protein comprising an amino acid sequence of SEQ ID NO: 2.

D4. The isolated nucleic acid of any one of embodiments D1 to D3, wherein the protein comprises a tag.

D4.1 The isolated nucleic acid of any one of embodiments D1 to D3, wherein the protein comprises no tag.

D5. The isolated nucleic acid of any one of embodiments D1 to D4.1, wherein the GC3 content of the nucleotide sequence is 76% or greater.

D6. The isolated nucleic acid of any one of embodiments D1 to D5, wherein the GC content of the nucleotide sequence is about 58% or greater.

D7. The isolated nucleic acid of embodiment D6, wherein the GC3 content of the nucleotide sequence is about 100%.

D8. The isolated nucleic acid of embodiment D6 or D7, comprising the nucleotide sequence of SEQ ID NO: 17.

D9. The isolated nucleic acid of embodiment D8, wherein the nucleotide sequence is 77% or more identical to SEQ ID NO: 1.

D10. The isolated nucleic acid of any one of embodiments D1 to D9, further comprising a cis-regulatory element in functional association with the nucleotide sequence.

D11. The isolated nucleic acid of embodiment D10, wherein the cis-regulatory element comprises a post transcriptional processing element.

D12. The isolated nucleic acid of embodiment D11, wherein the post transcriptional regulatory element is from woodchuck hepatitis virus.

D13. The isolated nucleic acid of any one of embodiments D1 to D12, which is in an expression vector.

E1. A cell comprising the isolated nucleic acid of embodiment D13.

E2. A cell comprising the nucleotide sequence of any one of embodiments D1 to D9 integrated into cellular DNA.

E3. The cell of any one of embodiments E1 to E2, wherein the protein is retained in the cell membrane.

E4. The cell of any one of embodiments E1 to E2, which secretes the viral fusion protein.

E5. The cell of any one of embodiments E1 to E4, which is a mammalian cell.

E6. The cell of embodiment E5, wherein the cell is a non-adherent cell.

E7. The cell of embodiment E5 or E6, wherein the cell is a CHO cell or CHO-derived cell.

E8. The cell of embodiment E7, wherein the cell is a CAT-S cell.

E9. The cell of embodiment E7, wherein the cell is a CHO-S cell.

E10. The cell of embodiment E5, wherein the cell is a Vero cell.

E11. The cell of embodiment E5, wherein the cell is a MRC-5 cell.

E12. The cell of embodiment E5, wherein the cell is a BSR-T7 cell.

E13. The cell of any one of embodiments E1 to E12, wherein the cell synthesizes nucleic acid encoding the viral fusion protein in the nucleus.

E14. The cell of any one of embodiments E1 to E13, which further comprises the cis-regulatory element of any one of embodiments D10 to D12 in functional association with the nucleotide sequence.

F1. A method for expressing a viral fusion protein, comprising contacting a plurality of cells comprising the nucleotide sequence of any one of embodiments D1 to D9 to conditions under which the protein is produced.

F2. The method of embodiment F1, wherein the nucleotide sequence is in an expression vector in the cells.

F3. The method of embodiment F1, wherein the nucleotide sequence is in cellular DNA of the cells.

F4. The method of any one of embodiments F1 to F3, wherein the cells are mammalian cells.

F5. The method of embodiment F4, wherein the cells are non-adherent cells.

F6. The method of embodiment F5, wherein the cells are CHO cells or CHO-derived cells.

F7. The method of embodiment F6, wherein the cells are CAT-S cells.

F8. The method of embodiment F6, wherein the cells are CHO-S cells.

F9. The method of embodiment F4 wherein the cells are Vero cells.

F10. The method of embodiment F4 wherein the cells are MRC-5 cells.

F11. The method of embodiment F4 wherein the cells are BSR-T7 cells.

F12. The method of any one of embodiments F1 to F11, wherein the protein is retained in the cell membrane.

F13. The method of any one of embodiments F1 to F11, wherein the cells secrete the protein.

F14. The method of any one of embodiments F1 to F13, wherein the protein is produced for 7 or more days.

F15. The method of any one of embodiments F1 to F14, wherein the cells are cultured under animal product-free culture conditions.

F16. The method of any one of embodiments F1 to F15, further comprising determining the amount of protein produced by the cells.

F17. The method of any one of embodiments F1 to F16, further comprising isolating the protein.

F18. The method of any one of embodiments F1 to F17, wherein the cell synthesizes nucleic acid encoding the viral fusion protein in the nucleus.

F19. The method of any one of embodiments F1 to F18, wherein the cell further comprises the cis-regulatory element of any one of embodiments D10 to D12 in functional association with the nucleotide sequence of any one of embodiments D1 to D9.

F20. The method of any one of embodiments F1 to F19, which comprises introducing into the cell nucleus the nucleotide sequence of any one of embodiments D1 to D9.

F21. The method of embodiment F20, which comprises introducing into the cell nucleus the cis-regulatory element of any one of embodiments D10 to D12 in functional association with the nucleotide sequence of any one of embodiments D1 to D9.

F22. The method of embodiment F20 or F21, wherein the introducing into the cell nucleus is by nucleotransfection.

G1. A nucleic acid comprising a nucleotide sequence having a GC content of about 51% or greater that encodes a viral fusion protein comprising an amino acid sequence 90% or more identical to SEQ ID NO: 12.

G2. The isolated nucleic acid of embodiment G1, wherein the nucleotide sequence encodes a protein comprising an amino acid sequence 95% or more identical to SEQ ID NO: 12.

G3. The isolated nucleic acid of embodiment G1, wherein the nucleotide sequence encodes a protein comprising an amino acid sequence of SEQ ID NO: 12.

G4. The isolated nucleic acid of any one of embodiments G1 to G3, wherein the protein comprises a tag.

G4.1 The isolated nucleic acid of any one of embodiments G1 to G3, wherein the protein comprises no tag.

G5. The isolated nucleic acid of any one of embodiments G1 to G4.1, wherein the GC content of the nucleotide sequence is about 60% or greater.

G6. The isolated nucleic acid of embodiment G5, wherein the GC3 content of the nucleotide sequence is about 100%.

G7. The isolated nucleic acid of embodiment G5 or G6, comprising the nucleotide sequence of SEQ ID NO: 11.

G8. The isolated nucleic acid of embodiment G7, wherein the nucleotide sequence is 65% or more identical to SEQ ID NO: 10.

G9. The isolated nucleic acid of embodiment G7, wherein the nucleotide sequence is 75% or more identical to SEQ ID NO: 10.

G10. The isolated nucleic acid of any one of embodiments G1 to G9, wherein the viral fusion protein lacks a functional membrane association region.

G11. The isolated nucleic acid of embodiment G10, wherein the viral fusion protein lacks C-terminal transmembrane region amino acids corresponding to amino acids 489 to 539 of SEQ ID NO: 9.

G12. The isolated nucleic acid of any one of embodiments G1 to G11, further comprising a cis-regulatory element in functional association with the nucleotide sequence.

G13. The isolated nucleic acid of embodiment G12, wherein the cis-regulatory element comprises a post transcriptional processing element.

G14. The isolated nucleic acid of embodiment G13, wherein the post transcriptional regulatory element is from woodchuck hepatitis virus.

G15. The isolated nucleic acid of any one of embodiments G1 to G14, which is in an expression vector.

H1. A cell comprising the isolated nucleic acid of embodiment G15.

H2. A cell comprising the nucleotide sequence of any one of embodiments G1 to G11 integrated into cellular DNA.

H3. The cell of any one of embodiments H1 to H2, wherein the viral fusion protein is retained in the cell.

H4. The cell of any one of embodiments H1 to H2, which secretes the viral fusion protein.

H5. The cell of any one of embodiments H1 to H4, which is a mammalian cell.

H6. The cell of embodiment H5, wherein the cell is a non-adherent cell.

H7. The cell of embodiment H5 or H6, wherein the cell is a CHO cell or CHO-derived cell.

H8. The cell of embodiment H7, wherein the cell is a CAT-S cell.

H9. The cell of embodiment H7, wherein the cell is a CHO-S cell.

H10. The cell of embodiment H5, wherein the cell is a Vero cell.

H11. The cell of embodiment H5, wherein the cell is a MRC-5 cell.

H12. The cell of embodiment H5, wherein the cell is a BSR-T7 cell.

H13. The cell of any one of embodiments H1 to H12, wherein the cell synthesizes nucleic acid encoding the viral fusion protein in the nucleus.

H14. The cell of any one of embodiments H1 to H13, which further comprises the cis-regulatory element of any one of embodiments G12 to G14 in functional association with the nucleotide sequence.

I1. A method for expressing a viral fusion protein, comprising contacting a plurality of cells comprising the nucleotide sequence of any one of embodiments G1 to G11 to conditions under which the protein is produced.

I2. The method of embodiment I1, wherein the nucleotide sequence is in an expression vector in the cells.

I3. The method of embodiment I1, wherein the nucleotide sequence is in cellular DNA of the cells.

I4. The method of any one of embodiments I1 to I3, wherein the cells are mammalian cells.

I5. The method of embodiment I4, wherein the cells are non-adherent cells.

I6. The method of embodiment I5, wherein the cells are CHO cells or CHO-derived cells.

I7. The method of embodiment I6, wherein the cells are CAT-S cells.

I8. The method of embodiment I6, wherein the cells are CHO-S cells.

I9. The method of embodiment I4 wherein the cells are Vero cells.

I10. The method of embodiment I4 wherein the cells are MRC-5 cells.

I11. The method of embodiment I4 wherein the cells are BSR-T7 cells.

I12. The method of any one of embodiments I1 to I11, wherein the protein is retained in the cell.

I13. The method of any one of embodiments I1 to I11, wherein the cells secrete the protein.

I14. The method of any one of embodiments I1 to I13, wherein the protein is produced for 7 or more days.

I15. The method of any one of embodiments I1 to I14, wherein the cells are cultured under animal product-free culture conditions.

I16. The method of any one of embodiments I1 to I15, further comprising determining the amount of protein produced by the cells.

I17. The method of any one of embodiments I1 to I16, further comprising isolating the protein.

I18. The method of any one of embodiments I1 to I17, wherein the cell synthesizes nucleic acid encoding the viral fusion protein in the nucleus.

I19. The method of any one of embodiments I1 to I18, wherein the cell further comprises the cis-regulatory element of any one of embodiments G12 to G14 in functional association with the nucleotide sequence of any one of embodiments G1 to G11.

I20. The method of any one of embodiments I1 to I19, which comprises introducing into the cell nucleus the nucleotide sequence of any one of embodiments G1 to G11.

I21. The method of embodiment I20, which comprises introducing into the cell nucleus the cis-regulatory element of any one of embodiments G12 to G14 in functional association with the nucleotide sequence of any one of embodiments G1 to G11.

I22. The method of embodiment I20 or I21, wherein the introducing into the cell nucleus is by nucleotransfection.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, the term “comprising” in each instance encompasses the terms “consisting essentially of” or “consisting of:” The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. Use of the term “about” at the beginning of a string of values modifies each of the values (i.e., “about 1, 2 and 3” refers to about 1, about 2 and about 3). In certain instances units and formatting are expressed in HyperText Markup Language (HTML) format, which can be translated to another conventional format by those skilled in the art (e.g., “.sup.” refers to superscript formatting). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1. An isolated nucleic acid comprising a nucleotide sequence having a GC content of about 51% or greater that encodes a soluble viral fusion protein comprising an amino acid sequence 90% or more identical to SEQ ID NO: 7.

2. The isolated nucleic acid of claim 1, wherein the nucleotide sequence encodes a protein comprising an amino acid sequence 95% or more identical to SEQ ID NO: 7.

3. The isolated nucleic acid of claim 1, wherein the nucleotide sequence encodes a protein comprising an amino acid sequence of SEQ ID NO: 7.

4. The isolated nucleic acid of claim 1, wherein the soluble viral fusion protein lacks a functional membrane association region.

5. The isolated nucleic acid of claim 4, wherein the soluble viral fusion protein lacks C-terminal transmembrane region amino acids corresponding to amino acids 525 to 574 of SEQ ID NO: 2.

6-7. (canceled)

8. The isolated nucleic acid of claim 1, wherein the GC3 content of the nucleotide sequence is 76% or greater.

9. (canceled)

10. The isolated nucleic acid of claim 1, wherein the GC content of the nucleotide sequence is about 58% or greater.

11. The isolated nucleic acid of claim 10, wherein the GC3 content of the nucleotide sequence is about 100%.

12. The isolated nucleic acid of claim 10, comprising the nucleotide sequence of SEQ ID NO: 5.

13-15. (canceled)

16. The isolated nucleic acid of claim 1, further comprising a cis-regulatory element in functional association with the nucleotide sequence.

17. The isolated nucleic acid of claim 16, wherein the cis-regulatory element comprises a post transcriptional processing element.

18. The isolated nucleic acid of claim 17, wherein the post transcriptional regulatory element is from woodchuck hepatitis virus.

19. The isolated nucleic acid of claim 1, which is in an expression vector.

20. A cell comprising the isolated nucleic acid of claim 19.

21-29. (canceled)

30. The cell of claim 20, which is a mammalian cell.

31. The cell of claim 30, wherein the cell is a non-adherent cell.

32. The cell of claim 30, wherein the cell is a CHO cell or CHO-derived cell.

33. The cell of claim 32, wherein the cell is a CAT-S cell.

34-39. (canceled)

40. A method for expressing a soluble viral fusion protein in CHO cells, comprising transfecting the cells with an expression vector that comprises an isolated nucleic acid comprising a nucleotide sequence having a GC content of about 51% or greater that encodes a soluble viral fusion protein comprising an amino acid sequence 90% or more identical to SEQ ID NO: 7.

41-44. (canceled)

46. The method of claim 40, wherein the cells are CAT-S cells.

47-169. (canceled)


SEQUENCE LISTING (52 sequences)

SEQ ID NO: 1 - DNA, 1725 bp, Human respiratory syncytial virus
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagtcagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgccggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 2 - protein, 574 aa, Human respiratory syncytial virus
MELLILKANAITTILTAVTFCFASGQNITEEFYQSTCSAVSKGYLSALRTGWYTSVITIELSNIKKNKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 3 - DNA, 1575 bp, Artificial Sequence
  Description of Artificial Sequence Synthetic polynucleotide
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacaccagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagtcagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgccggtaaatccaccacaaattaa

SEQ ID NO: 4 - DNA, 1575 bp, Artificial Sequence
  Description of Artificial Sequence Synthetic polynucleotide
atggaacttcttattctcaaagccaatgcgattacaacaatccttactgctgtaaccttctgcttcgcatctggacagaatatcaccgaggaattctatcaatccacctgcagcgcggtgtcaaaggggtatctttccgcattgagaacaggttggtatacatccgttattactattgagctgtctaacatcaagaagaataaatgtaatggaactgacgcaaaagtgaagctgatcaagcaggagcttgataagtacaaaaacgctgtgacagaactccagctcctcatgcagagcaccccggcgacgaacaatagagcgcggcgcgagctgcctaggtttatgaattatacccttaacaacgctaagaagacaaacgtgacgctctcaaagaagaggaaacgaaggtttcttggattcctgctcggggtgggatccgctattgcaagcggcgtggcggtttcaaaggtcctccacctggagggggaagtgaacaagattaagtcagcactcctgagtacaaacaaagcagtggtttctctgagcaacggagtgtcagtattgacgagcaaggtgcttgacctcaagaactacattgacaaacagctgctgcccatagtgaacaaacagtcatgctccatctccaatatcgagacagtcatcgaattccagcagaagaacaacagactcctggaaatcacacgggagtttagcgtgaatgcgggcgtaacaactcccgtgtccacctacatgctgacaaattctgagctgctgagtctgataaatgatatgcctattacaaatgaccagaagaagttgatgtccaacaatgtgcaaatagtcagacagcagtcttatagtattatgagcatcatcaaagaggaagttcttgcctatgttgtacaactgcccctctacggggtcatcgacacaccctgttggaagctgcacacctcacctctgtgcaccaccaacacgaaagagggtagcaacatctgtctgactaggactgacaggggttggtactgcgataacgccggtagcgtgtcatttttcccacaagcagagacttgtaaagtacagtccaacagggtcttttgtgacacaatgaattctcttaccctgcccagcgaagttaatctgtgtaacgtcgatatctttaatccaaagtacgattgtaaaatcatgacatctaaaaccgatgtgagcagcagcgttattacaagtcttggcgctatcgtcagctgttacggaaaaaccaagtgcacggcatccaacaagaatagaggcattataaagaccttcagtaatgggtgtgactacgttagcaataagggcgtagacaccgtctccgtaggaaacacactgtactatgtaaataaacaagaaggcaaatccctttatgtgaagggggagcctatcattaatttctacgaccctctggttttcccgagtgacgagttcgatgccagcatatcccaagtgaatgagaaaatcaaccagtccttggcctttataaggaaaagcgatgagcttctgcacaacgtgaatgccggtaaatccaccacaaactag

SEQ ID NO: 5 - DNA, 1575 bp, Artificial Sequence
  Description of Artificial Sequence Synthetic polynucleotide
atggagttgctcatcctcaaggccaacgccatcaccacgatcctcacggccgtcacgttctgcttcgcgtccggccagaacatcaccgaggagttctaccagtcgacgtgcagcgccgtgagcaagggctacctcagcgcgctgaggacgggctggtacaccagcgtcatcacgatcgagttgagcaacatcaagaagaacaagtgcaacggcaccgacgcgaaggtcaagttgatcaagcaggagttggacaagtacaagaacgccgtgaccgagttgcagttgctcatgcagagcacgccggcgacgaacaaccgcgccaggagggagctcccgaggttcatgaactacacgctcaacaacgccaagaagaccaacgtgaccttgagcaagaagaggaagaggaggttcctcggcttcttgttgggcgtcggctcggccatcgccagcggcgtggccgtctcgaaggtcctgcacctggagggcgaggtgaacaagatcaagagcgcgctgctctccacgaacaaggccgtcgtcagcttgtccaacggcgtcagcgtcttgaccagcaaggtgttggacctcaagaactacatcgacaagcagttgttgccgatcgtgaacaagcagagctgcagcatctcgaacatcgagaccgtgatcgagttccagcagaagaacaacaggctgctcgagatcaccagggagttcagcgtcaacgccggcgtcacgacgccggtcagcacctacatgttgaccaacagcgagttgttgtccttgatcaacgacatgccgatcaccaacgaccagaagaagttgatgtccaacaacgtgcagatcgtcaggcagcagagctactcgatcatgtccatcatcaaggaggaggtcttggcctacgtcgtgcagttgccgctgtacggcgtcatcgacacgccctgctggaagctgcacacgtccccgctgtgcacgaccaacacgaaggaggggtccaacatctgcttgaccaggaccgacaggggctggtactgcgacaacgccggctccgtgtcgttcttcccgcaggccgagacctgcaaggtccagtccaaccgcgtcttctgcgacacgatgaacagcttgacgttgccgagcgaggtcaacctctgcaacgtcgacatcttcaaccccaagtacgactgcaagatcatgacgtccaagaccgacgtcagcagctccgtgatcacgtcgctcggcgccatcgtgtcctgctacggcaagaccaagtgcaccgcgtccaacaagaaccgcggcatcatcaagacgttctcgaacgggtgcgactacgtctcgaacaagggggtggacaccgtgtccgtcggcaacacgttgtactacgtcaacaagcaggagggcaagagcctctacgtcaagggcgagccgatcatcaacttctacgacccgttggtcttcccctcggacgagttcgacgcgtcgatctcgcaggtcaacgagaagatcaaccagagcctggcgttcatccggaagtccgacgagttgttgcacaacgtgaacgccggcaagtccaccacgaactaa

SEQ ID NO: 6 - DNA, 3150 bp, Artificial Sequence
  Description of Artificial Sequence Synthetic polynucleotide
atggagttgctcatcctcaaggccaacgccatcaccacgatcctcacggcagtcacattctgtttcgcttctggtcagaacatcactgaggaattctaccaatcgacgtgcagtgcagttagcaagggctatctcagtgctctgagaacgggttggtataccagtgtcatcactatcgagttgagtaacatcaagaagaacaagtgtaacggaaccgatgcgaaggtaaagttgatcaagcaggagttggacaagtacaagaacgctgtaacagagttgcagttgctcatgcagagcacaccagcgacgaacaaccgagccaggagagagctaccaaggttcatgaactacacgctcaacaacgccaagaagaccaacgtgacattgagcaagaagaggaagaggagattcctcggtttcttgttgggtgtcggatctgcaatcgccagtggcgttgctgtctcgaaggtcctgcacctagaaggggaagtgaacaagatcaagagtgctctgctatccacgaacaaggctgtcgtcagcttgtcaaacggagtcagtgtcttgaccagcaaggtgttggacctcaagaactacatcgacaagcagttgttacctatcgtgaacaagcaaagctgcagcatctcaaacatcgagactgtgatcgagttccagcagaagaacaacagactactagagatcaccagggagttcagtgtcaacgcaggtgtaacgacacctgtcagcacttacatgttgactaacagtgagttgttgtcattgatcaacgacatgcctatcaccaacgatcagaagaagttgatgtccaacaacgtgcagatcgtcagacagcagagctactcgatcatgtccatcatcaaggaggaagtcttggcatacgtagtacagttgccactgtatggtgtcatcgacacaccctgctggaagctgcacacgtcccctctatgtacgaccaacacgaaggaagggtccaacatctgcttgaccaggactgacagaggatggtactgcgacaacgcaggatccgtgtcgttcttcccacaggctgagacctgcaaggtccagtccaaccgagtcttctgcgacacgatgaacagcttgacgttgccgagtgaggtaaacctctgcaacgtcgacatcttcaaccccaagtacgactgcaagatcatgacgtccaagaccgatgtcagcagctccgtgatcacatcgctcggagccatcgtgtcatgctacggcaagaccaagtgcacagcgtccaacaagaaccgtggaatcatcaagacgttctcgaacgggtgcgactacgtctcaaacaagggggtggacactgtgtctgtaggcaacacattgtactacgtaaacaagcaggaaggtaagagcctctacgtcaagggtgaaccaatcatcaacttctacgacccgttggtcttcccctctgacgagttcgacgcatcgatctctcaggtcaacgagaagatcaaccagagcctagcattcatccggaagtccgacgagttgttgcacaacgtgaatgccggtaagtccaccacaaactaaatggagttgctcatcctcaaggccaacgccatcaccacgatcctcacggcagtcacattctgtttcgcttctggtcagaacatcactgaggaattctaccaatcgacgtgcagtgcagttagcaagggctatctcagtgctctgagaacgggttggtataccagtgtcatcactatcgagttgagtaacatcaagaagaacaagtgtaacggaaccgatgcgaaggtaaagttgatcaagcaggagttggacaagtacaagaacgctgtaacagagttgcagttgctcatgcagagcacaccagcgacgaacaaccgagccaggagagagctaccaaggttcatgaactacacgctcaacaacgccaagaagaccaacgtgacattgagcaagaagaggaagaggagattcctcggtttcttgttgggtgtcggatctgcaatcgccagtggcgttgctgtctcgaaggtcctgcacctagaaggggaagtgaacaagatcaagagtgctctgctatccacgaacaaggctgtcgtcagcttgtcaaacggagtcagtgtcttgaccagcaaggtgttggacctcaagaactacatcgacaagcagttgttacctatcgtgaacaagcaaagctgcagcatctcaaacatcgagactgtgatcgagttccagcagaagaacaacagactactagagatcaccagggagttcagtgtcaacgcaggtgtaacgacacctgtcagcacttacatgttgactaacagtgagttgttgtcattgatcaacgacatgcctatcaccaacgatcagaagaagttgatgtccaacaacgtgcagatcgtcagacagcagagctactcgatcatgtccatcatcaaggaggaagtcttggcatacgtagtacagttgccactgtatggtgtcatcgacacaccctgctggaagctgcacacgtcccctctatgtacgaccaacacgaaggaagggtccaacatctgcttgaccaggactgacagaggatggtactgcgacaacgcaggatccgtgtcgttcttcccacaggctgagacctgcaaggtccagtccaaccgagtcttctgcgacacgatgaacagcttgacgttgccgagtgaggtaaacctctgcaacgtcgacatcttcaaccccaagtacgactgcaagatcatgacgtccaagaccgatgtcagcagctccgtgatcacatcgctcggagccatcgtgtcatgctacggcaagaccaagtgcacagcgtccaacaagaaccgtggaatcatcaagacgttctcgaacgggtgcgactacgtctcaaacaagggggtggacactgtgtctgtaggcaacacattgtactacgtaaacaagcaggaaggtaagagcctctacgtcaagggtgaaccaatcatcaacttctacgacccgttggtcttcccctctgacgagttcgacgcatcgatctctcaggtcaacgagaagatcaaccagagcctagcattcatccggaagtccgacgagttgttgcacaacgtgaatgccggtaagtccaccacaaactaa

SEQ ID NO: 7 - protein, 524 aa, Artificial Sequence
  Description of Artificial Sequence Synthetic polypeptide
MELLILKANAITTILTAVTFCFASGQNITEEFYQSTCSAVSKGYLSALRTGWYTSVITIELSNIKKNKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 8 - DNA, 1522 bp, Human parainfluenza virus
atgccaacttcaatactgctaattattacaaccatgatcatggcatctttctgccaaatagatatcacaaaactacagcatgtaggtgtattggtcaatagtcccaaaggaatgaagatatcacaaaactttgaaacaagatatctgattttgagcctcataccaaaaatagaagactctaactcttgtggtgaccaacagatcaagcaatacaagaagctattggatagactgatcatccctttatatgatggattaagattacagaaagatgtgatagtaaccaatcaagaatccaatgaaaacactgatcccagaacaaaacgattctttggaggggtaattggaactattgctctgggagtagcaacctcagcacaaattacagcggcagttgctttggttgaagccaagcaggcaagatcagacatcgaaaaactcaaagaagcaattagggacacaaataaagcagtgcagtcagttcagagctccataggaaatctaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatatttacaacatcaacagttgataaatatgatatttatgatctgttatttacagaatcaataaaggtgagagttatagatgttgacttgaatgattactcaatcaccctccaagtcagactccctttattaactaggctgctgaacactcagatctacaaagtagattccatatcatataacattcaaaacagagaatggtatatccctcttcccagccatatcatgacaaaaggggcatttctaggtggagcagatgtcaaagaatgtatagaagcattcagcagctatatatgcccttctgatccaggatttgtattaaaccatgaaatagagagctgcttatcaggaaacatatctcaatgtccaagaaccacagtcacatcagacattgttccaagatatgcatttgtcaatggaggagtggttgcaaactgtataacaaccacttgtacatgcaacggaatcggtaatagaatcaatcaaccacctgatcaaggaataaaaattataacacataaagaatgtagtacaataggtatcaacggaatgctgttcaatacaaataaagaaggaactcttgcattctacacaccaaatgatataacactaaacaattctgttgcacttgatccaattgacatatcaatcgagctcaacaaggccaaatcagatctagaagaatcaaaagaatggataagaaggtcaaatcaaaaactagattccattggaaattggcatcaatctagcactacagtcataattattttgataatgatcattatattgtttataatta

SEQ ID NO: 9 - protein, 539 aa, Human parainfluenza virus
MPTSILLIITTMIMASFCQIDITKLQHVGVLVNSPKGMKISQNFETRYLILSLIPKIEDSNSCGDQQIKQYKKLLDRLIIPLYDGLRLQKDVIVTNQESNENTDPRTKRFFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEIESCLSGNISQCPRTTVTSDIVPRYAFVNGGVVANCITTTCTCNGIGNRINQPPDQGIKIITHKECSTIGINGMLFNTNKEGTLAFYTPNDITLNNSVALDPIDISIELNKAKSDLEESKEWIRRSNQKLDSIGNWHQSSTTVIIILIMIIILFIINVTIITIAIKYYRIQKRNRVDQNDKPYVLTNK

SEQ ID NO: 10 - DNA, 1467 bp, Artificial Sequence
  Description of Artificial Sequence Synthetic polynucleotide
atgccaacttcaatactgctaattattacaaccatgatcatggcatctttctgccaaatagatatcacaaaactacagcatgtaggtgtattggtcaatagtcccaaaggaatgaagatatcacaaaactttgaaacaagatatctgattttgagcctcataccaaaaatagaagactctaactcttgtggtgaccaacagatcaagcaatacaagaagctattggatagactgatcatccctttatatgatggattaagattacagaaagatgtgatagtaaccaatcaagaatccaatgaaaacactgatcccagaacaaaacgattctttggaggggtaattggaactattgctctgggagtagcaacctcagcacaaattacagcggcagttgctttggttgaagccaagcaggcaagatcagacatcgaaaaactcaaagaagcaattagggacacaaataaagcagtgcagtcagttcagagctccataggaaatctaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatatttacaacatcaacagttgataaatatgatatttatgatctgttatttacagaatcaataaaggtgagagttatagatgttgacttgaatgattactcaatcaccctccaagtcagactccctttattaactaggctgctgaacactcagatctacaaagtagattccatatcatataacattcaaaacagagaatggtatatccctcttcccagccatatcatgacaaaaggggcatttctaggtggagcagatgtcaaagaatgtatagaagcattcagcagctatatatgcccttctgatccaggatttgtattaaaccatgaaatagagagctgcttatcaggaaacatatctcaatgtccaagaaccacagtcacatcagacattgttccaagatatgcatttgtcaatggaggagtggttgcaaactgtataacaaccacttgtacatgcaacggaatcggtaatagaatcaatcaaccacctgatcaaggaataaaaattataacacataaagaatgtagtacaataggtatcaacggaatgctgttcaatacaaataaagaaggaactcttgcattctacacaccaaatgatataacactaaacaattctgttgcacttgatccaattgacatatcaatcgagctcaacaaggccaaatcagatctagaagaatcaaaagaatggataagaaggtcaaatcaaaaactagattccattggaaattggcattaa

SEQ ID NO: 11 - DNA, 1467 bp, Artificial Sequence
  Description of Artificial Sequence Synthetic polynucleotide
atgccgacgtccatcctgctgatcatcacgaccatgatcatggcgtcgttctgccagatcgacatcacgaagctccagcacgtcggcgtcttggtcaacagccccaagggcatgaagatctcgcagaacttcgagaccaggtacctgatcttgagcctcatcccgaagatcgaggactcgaactcctgcggcgaccagcagatcaagcagtacaagaagctcttggacaggctgatcatcccgttgtacgacggcttgaggttgcagaaggacgtgatcgtcaccaaccaggagtccaacgagaacaccgaccccaggacgaagcgcttcttcggcggggtcatcggcacgatcgcgctgggggtcgccacctcggcccagatcaccgcggcggtcgcgttggtcgaggccaagcaggcgaggtccgacatcgagaagctcaaggaggccatcagggacacgaacaaggccgtgcagtccgtccagagctccatcggcaacctgatcgtcgcgatcaagtccgtccaggactacgtgaacaaggagatcgtgccgtcgatcgcgaggctcggctgcgaggccgccggcctgcagttgggcatcgcgttgacgcagcactactcggagttgaccaacatcttcggcgacaacatcggctcgttgcaggagaagggcatcaagttgcagggcatcgcgtccttgtaccgcacgaacatcacggagatcttcacgacctcgaccgtcgacaagtacgacatctacgacctgttgttcacggagtcgatcaaggtgagggtcatcgacgtggacttgaacgactactcgatcaccctccaggtcaggctccccttgttgaccaggctgctgaacacgcagatctacaaggtcgactccatctcgtacaacatccagaacagggagtggtacatcccgctgcccagccacatcatgaccaagggggccttcctcggcggcgccgacgtcaaggagtgcatcgaggcgttcagcagctacatctgcccgtcggaccccggcttcgtgttgaaccacgagatcgagagctgcttgtcgggcaacatctcgcagtgcccgaggaccacggtcacgtccgacatcgtgccgaggtacgccttcgtcaacggcggcgtggtcgcgaactgcatcacgaccacgtgcacgtgcaacggcatcggcaacaggatcaaccagccgccggaccagggcatcaagatcatcacgcacaaggagtgcagcaccatcggcatcaacgggatgctgttcaacacgaacaaggagggcacgctggcgttctacacgccgaacgacatcacgctgaacaactcggtcgcgctcgacccgatcgacatctcgatcgagctcaacaaggccaagtcggacctcgaggagtccaaggagtggatcaggaggtcgaaccagaagctcgactccatcggcaactggcactaa

SEQ ID NO: 12 - protein, 488 aa, Artificial Sequence
  Description of Artificial Sequence Synthetic polypeptide
MPTSILLIITTMIMASFCQIDITKLQHVGVLVNSPKGMKISQNFETRYLILSLIPKIEDSNSCGDQQIKQYKKLLDRLIIPLYDGLRLQKDVIVTNQESNENTDPRTKRFFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEIESCLSGNISQCPRTTVTSDIVPRYAFVNGGVVANCITTTCTCNGIGNRINQPPDQGIKIITHKECSTIGINGMLFNTNKEGTLAFYTPNDITLNNSVALDPIDISIELNKAKSDLEESKEWIRRSNQKLDSIGNWH

SEQ ID NO: 13 - DNA, 1485 bp, Artificial Sequence
  Description of Artificial Sequence Synthetic polynucleotide
atgccaacttcaatactgctaattattacaaccatgatcatggcatctttctgccaaatagatatcacaaaactacagcatgtaggtgtattggtcaatagtcccaaaggaatgaagatatcacaaaactttgaaacaagatatctgattttgagcctcataccaaaaatagaagactctaactcttgtggtgaccaacagatcaagcaatacaagaagctattggatagactgatcatccctttatatgatggattaagattacagaaagatgtgatagtaaccaatcaagaatccaatgaaaacactgatcccagaacaaaacgattctttggaggggtaattggaactattgctctgggagtagcaacctcagcacaaattacagcggcagttgctttggttgaagccaagcaggcaagatcagacatcgaaaaactcaaagaagcaattagggacacaaataaagcagtgcagtcagttcagagctccataggaaatctaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatatttacaacatcaacagttgataaatatgatatttatgatctgttatttacagaatcaataaaggtgagagttatagatgttgacttgaatgattactcaatcaccctccaagtcagactccctttattaactaggctgctgaacactcagatctacaaagtagattccatatcatataacattcaaaacagagaatggtatatccctcttcccagccatatcatgacaaaaggggcatttctaggtggagcagatgtcaaagaatgtatagaagcattcagcagctatatatgcccttctgatccaggatttgtattaaaccatgaaatagagagctgcttatcaggaaacatatctcaatgtccaagaaccacagtcacatcagacattgttccaagatatgcatttgtcaatggaggagtggttgcaaactgtataacaaccacttgtacatgcaacggaatcggtaatagaatcaatcaaccacctgatcaaggaataaaaattataacacataaagaatgtagtacaataggtatcaacggaatgctgttcaatacaaataaagaaggaactcttgcattctacacaccaaatgatataacactaaacaattctgttgcacttgatccaattgacatatcaatcgagctcaacaaggccaaatcagatctagaagaatcaaaagaatggataagaaggtcaaatcaaaaactagattccattggaaattggcatcaccaccatcaccatcactaa

SEQ ID NO: 14 - DNA, 1485 bp, Artificial Sequence
  Description of Artificial Sequence Synthetic polynucleotide
atgccgacgtccatcctgctgatcatcacgaccatgatcatggcgtcgttctgccagatcgacatcacgaagctccagcacgtcggcgtcttggtcaacagccccaagggcatgaagatctcgcagaacttcgagaccaggtacctgatcttgagcctcatcccgaagatcgaggactcgaactcctgcggcgaccagcagatcaagcagtacaagaagctcttggacaggctgatcatcccgttgtacgacggcttgaggttgcagaaggacgtgatcgtcaccaaccaggagtccaacgagaacaccgaccccaggacgaagcgcttcttcggcggggtcatcggcacgatcgcgctgggggtcgccacctcggcccagatcaccgcggcggtcgcgttggtcgaggccaagcaggcgaggtccgacatcgagaagctcaaggaggccatcagggacacgaacaaggccgtgcagtccgtccagagctccatcggcaacctgatcgtcgcgatcaagtccgtccaggactacgtgaacaaggagatcgtgccgtcgatcgcgaggctcggctgcgaggccgccggcctgcagttgggcatcgcgttgacgcagcactactcggagttgaccaacatcttcggcgacaacatcggctcgttgcaggagaagggcatcaagttgcagggcatcgcgtccttgtaccgcacgaacatcacggagatcttcacgacctcgaccgtcgacaagtacgacatctacgacctgttgttcacggagtcgatcaaggtgagggtcatcgacgtggacttgaacgactactcgatcaccctccaggtcaggctccccttgttgaccaggctgctgaacacgcagatctacaaggtcgactccatctcgtacaacatccagaacagggagtggtacatcccgctgcccagccacatcatgaccaagggggccttcctcggcggcgccgacgtcaaggagtgcatcgaggcgttcagcagctacatctgcccgtcggaccccggcttcgtgttgaaccacgagatcgagagctgcttgtcgggcaacatctcgcagtgcccgaggaccacggtcacgtccgacatcgtgccgaggtacgccttcgtcaacggcggcgtggtcgcgaactgcatcacgaccacgtgcacgtgcaacggcatcggcaacaggatcaaccagccgccggaccagggcatcaagatcatcacgcacaaggagtgcagcaccatcggcatcaacgggatgctgttcaacacgaacaaggagggcacgctggcgttctacacgccgaacgacatcacgctgaacaactcggtcgcgctcgacccgatcgacatctcgatcgagctcaacaaggccaagtcggacctcgaggagtccaaggagtggatcaggaggtcgaaccagaagctcgactccatcggcaactggcaccaccaccatcaccatcactaa

SEQ ID NO: 15 - protein, 494 aa, Artificial Sequence
  Description of Artificial Sequence Synthetic polypeptide
MPTSILLIITTMIMASFCQIDITKLQHVGVLVNSPKGMKISQNFETRYLILSLIPKIEDSNSCGDQQIKQYKKLLDRLIIPLYDGLRLQKDVIVTNQESNENTDPRTKRFFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEIESCLSGNISQCPRTTVTSDIVPRYAFVNGGVVANCITTTCTCNGIGNRINQPPDQGIKIITHKECSTIGINGMLFNTNKEGTLAFYTPNDITLNNSVALDPIDISIELNKAKSDLEESKEWIRRSNQKLDSIGNWHHHHHHH

SEQ ID NO: 16 - DNA, 1725 bp, Artificial Sequence
  Description of Artificial Sequence Synthetic polynucleotide
atggaacttcttattctcaaagccaatgcgattacaacaatccttactgctgtaaccttctgcttcgcatctggacagaatatcaccgaggaattctatcaatccacctgcagcgcggtgtcaaaggggtatctttccgcattgagaacaggttggtatacatccgttattactattgagctgtctaacatcaagaagaataaatgtaatggaactgacgcaaaagtgaagctgatcaagcaggagcttgataagtacaaaaacgctgtgacagaactccagctcctcatgcagagcaccccggcgacgaacaatagagcgcggcgcgagctgcctaggtttatgaattatacccttaacaacgctaagaagacaaacgtgacgctctcaaagaagaggaaacgaaggtttcttggattcctgctcggggtgggatccgctattgcaagcggcgtggcggtttcaaaggtcctccacctggagggggaagtgaacaagattaagtcagcactcctgagtacaaacaaagcagtggtttctctgagcaacggagtgtcagtattgacgagcaaggtgcttgacctcaagaactacattgacaaacagctgctgcccatagtgaacaaacagtcatgctccatctccaatatcgagacagtcatcgaattccagcagaagaacaacagactcctggaaatcacacgggagtttagcgtgaatgcgggcgtaacaactcccgtgtccacctacatgctgacaaattctgagctgctgagtctgataaatgatatgcctattacaaatgaccagaagaagttgatgtccaacaatgtgcaaatagtcagacagcagtcttatagtattatgagcatcatcaaagaggaagttcttgcctatgttgtacaactgcccctctacggggtcatcgacacaccctgttggaagctgcacacctcacctctgtgcaccaccaacacgaaagagggtagcaacatctgtctgactaggactgacaggggttggtactgcgataacgccggtagcgtgtcatttttcccacaagcagagacttgtaaagtacagtccaacagggtcttttgtgacacaatgaattctcttaccctgcccagcgaagttaatctgtgtaacgtcgatatctttaatccaaagtacgattgtaaaatcatgacatctaaaaccgatgtgagcagcagcgttattacaagtcttggcgctatcgtcagctgttacggaaaaaccaagtgcacggcatccaacaagaatagaggcattataaagaccttcagtaatgggtgtgactacgttagcaataagggcgtagacaccgtctccgtaggaaacacactgtactatgtaaataaacaagaaggcaaatccctttatgtgaagggggagcctatcattaatttctacgaccctctggttttcccgagtgacgagttcgatgccagcatatcccaagtgaatgagaaaatcaaccagtccttggcctttataaggaaaagcgatgagcttctgcacaacgtgaatgccggtaaatccaccacaaacataatgatcaccactatcattatcgtcattattgtgatcttgctgagcctcatcgctgtggggctcctcttgtattgcaaagcccgctcaaccccagtcactctctctaaagaccaactgtctgggatcaataacatagccttttcaaattag

SEQ ID NO: 17 - DNA, 1725 bp, Artificial Sequence
  Description of Artificial Sequence Synthetic polynucleotide
atggagttgctcatcctcaaggccaacgccatcaccacgatcctcacggccgtcacgttctgcttcgcgtccggccagaacatcaccgaggagttctaccagtcgacgtgcagcgccgtgagcaagggctacctcagcgcgctgaggacgggctggtacaccagcgtcatcacgatcgagttgagcaacatcaagaagaacaagtgcaacggcaccgacgcgaaggtcaagttgatcaagcaggagttggacaagtacaagaacgccgtgaccgagttgcagttgctcatgcagagcacgccggcgacgaacaaccgcgccaggagggagctcccgaggttcatgaactacacgctcaacaacgccaagaagaccaacgtgaccttgagcaagaagaggaagaggaggttcctcggcttcttgttgggcgtcggctcggccatcgccagcggcgtggccgtctcgaaggtcctgcacctggagggcgaggtgaacaagatcaagagcgcgctgctctccacgaacaaggccgtcgtcagcttgtccaacggcgtcagcgtcttgaccagcaaggtgttggacctcaagaactacatcgacaagcagttgttgccgatcgtgaacaagcagagctgcagcatctcgaacatcgagaccgtgatcgagttccagcagaagaacaacaggctgctcgagatcaccagggagttcagcgtcaacgccggcgtcacgacgccggtcagcacctacatgttgaccaacagcgagttgttgtccttgatcaacgacatgccgatcaccaacgaccagaagaagttgatgtccaacaacgtgcagatcgtcaggcagcagagctactcgatcatgtccatcatcaaggaggaggtcttggcctacgtcgtgcagttgccgctgtacggcgtcatcgacacgccctgctggaagctgcacacgtccccgctgtgcacgaccaacacgaaggaggggtccaacatctgcttgaccaggaccgacaggggctggtactgcgacaacgccggctccgtgtcgttcttcccgcaggccgagacctgcaaggtccagtccaaccgcgtcttctgcgacacgatgaacagcttgacgttgccgagcgaggtcaacctctgcaacgtcgacatcttcaaccccaagtacgactgcaagatcatgacgtccaagaccgacgtcagcagctccgtgatcacgtcgctcggcgccatcgtgtcctgctacggcaagaccaagtgcaccgcgtccaacaagaaccgcggcatcatcaagacgttctcgaacgggtgcgactacgtctcgaacaagggggtggacaccgtgtccgtcggcaacacgttgtactacgtcaacaagcaggagggcaagagcctctacgtcaagggcgagccgatcatcaacttctacgacccgttggtcttcccctcggacgagttcgacgcgtcgatctcgcaggtcaacgagaagatcaaccagagcctggcgttcatccggaagtccgacgagttgttgcacaacgtgaacgccggcaagtccaccacgaacatcatgatcacgacgatcatcatcgtgatcatcgtgatcttgttgtcgttgatcgccgtcggcctgctcttgtactgcaaggccaggagcacgcccgtcacgctgagcaaggaccagctgagcggcatcaacaacatcgcgttcagcaactaa

SEQ ID NO: 18 - DNA, 33 bp, Artificial Sequence
  Description of Artificial Sequence Synthetic primer
gcatgagctcatggagttgctaatcctcaaagc

SEQ ID NO: 19 - DNA, 39 bp, Artificial Sequence
  Description of Artificial Sequence Synthetic primer
gcatctcgaggttactaaatgcaatattatttataccac

SEQ ID NO: 20 - DNA, 40 bp, Artificial Sequence
  Description of Artificial Sequence Synthetic primer
gcatgagctcatggaacttcttattctcaaagccaatgcg

SEQ ID NO: 21 - DNA, 44 bp, Artificial Sequence
  Description of Artificial Sequence Synthetic primer
gcatctcgagctaatttgaaaaggctatgttattgatcccagac

SEQ ID NO: 22 - DNA, 33 bp, Artificial Sequence
  Description of Artificial Sequence Synthetic primer
gcatgctagcatggagttgctcatcctcaaggc

SEQ ID NO: 23 - DNA, 35 bp, Artificial Sequence
  Description of Artificial Sequence Synthetic primer
gcataagcttttagttgctgaacgcgatgttgttg

SEQ ID NO: 24 - DNA, 31 bp, Artificial Sequence
  Description of Artificial Sequence Synthetic primer
ggccggccatggagttgctaatcctcaaagc

SEQ ID NO: 25 - DNA, 32 bp, Artificial Sequence
  Description of Artificial Sequence Synthetic primer
cctgcaggttaatttgtggtggatttaccggc

SEQ ID NO: 26 - DNA, 32 bp, Artificial Sequence
  Description of Artificial Sequence Synthetic primer
ggccggccatggaacttcttattctcaaagcc

SEQ ID NO: 27 - DNA, 32 bp, Artificial Sequence
  Description of Artificial Sequence Synthetic primer
cctgcaggttagtttgtggtggatttaccggc

SEQ ID NO: 28 - DNA, 32 bp, Artificial Sequence
  Description of Artificial Sequence Synthetic primer
ggccggccatggagttgctcatcctcaaggcc

SEQ ID NO: 29 - DNA, 33 bp, Artificial Sequence
  Description of Artificial Sequence Synthetic primer
cctgcaggttagttcgtggtggacttgccggcg

SEQ ID NO: 30 - DNA, 44 bp, Artificial Sequence
  Description of Artificial Sequence Synthetic primer
gcatggccggccatgccaacttcaatactgctaattattacaac

SEQ ID NO: 31 - DNA, 39 bp, Artificial Sequence
  Description of Artificial Sequence Synthetic primer
gcatcctgcaggttaatgccaatttccaatggaatctag

SEQ ID NO: 32 - DNA, 30 bp, Artificial Sequence
  Description of Artificial Sequence Synthetic primer
gcaggccggccatgccgacgtccatcctgc

SEQ ID NO: 33 - DNA, 33 bp, Artificial Sequence
  Description of Artificial Sequence Synthetic primer
gcacctgcaggttagtgccagttgccgatggag

SEQ ID NO: 34 - DNA, 44 bp, Artificial Sequence; identical to SEQ ID NO: 30
  Description of Artificial Sequence Synthetic primer

SEQ ID NO: 35 - DNA, 57 bp, Artificial Sequence
  Description of Artificial Sequence Synthetic primer
gcatcctgcaggttagtgatggtgatggtggtgatgccaatttccaatggaatctag

SEQ ID NO: 36 - DNA, 30 bp, Artificial Sequence; identical to SEQ ID NO: 32
  Description of Artificial Sequence Synthetic primer

SEQ ID NO: 37 - DNA, 52 bp, Artificial Sequence
  Description of Artificial Sequence Synthetic primer
gcatcctgcaggttagtgatggtgatggtggtggtgccagttgccgatggag

SEQ ID NO: 38 - protein, 9 aa, Artificial Sequence
  Description of Artificial Sequence Synthetic peptide
DYKDDDDKG

SEQ ID NO: 39 - protein, 14 aa, Artificial Sequence
  Description of Artificial Sequence Synthetic peptide
GKPIPNPLLGLDST

SEQ ID NO: 40 - protein, 10 aa, Artificial Sequence
  Description of Artificial Sequence Synthetic peptide
EQKLISEEDL

SEQ ID NO: 41 - protein, 11 aa, Herpes simplex virus
QPELAPEDPED

SEQ ID NO: 42 - protein, 9 aa, Influenza A virus
YPYDVPDYA

SEQ ID NO: 43 - protein, 11 aa, Vesicular stomatitis virus
YTDIEMNRLGK

SEQ ID NO: 44 - protein, 6 aa, Artificial Sequence
  Description of Artificial Sequence Synthetic 6xHis tag
HHHHHH

SEQ ID NO: 45 - protein, 7 aa, Artificial Sequence
  Description of Artificial Sequence Synthetic peptide
CCXXXCC

SEQ ID NO: 46 - protein, 6 aa, Artificial Sequence
  Description of Artificial Sequence Synthetic peptide
CCPGCC

SEQ ID NO: 47 - protein, 4 aa, Unknown
  Description of Unknown Factor Xa recognition site peptide
IXGR

SEQ ID NO: 48 - protein, 6 aa, Unknown
  Description of Unknown Thrombin recognition site peptide
LVPRGS

SEQ ID NO: 49 - protein, 5 aa, Unknown
  Description of Unknown Enterokinase recognition site peptide
DDDDK

SEQ ID NO: 50 - protein, 7 aa, Unknown
  Description of Unknown TEV protease recognition site peptide
ENLYFQG

SEQ ID NO: 51 - protein, 8 aa, Unknown
  Description of Unknown PreScission protease recognition site peptide
LEVLFQGP

SEQ ID NO: 52 - protein, 8 aa, Artificial Sequence
  Description of Artificial Sequence Synthetic His tag
HHHHHHHH